(19) United States
(12) Patent Application Publication
(10) Pub. No.: US 2026/0112453 A1
(43) Pub. Date: Apr. 23, 2026

(54) SYSTEMS AND METHODS FOR CONTEXT-FREE CELL TYPE DECONVOLUTION OF MULTI-SCALE TRANSCRIPTOMIC DATA

(71) Applicant: Icahn School of Medicine at Mount Sinai, New York, NY (US)

(72) Inventors: Daniel M. Charytonowicz, New York, NY (US); Robert S. Sebra, New York, NY (US)

(21) Appl. No.: 19/115,155

(22) PCT Filed: Sep. 27, 2023
(86) PCT No.: PCT/US2023/075251

§ 371 Date: Mar. 25, 2025

Related U.S. Application Data
(60) Provisional application No. 63/410,537, filed on Sep. 27, 2022.

(51) Int. Cl. G16B 40/20; G06N 3/09; G16B 25/10

(52) U.S. Cl. CPC G16B 40/20; G16B 25/10; G06N 3/09

(57) ABSTRACT

Systems and methods for training a context-free model to determine cell type fractions are provided. A training set is obtained comprising, for each of a plurality of data stores, for respective each cell represented in the data store, a dataset comprising abundance values for cellular constituents associated with the respective cell. Pseudobulk training mixtures are formed from the training set. For each mixture, the abundance value for each cellular constituent is averaged across abundance datasets of the cells represented by the respective mixture thereby forming an averaged abundance dataset for the mixture. For each mixture, a corresponding averaged abundance dataset is inputted into the model thereby obtaining a respective plurality of calculated cell type fractions, each fraction for a different cell type. Model parameters are adjusted based on differences between calculated cell type fractions and mixture fraction ratios for each unique cell type in the respective mixture.

### CROSS REFERENCE TO RELATED APPLICATIONS

This application claims priority to U.S. Provisional Patent Application Ser. No. 63/410,537, entitled “Systems and Methods for Context-Free Cell Type Deconvolution of Multi-Scale Transcriptomic Data,” filed Sep. 27, 2022, which is hereby incorporated by reference in its entirety for all purposes.

### TECHNICAL FIELD

The disclosure relates generally to determining cell type proportions for a plurality of cell types for a sample with a context-free model.

### BACKGROUND

The ability to measure expression of the coding genome has revolutionized the study of human disease [1]. Recently, the appreciation of inter-patient cellular heterogeneity has led to methods such as single-cell RNA Sequencing (scRNA-Seq) being introduced to increase study resolution [2]. There is now interest in measuring the influence of spatial cellular organization on pathophysiology, which is being accomplished through spatial transcriptomics (ST). Broadly, ST platforms can be divided into two categories. Targeted, high-resolution approaches such as MERFISH [3], split-FISH [4], or OligoFISSEQ [5] can profile tens to hundreds of genes using variations of nucleic-acid hybridization techniques at the subcellular level. Alternatively, whole-transcriptome, lower-resolution approaches such as Slide-Seq [6], Visium [7], DBiT-seq [8], or Stereo-seq [9] function via spatial-aware RNA capture and sequencing. The unbiased nature of whole-transcriptome approaches makes them appealing for early-stage discovery and hypothesis-generation.

Resolution of whole-transcriptome spatial platforms varies, ranging from 10 um for Slide-Seq to 55 um for Visium. While the density of capture arrays is increasing, spatial capture spots nevertheless contain RNA content eluted from several single cells. Differences in gene expression are driven in-part by varying cell type mixtures and levels of individual cell transcript expression. As such, deconvolving of cell type fractions, for each spot, would improve interpretability and analysis of differential gene expression patterns. Multiple machine learning methods addressing cellular deconvolution have been introduced. Earlier approaches focusing on bulk-RNA-Seq include methods such as DSA [10], MuSiC [11], CIBERSORT/CIBERSORTx [12,13], Scaden [14], DeconRNASeq [15], and SCDC [16]. The emergence of spatial transcriptomics has ushered in a new generation of deconvolution algorithms, notably Cell2Location [17], SPOTLight [18], Stereoscope [19], SpatialDWLS [20], DSTG [21], STDeconvolve [22], and RCTD [23].

A significant limitation of such approaches is the requirement for a reference profile of cell type expression. Meta-analyses of RNA-seq deconvolution algorithms have shown that choice of reference is more important than methodology in determining deconvolution performance [24]. The choice of cell types to include in a reference is not always apparent, and collecting matched samples for reference generation is not always possible. Furthermore, the use of general scRNA-Seq “atlases” as references may not be appropriate when transcriptional differences due to experimental or disease-related factors confound cell type expression patterns.

Given the above background, what is needed in the art are improved methods for deconvolving cell types in samples.

### SUMMARY

The present disclosure provides improved methods for deconvolving cell types in samples.

One aspect of the present disclosure provides a method of training a context-free model to determine a plurality of cell type fractions in a sample. The method comprises aggregating, for each respective data store in plurality of data stores, for each respective cell in a respective population of cells represented in the respective data store, a respective abundance dataset comprising a respective abundance value for each respective cellular constituent in a corresponding plurality of cellular constituents associated with the respective cell, into a training set. Each data store in the plurality of data stores contributes an abundance dataset for each of a corresponding plurality of cells to the training set. In some embodiments, each corresponding plurality of cellular constituents in each respective cell is at least 50 cellular constituents. In some embodiments, the training set includes abundance data for twenty or more cell types. In some embodiments, the training set includes abundance data for cells from ten or more tissue types;

A plurality of pseudobulk training mixtures are formed from the training set. Each respective pseudobulk training mixture is formed by a first procedure comprising (i) determining a number T on a random basis between a first lower threshold and a first upper threshold, (ii) determining a number of unique cell types N between a second lower threshold and a second upper threshold, and (iii) determining a corresponding mixture fraction ratio F, on a random basis for each respective unique cell type i in the number of unique cells types N. Further in the first procedure, for each respective unique cell type i in the number of unique cell types N, the abundance dataset of up to Fix T cells of the respective unique cell is selected on a random basis from the training set thereby obtaining a respective plurality of cells represented by the respective pseudobulk training mixture. The respective abundance value for each respective corresponding cellular constituent is averaged across each abundance dataset of the respective plurality of cells represented by the respective pseudobulk training mixture thereby forming an averaged abundance dataset for the respective pseudobulk training mixture comprising averaged abundance for a set of cellular constituents.

The context-free model is trained by performing, for each respective pseudobulk training mixture in the plurality of pseudobulk training mixtures, a second procedure. The second procedure comprises inputting the averaged abundance dataset for the respective pseudobulk training mixture into the context-free model thereby obtaining a respective plurality of calculated cell type fractions, each calculated cell type fraction in the respective plurality of calculated cell type fractions for a different cell type in a plurality of cell types, and adjusting a value of one or more parameters in the plurality of parameters based on a difference between (i) the respective plurality of calculated cell type fractions and (ii) the mixture fraction ratio Fᵢ for each respective unique cell type i in the number of unique cells types represented by the respective pseudobulk training mixture.

In some embodiments, once the model is trained, the method further comprises obtaining a test sample, in electronic form, where the test sample comprises a respective abundance value for each respective cellular constituent in a corresponding plurality of cellular constituents obtained from a bulk data assay. The respective abundance value for each respective cellular constituent in the corresponding plurality of cellular constituents is inputted into the context-free model thereby obtaining a plurality of test calculated cell type fractions. Each test calculated cell type fraction in the respective plurality of test calculated cell type fractions for a different cell type in the plurality of cell types.

In some embodiments, once the model is trained, the method further comprises obtaining a test sample, in electronic form, where the test sample comprises a respective abundance value for each respective cellular constituent in a corresponding plurality of cellular constituents for each respective test cell in a plurality of test cells obtained from a single cell or a single-nuclei assay. For each respective test cell in the plurality of test cells, the respective abundance value for each respective cellular constituent in the corresponding plurality of cellular constituents of the respective test cell is inputted into the context-free model thereby obtaining a respective plurality of test calculated cell type probabilities, each test calculated cell type probability in the respective plurality of test calculated cell type probabilities for a different cell type in the plurality of cell types. In some such embodiments, each respective plurality of test calculated cell type probabilities is averaged across the plurality of test cells to form a plurality of test calculated cell type fractions representative of the test sample, each respective test calculated cell type fraction in the plurality of test calculated cell type fractions corresponding to a different cell type in the plurality of cell types.

In some embodiments, the plurality of cell types is between 50 cell types and 2000 cell types, or between 200 cell types and 1500 cell types, or between 500 cell types and 1000 cell types, greater than 100 cell types, greater than 200 cell types, or greater than 1000 cell types.

In some embodiments, the respective plurality of cells represented by the respective pseudobulk training mixture includes cells from two or more data stores, three or more data stores, or five or more data stores in the plurality of data stores.

In some embodiments, the adjusting a value of one or more parameters in the plurality of parameters based on the difference is performed by backpropagation through all or a subset of the plurality of parameters of the context-free model.

In some embodiments, the plurality of pseudobulk training mixtures comprises 100,000 pseudobulk training mixtures, 500,000 pseudobulk training mixtures, 1×10⁶ pseudobulk training mixtures, 5×10⁶ pseudobulk training mixtures, 1×10⁷ pseudobulk training mixtures, or 5×10⁷ pseudobulk training mixtures.

In some embodiments, the training described above is repeated. In some embodiments, the training described above is repeated a plurality of times. In some embodiments, the training described above is repeated one or more times, two or more times, three or more times, four or more times, 10 or more times, between 15 and 100 times, or between 40 and 1000 times.

In some embodiments, the context-free model is a multiple layer fully connected neural network. In some such embodiments, a final layer of the context-free model comprises a neuron for each cell type in the plurality of cell types with a softmax activation function yielding the respective plurality of calculated cell type fractions summing to one.

In some embodiments, the plurality of data stores comprises 50 or more data stores, 100 or more data stores, or 1000 or more data stores.

In some embodiments, the respective abundance dataset comprising a respective abundance value for each respective cellular constituent in the corresponding plurality of cellular constituents associated with the respective cell is single cell or single-nuclei RNA-seq data for a plurality of genes.

In some embodiments, the respective abundance dataset comprising a respective abundance value for each respective cellular constituent in the corresponding plurality of cellular constituents associated with the respective cell is chromatin data.

In some embodiments, the respective abundance dataset comprising a respective abundance value for each respective cellular constituent in the corresponding plurality of cellular constituents associated with the respective cell is protein expression data.

In some embodiments, the context-free model has a plurality of trainable parameters. In some embodiments the plurality of trainable parameters comprises 10,000 trainable parameters, 100,000 trainable parameter, 1×10⁶ trainable parameters, 1×10⁷ trainable parameters, or 1×10⁸ trainable parameters.

In some embodiments, the inputting the averaged abundance dataset for the respective pseudobulk training mixture into the context-free model, during the training, sets a first percentage of the set of cellular constituents to zero on a random basis (e.g., between 10 percent and 30 percent).

In some embodiments, the set of cellular constituents consists of between 400 cellular constituents and 50,000 cellular constituents.

In some embodiments, the context-free model comprises a plurality of fully connected layers, each respective fully connected layer in the plurality of fully connected layers comprising a corresponding plurality of neurons, and each respective neuron in the corresponding plurality of neurons analyzed by a corresponding activation function. In some such embodiments, the corresponding activation function is a Tanh function, a rectified linear unit (RELU), or exponential linear unit (ELU). In some such embodiments, the plurality of fully connected layers is between three and twenty fully connected layers. In some such embodiments, there is at least one dropout layer between a first fully connected layer and a second fully connected layer in the plurality of fully connected layers that sets a second percentage (e.g., between 5 percent and 15 percent) of the neuron values of the first fully connected layer to zero on a random basis.

Another aspect of the present disclosure provides a computing system comprising one or more processors and a memory. The memory stores one or more programs for execution by the one or more processors. The one or more programs singularly or collectively comprise instructions for executing a method of training a context-free model to determine a plurality of cell type fractions in a sample. The method comprises aggregating, for each respective data store in plurality of data stores, for each respective cell in a respective population of cells represented in the respective data store, a respective abundance dataset comprising a respective abundance value for each respective cellular constituent in a corresponding plurality of cellular constituents associated with the respective cell, into a training set. Each data store in the plurality of data stores contributes an abundance dataset for each of a corresponding plurality of cells to the training set. A plurality of pseudobulk training mixtures is formed from the training set, where each respective pseudobulk training mixture is formed by a first procedure comprising (i) determining a number T on a random basis between a first lower threshold and a first upper threshold, (ii) determining a number of unique cell types N between a second lower threshold and a second upper threshold, and (iii) determining a corresponding mixture fraction ratio F, on a random basis for each respective unique cell type i in the number of unique cells types N. The first procedure further comprises, for each respective unique cell type i in the number of unique cell types N, selecting the abundance dataset of up to Fᵢ+T cells of the respective unique cell on a random basis from the training set thereby obtaining a respective plurality of cells represented by the respective pseudobulk training mixture, and averaging the respective abundance value for each respective corresponding cellular constituent across each abundance dataset of the respective plurality of cells represented by the respective pseudobulk training mixture thereby forming an averaged abundance dataset for the respective pseudobulk training mixture comprising averaged abundance for a set of cellular constituents. A context-free model is trained by performing, for each respective pseudobulk training mixture in the plurality of pseudobulk training mixtures, a second procedure comprising: (a) inputting the averaged abundance dataset for the respective pseudobulk training mixture into the context-free model thereby obtaining a respective plurality of calculated cell type fractions, each calculated cell type fraction in the respective plurality of calculated cell type fractions for a different cell type in a plurality of cell types, and (b) adjusting a value of one or more parameters in the plurality of parameters based on a difference between (i) the respective plurality of calculated cell type fractions and (ii) the mixture fraction ratio Fᵢ for each respective unique cell type i in the number of unique cells types represented by the respective pseudobulk training mixture.

Another aspect of the present disclosure provides a non-transitory computer readable storage medium stored on a computing device. The computing device comprises one or more processors and a memory. The memory stores one or more programs for execution by the one or more processors. The one or more programs singularly or collectively comprise instructions for executing a method of training a context-free model to determine a plurality of cell type fractions in a sample. The method comprises aggregating, for each respective data store in plurality of data stores, for each respective cell in a respective population of cells represented in the respective data store, a respective abundance dataset comprising a respective abundance value for each respective cellular constituent in a corresponding plurality of cellular constituents associated with the respective cell, into a training set. Each data store in the plurality of data stores contributes an abundance dataset for each of a corresponding plurality of cells to the training set. A plurality of pseudobulk training mixtures is formed from the training set, where each respective pseudobulk training mixture is formed by a first procedure comprising (i) determining a number T on a random basis between a first lower threshold and a first upper threshold, (ii) determining a number of unique cell types N between a second lower threshold and a second upper threshold, and (iii) determining a corresponding mixture fraction ratio F, on a random basis for each respective unique cell type i in the number of unique cells types N. The first procedure further comprises, for each respective unique cell type i in the number of unique cell types N, selecting the abundance dataset of up to Fᵢ+T cells of the respective unique cell on a random basis from the training set thereby obtaining a respective plurality of cells represented by the respective pseudobulk training mixture, and averaging the respective abundance value for each respective corresponding cellular constituent across each abundance dataset of the respective plurality of cells represented by the respective pseudobulk training mixture thereby forming an averaged abundance dataset for the respective pseudobulk training mixture comprising averaged abundance for a set of cellular constituents. A context-free model is trained by performing, for each respective pseudobulk training mixture in the plurality of pseudobulk training mixtures, a second procedure comprising: (a) inputting the averaged abundance dataset for the respective pseudobulk training mixture into the context-free model thereby obtaining a respective plurality of calculated cell type fractions, each calculated cell type fraction in the respective plurality of calculated cell type fractions for a different cell type in a plurality of cell types, and (b) adjusting a value of one or more parameters in the plurality of parameters based on a difference between (i) the respective plurality of calculated cell type fractions and (ii) the mixture fraction ratio Fᵢ for each respective unique cell type i in the number of unique cells types represented by the respective pseudobulk training mixture.

Another aspect of the present disclosure provides a method of determining a plurality of cell type fractions for a plurality of cell types for a sample. The method comprises obtaining a test sample, in electronic form, where the test sample comprises a respective abundance value for each respective cellular constituent in a plurality of cellular constituents obtained from a bulk data assay. The method further comprises inputting the respective abundance value for each respective cellular constituent in the corresponding plurality of cellular constituents into a context-free model thereby obtaining a plurality of calculated cell type fractions, each calculated cell type fraction in the respective plurality of calculated cell type fractions for a different cell type in the plurality of cell types. In some such embodiments, the plurality of cell types comprises 300 or more different cell types and the plurality of cellular constituents comprises 400 or more cellular constituents. In some embodiments, the context-free model comprises a plurality of fully connected layers, each respective fully connected layer in the plurality of fully connected layers comprising a corresponding plurality of neurons, and each respective neuron in the corresponding plurality of neurons analyzed by a corresponding activation function, a final layer of the context-free model comprises a neuron for each cell type in the plurality of cell types with a softmax activation function yielding the respective plurality of calculated cell type fractions summing to one, and the context-free model comprises 10,000 trainable parameters.

Another aspect of the present disclosure provides a method of determining cell type proportions for a plurality of cell types for a sample. The method comprises obtaining a test sample, in electronic form, where the test sample comprises a respective abundance value for each respective cellular constituent in a corresponding plurality of cellular constituents for each respective test cell in a plurality of test cells obtained from a single cell assay, and, for each respective test cell in the plurality of test cells, inputting the respective abundance value for each respective cellular constituent in the corresponding plurality of cellular constituents into the context-free model thereby obtaining a respective plurality of test calculated cell type probabilities, each test calculated cell type probability in the respective plurality of test calculated cell type probabilities for a different cell type in the plurality of cell types. In some such embodiments, the plurality of cell types comprises 300 or more different cell types and the plurality of cellular constituents comprises 400 or more cellular constituents. In some embodiments, the context-free model comprises a plurality of fully connected layers, each respective fully connected layer in the plurality of fully connected layers comprising a corresponding plurality of neurons, and each respective neuron in the corresponding plurality of neurons analyzed by a corresponding activation function, a final layer of the context-free model comprises a neuron for each cell type in the plurality of cell types with a softmax activation function yielding the respective plurality of calculated cell type fractions summing to one, and the context-free model comprises 10,000 trainable parameters.

### BRIEF DESCRIPTION OF THE DRAWINGS

FIG. 1A provides a flowchart summarizing stage of data collection, preprocessing, and integration as part of a training data generation process in accordance with an embodiment of the present disclosure.

FIG. 1B provides an overview of metrics for indexed datasets in accordance with an embodiment of the present disclosure.

FIG. 1C provides a detailed outline of a data ingestion and post-processing engine in accordance with an embodiment of the present disclosure.

FIG. 1D illustrates the architecture of a context-free model in accordance with an embodiment of the present disclosure.

FIG. 1E illustrates performance of a context-free model as measured for different training dataset sizes in accordance with an embodiment of the present disclosure.

FIG. 1F illustrates the intuition behind integrated gradients for cell type prediction using Deep Neural Networks in which the demonstrative lineplot showing effect of increasing value of input gene, CD14, from a zero-like baseline to its final value as given in a particular sample, with respect to the final output of model prediction probability for a given celltype, monocytes in accordance with an embodiment of the present disclosure.

FIG. 1G illustrates an exponential curve fitted to four peak performance measures for the disclosed models trained on varying number of training samples to elucidate a theoretical limit of performance given the addition of more training samples in accordance with an embodiment of the present disclosure.

FIG. 2A illustrates a UMAP reduction of peripheral blood mononuclear cells (PBMCSs) in accordance with an embodiment of the present disclosure.

FIG. 2B illustrates deconvolution performance across all cell types for 500 pseudo-mixtures of 5 unique cells types per mixture, as measured by a concordance correlation coefficient (CCC) and Pearsons's R correlation accordance with an embodiment of the present disclosure.

FIG. 2C illustrates a comparison of UniCell deconvolution performance against comparable deconvolution approaches.

FIG. 2D illustrates deconvolution performance stratified by cell type in accordance with an embodiment of the present disclosure.

FIG. 2E illustrates Unicell deconvolution sensitivity against mixture hyperparameters in which the dark blue line denotes mean performance across all cell types, with the light blue shading representing the 95% confidence interval in accordance with an embodiment of the present disclosure.

FIG. 2F illustrates validation of manual cell type annotations using canonical marker genes grouped by annotated cell types in accordance with an embodiment of the present disclosure.

FIG. 3A illustrates a general analysis workflow and process stages for deconvolution of human kidney undergoing ischemic reperfusion injury in accordance with an embodiment of the present disclosure.

FIG. 3B illustrates an overview of kidney anatomy and spatial location of cell types in the kidney in accordance with an embodiment of the present disclosure.

FIG. 3C illustrates spatial deconvolution of select cell types of the kidney across different times points post ischemia reperfusion injury in accordance with an embodiment of the present disclosure.

FIG. 3D illustrates quantified cell type fraction estimates between time point in accordance with an embodiment of the present disclosure.

FIG. 3E illustrates estimates of fibrotic and immune infiltrate before and after ischemia reperfusion injury in accordance with an embodiment of the present disclosure.

FIG. 3F illustrates feature attribution weights for control, pre-treatment kidney in accordance with an embodiment of the present disclosure.

FIG. 3G illustrates (top) changes in feature attribution weights for proximal convoluted tubule (PCT) epithelial cells across time points (bottom) and feature attribute weights for fibrous and immune cell types at 6 weeks post ischemia reperfusion injury in accordance with an embodiment of the present disclosure.

FIG. 4A illustrates an H&E section of a breast invasive adenocarcinoma sample with human-derived pathological annotations overlayed.

FIG. 4B illustrates normalized attribution weights for major cell types identified in FIG. 4A in accordance with an embodiment of the present disclosure.

FIG. 4C illustrates an H&E section of a prostate adenocarcinoma with human-derived pathological annotations overlayed.

FIG. 4D illustrates normalized attribution weights for major cell types identified in FIG. 4C in accordance with an embodiment of the present disclosure.

FIG. 4E illustrates an H&E section of a colorectal adenocarcinoma sample.

FIG. 4F illustrates normalized attribution weights for major cell types identified in FIG. 4E in accordance with an embodiment of the present disclosure.

FIG. 4G illustrates prediction of malignant fraction of bulk RNA samples deconvolved from GTEX (presumed normal) shown per-sample (left) and versus TCGA (presumed cancerous, right) in accordance with an embodiment of the present disclosure.

FIG. 4H illustrates normalized prediction accuracy for cancer subtypes based on TCGA bulk RNA-seq samples in accordance with an embodiment of the present disclosure.

FIG. 4I illustrates model performance, in the form of receiver operating curves, for each cancer subtype present in both a dataset of the present disclosure (UniCell Deconvolve) and the TCGA dataset in accordance with an embodiment of the present disclosure.

FIGS. 4J and 4K illustrate a heatmap of z-score normalized gene feature weights indicating positive associations between high expression (column) and probability of predicting corresponding cancer subtype (row), in which top five genes by positive feature attribution weight are shown and a log of cell counts for each primary cancer type are shown on the left sub-axis of FIG. 4J, in accordance with an embodiment of the present disclosure.

FIG. 4L illustrates BRCA spatial distribution of immune cell markers CD3D (T-Cells), CD68 (Macrophages) in addition to Immune Mediator Chemokine CXCL9, in accordance with an embodiment of the present disclosure.

FIG. 4M illustrates the relative fraction of normal immune cells predicted by the disclosed models across TCGA subtypes in which each dot represents a single sample of a given project, in accordance with an embodiment of the present disclosure.

FIGS. 4N, 4O, and 4P illustrate feature attribution weights for certain epithelial cell subtypes predicted by the disclosed models, in accordance with an embodiment of the present disclosure.

FIG. 5A illustrates cell types in lung homeostasis and interstitial pulmonary fibrosis.

FIG. 5B illustrates predicted cell type fractions for key subtypes involved in accordance with an embodiment of the present disclosure.

FIG. 5C illustrates cell types in lung homeostasis and type II diabetes.

FIG. 5D illustrates Type B Pancreatic cell fraction in normal, pre-Diabetes, and Diabetes samples in accordance with an embodiment of the present disclosure.

FIG. 5E illustrates cell types in normal nerves and multiple sclerosis.

FIG. 5F illustrates Oligodentrocyte fraction in healthy, MS normal-looking, MS-Remyelinating, MS inactive, MS chronic active, and MS active subjects in accordance with an embodiment of the present disclosure.

FIG. 5G provide feature attribution weights for certain cell types deconvolved by the disclosed model for idiopathic pulmonary fibrosis/ALI lung datasets in accordance with an embodiment of the present disclosure.

FIG. 5H provide feature attribution weights for certain cell types deconvolved by the disclosed model for Type 2 diabetes datasets in accordance with an embodiment of the present disclosure.

FIG. 5I provide feature attribution weights for certain cell types deconvolved by the disclosed model for multiple sclerosis datasets in accordance with an embodiment of the present disclosure.

FIG. 5J illustrates the associate between disease status, age, and bet cell fraction in Type 2 Diabetes in accordance with an embodiment of the present disclosure.

FIG. 6A provides an overview of a sample collection process for non-small cell lung cancer tissue collection in accordance with an embodiment of the present disclosure.

FIG. 6B illustrates a UMAP plot of integrated with non-small cell lung cancer scRNA-Seq data colored by biopsy and unbiased clustering status in accordance with an embodiment of the present disclosure.

FIG. 6C illustrates final cell type annotated applied by a model, in accordance with the present disclosure, for each cluster.

FIGS. 6D and 6E illustrate identification of tumor versus normal epithelial cell subsets in accordance with an embodiment of the present disclosure.

FIG. 6F illustrates feature attribution weights for identification of lung adenocarcinoma cancer subtype in accordance with an embodiment of the present disclosure.

FIG. 6G illustrates a heat map of hierarchically ordered cell type predictions by leiden cluster in accordance with an embodiment of the present disclosure.

FIG. 6H illustrates a map of estimated chromosomal copy number aberrations across epithelial cell subsets, using multiple stromal cell types including smooth muscle, endothelial cells, and lung ciliated cells as reference controls in accordance with an embodiment of the present disclosure.

FIG. 6I illustrates a UMAP of epithelial cells colored by CNV-based clusters and absolute CNV scores as well as a scatterplot highlighting correlation between predicted malignant cell status from a model in accordance with the present disclosure and absolute CNV score in accordance with an embodiment of the present disclosure.

FIGS. 6J and 6K illustrate feature attribution weights for key epithelial cell subtypes predicted by a model in accordance with the present disclosure

FIG. 7 illustrates a computer system that makes use of a context-free model to determine a plurality of cell type fractions in a sample in accordance with an embodiment of the present disclosure.

FIGS. 8A, 8B, 8C, 8D, and 8E illustrate methods for use of a context-free model to determine a plurality of cell type fractions in a sample in accordance with an embodiment of the present disclosure, in which optional steps are indicated by dashed boxes, in accordance with an embodiment of the present disclosure.

### DETAILED DESCRIPTION

Systems and methods for training a context-free model to determine cell type fractions are provided. A training set is obtained comprising, for each of a plurality of data stores, for respective each cell represented in the data store, a dataset comprising abundance values for cellular constituents associated with the respective cell. Pseudobulk training mixtures are formed from the training set. For each training mixture, the abundance value for each cellular constituent is averaged across abundance datasets of the cells represented by the respective training mixture thereby forming an averaged abundance dataset for the mixture. For each mixture, the corresponding averaged abundance dataset is inputted into the model thereby obtaining a respective plurality of calculated cell type fractions, each fraction for a different cell type in the training mixture. Model parameters are adjusted based on differences between calculated cell type fractions and mixture fraction ratios for each unique cell type in the respective mixture. Once trained, the model is used to calculate cell fraction ratios of samples for which the cell fraction ratios are not known.

In some embodiment the trained model is an interpretable, deep learning model that deconvolve cell type fractions and predicts cell identity across spatial transcriptomic, bulk-RNA-Seq, and scRNA-Seq datasets without contextualized reference data. In some embodiments the disclosed model is trained on 10 million pseudo-mixtures from the world's largest fully-integrated scRNA-Seq training database comprising 28 million annotated single cells spanning 840 unique cell types from 899 studies. The disclosed model achieves comparable performance on in-silico mixture deconvolution to existing, reference-based, state-of-the-art methods. Data is provided that shows that the disclosed model performs cell type deconvolution with feature attribute analysis that uncovers gene signatures associated with cell-type specific inflammatory-fibrotic responses in ischemic kidney injury, discerns cancer subtypes, and accurately deconvolves tumor microenvironments. The disclosed model identifies pathologic changes in cell fractions among bulk-RNA-Seq data for several disease states. Applied to novel lung cancer scRNA-Seq data, the disclosed model annotates and distinguishes normal from cancerous cells. Overall, the disclosed model enhances transcriptomic data analysis, aiding in assessment of both cellular and spatial context.

Unless defined otherwise, all technical and scientific terms used herein have the same meaning as is commonly understood by one of skill in the art to which this disclosure belongs. All patents and publications referred to herein are incorporated by reference in their entireties.

### Definitions

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, or from 0% to 10%, or from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

As used interchangeably herein, the term “classifier” or “model” refers to a machine learning model or algorithm.

In some embodiments, a classifier is supervised machine learning. Nonlimiting examples of supervised learning algorithms include, but are not limited to, logistic regression, neural networks, support vector machines, Naive Bayes algorithms, nearest neighbor algorithms, random forest algorithms, decision tree algorithms, boosted trees algorithms, multinomial logistic regression algorithms, linear models, linear regression, GradientBoosting, mixture models, hidden Markov models, Gaussian NB algorithms, linear discriminant analysis, or any combinations thereof. In some embodiments, a classifier is a multinomial classifier algorithm. In some embodiments, a classifier is a 2-stage stochastic gradient descent (SGD) model. In some embodiments, a classifier is a deep neural network (e.g., a deep-and-wide sample-level classifier).

Neural networks. In some embodiments, the classifier is a neural network (e.g., a convolutional neural network and/or a residual neural network). Neural network algorithms, also known as artificial neural networks (ANNs), include convolutional and/or residual neural network algorithms (deep learning algorithms). Neural networks can be machine learning algorithms that may be trained to map an input data set to an output data set, where the neural network comprises an interconnected group of nodes organized into multiple layers of nodes. For example, the neural network architecture may comprise at least an input layer, one or more hidden layers, and an output layer. The neural network may comprise any total number of layers, and any number of hidden layers, where the hidden layers function as trainable feature extractors that allow mapping of a set of input data to an output value or set of output values. As used herein, a deep learning algorithm (DNN) can be a neural network comprising a plurality of hidden layers, e.g., two or more hidden layers. Each layer of the neural network can comprise a number of nodes (or “neurons”). A node can receive input that comes either directly from the input data or the output of nodes in previous layers, and perform a specific operation, e.g., a summation operation. In some embodiments, a connection from an input to a node is associated with a parameter (e.g., a weight and/or weighting factor). In some embodiments, the node may sum up the products of all pairs of inputs, xi, and their associated parameters. In some embodiments, the weighted sum is offset with a bias, b. In some embodiments, the output of a node or neuron may be gated using a threshold or activation function, f, which may be a linear or non-linear function. The activation function may be, for example, a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training data set and a gradient descent or backward propagation method so that the output value(s) that the ANN computes are consistent with the examples included in the training data set. The parameters may be obtained from a back propagation neural network training process.

Any of a variety of neural networks may be suitable for use in accordance with the present disclosure. Examples can include, but are not limited to, feedforward neural networks, radial basis function networks, recurrent neural networks, residual neural networks, convolutional neural networks, residual convolutional neural networks, and the like, or any combination thereof. In some embodiments, the machine learning makes use of a pre-trained and/or transfer-learned ANN or deep learning architecture. Convolutional and/or residual neural networks can be used in accordance with the present disclosure.

For instance, a deep neural network classifier comprises an input layer, a plurality of individually parameterized (e.g., weighted) convolutional layers, and an output scorer. The parameters (e.g., weights) of each of the convolutional layers as well as the input layer contribute to the plurality of parameters (e.g., weights) associated with the deep neural network classifier. In some embodiments, at least 100 parameters, at least 1000 parameters, at least 2000 parameters or at least 5000 parameters are associated with the deep neural network classifier. As such, deep neural network classifiers require a computer to be used because they cannot be mentally solved. In other words, given an input to the classifier, the classifier output needs to be determined using a computer rather than mentally in such embodiments. See, for example, Krizhevsky et al., 2012, “Imagenet classification with deep convolutional neural networks,” in Advances in Neural Information Processing Systems 2, Pereira, Burges, Bottou, Weinberger, eds., pp. 1097-1105, Curran Associates, Inc.; Zeiler, 2012 “ADADELTA: an adaptive learning rate method,” CoRR, vol. abs/1212.5701; and Rumelhart et al., 1988, “Neurocomputing: Foundations of research,” ch. Learning Representations by Back-propagating Errors, pp. 696-699, Cambridge, MA, USA: MIT Press, each of which is hereby incorporated by reference.

Neural network algorithms, including convolutional neural network algorithms, suitable for use as classifiers are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. Additional example neural networks suitable for use as classifiers are disclosed in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety. Additional example neural networks suitable for use as classifiers are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, New York, each of which is hereby incorporated by reference in its entirety.

Support vector machines. In some embodiments, the classifier is a support vector machine (SVM). SVM algorithms suitable for use as classifiers are described in, for example, Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For cases in which no linear separation is possible, SVMs can work in combination with the technique of kernels', which automatically realizes a non-linear mapping to a feature space. The hyper-plane found by the SVM in feature space can correspond to a non-linear decision boundary in the input space. In some embodiments, the plurality of parameters (e.g., weights) associated with the SVM define the hyper-plane. In some embodiments, the hyper-plane is defined by at least 10, at least 20, at least 50, or at least 100 parameters and the SVM classifier requires a computer to calculate because it cannot be mentally solved.

Naïve Bayes algorithms. In some embodiments, the classifier is a Naive Bayes algorithm. Naïve Bayes classifiers suitable for use as classifiers are disclosed, for example, in Ng et al., 2002, “On discriminative vs. generative classifiers: A comparison of logistic regression and naive Bayes,” Advances in Neural Information Processing Systems, 14, which is hereby incorporated by reference. A Naive Bayes classifier is any classifier in a family of “probabilistic classifiers” based on applying Bayes' theorem with strong (naïve) independence assumptions between the features. In some embodiments, they are coupled with Kernel density estimation. See, for example, Hastie et al., 2001, The elements of statistical learning: data mining, inference, and prediction, eds. Tibshirani and Friedman, Springer, New York, which is hereby incorporated by reference.

Nearest neighbor algorithms. In some embodiments, a classifier is a nearest neighbor algorithm. Nearest neighbor classifiers can be memory-based and include no classifier to be fit. For nearest neighbors, given a query point x₀ (a test subject), the k training points X₍ᵣ₎, r, . . . , k (here the training subjects) closest in distance to x₀ are identified and then the point x₀ is classified using the k nearest neighbors. In some embodiments, Euclidean distance in feature space is used to determine distance as d₍ᵢ₎=∥x₍ᵢ₎−x₍₀₎∥. Typically, when the nearest neighbor algorithm is used, the abundance data used to compute the linear discriminant is standardized to have mean zero and variance 1. The nearest neighbor rule can be refined to address issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, New York, each of which is hereby incorporated by reference.

A k-nearest neighbor classifier is a non-parametric machine learning method in which the input consists of the k closest training examples in feature space. The output is a class membership. An object is classified by a plurality vote of its neighbors, with the object being assigned to the class most common among its k nearest neighbors (k is a positive integer, typically small). If k=1, then the object is simply assigned to the class of that single nearest neighbor. See, Duda et al., 2001, Pattern Classification, Second Edition, John Wiley & Sons, which is hereby incorporated by reference. In some embodiments, the number of distance calculations needed to solve the k-nearest neighbor classifier is such that a computer is used to solve the classifier for a given input because it cannot be mentally performed.

Random forest, decision tree, and boosted tree algorithms. In some embodiments, the classifier is a decision tree. Decision trees suitable for use as classifiers are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety. In some embodiments, the decision tree classifier includes at least 10, at least 20, at least 50, or at least 100 parameters (e.g., weights and/or decisions) and requires a computer to calculate because it cannot be mentally solved.

Regression. In some embodiments, the classifier uses a regression algorithm. A regression algorithm can be any type of regression. For example, in some embodiments, the regression algorithm is logistic regression. In some embodiments, the regression algorithm is logistic regression with lasso, L2 or elastic net regularization. In some embodiments, those extracted features that have a corresponding regression coefficient that fails to satisfy a threshold value are pruned (removed from) consideration. In some embodiments, a generalization of the logistic regression model that handles multicategory responses is used as the classifier. Logistic regression algorithms are disclosed in Agresti, An Introduction to Categorical Data Analysis, 1996, Chapter 5, pp. 103-144, John Wiley & Son, New York, which is hereby incorporated by reference. In some embodiments, the classifier makes use of a regression model disclosed in Hastie et al., 2001, The Elements of Statistical Learning, Springer-Verlag, New York. In some embodiments, the logistic regression classifier includes at least 10, at least 20, at least 50, at least 100, or at least 1000 parameters (e.g., weights) and requires a computer to calculate because it cannot be mentally solved.

Linear discriminant analysis algorithms. Linear discriminant analysis (LDA), normal discriminant analysis (NDA), or discriminant function analysis can be a generalization of Fisher's linear discriminant, a method used in statistics, pattern recognition, and machine learning to find a linear combination of features that characterizes or separates two or more classes of objects or events. The resulting combination can be used as the classifier (linear classifier) in some embodiments of the present disclosure.

Mixture model and Hidden Markov model. In some embodiments, the classifier is a mixture model, such as that described in Mclachlan et al., Bioinformatics 18(3): 413-422, 2002. In some embodiments, in particular, those embodiments including a temporal component, the classifier is a hidden Markov model such as described by Schliep et al., 2003, Bioinformatics 19(1):i255-i263.

Clustering. In some embodiments, the classifier is an unsupervised clustering model. In some embodiments, the classifier is a supervised clustering model. Clustering algorithms suitable for use as classifiers are described, for example, at pages 211-256 of Duda and Hart, Pattern Classification and Scene Analysis, 1973, John Wiley & Sons, Inc., New York, (hereinafter “Duda 1973”) which is hereby incorporated by reference in its entirety. The clustering problem can be described as one of finding natural groupings in a dataset. To identify natural groupings, two issues can be addressed. First, a way to measure similarity (or dissimilarity) between two samples can be determined. This metric (e.g., similarity measure) can be used to ensure that the samples in one cluster are more like one another than they are to samples in other clusters. Second, a mechanism for partitioning the data into clusters using the similarity measure can be determined. One way to begin a clustering investigation can be to define a distance function and to compute the matrix of distances between all pairs of samples in the training set. If distance is a good measure of similarity, then the distance between reference entities in the same cluster can be significantly less than the distance between the reference entities in different clusters. However, clustering may not use a distance metric. For example, a nonmetric similarity function s(x, x′) can be used to compare two vectors x and x′. s(x, x′) can be a symmetric function whose value is large when x and x′ are somehow “similar.” Once a method for measuring “similarity” or “dissimilarity” between points in a dataset has been selected, clustering can use a criterion function that measures the clustering quality of any partition of the data. Partitions of the data set that extremize the criterion function can be used to cluster the data. Particular exemplary clustering techniques that can be used in the present disclosure can include, but are not limited to, hierarchical clustering (agglomerative clustering using a nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering (e.g., with no preconceived number of clusters and/or no predetermination of cluster assignments).

Ensembles of classifiers and boosting. In some embodiments, an ensemble (two or more) of classifiers is used. In some embodiments, a boosting technique such as AdaBoost is used in conjunction with many other types of learning algorithms to improve the performance of the classifier. In this approach, the output of any of the classifiers disclosed herein, or their equivalents, is combined into a weighted sum that represents the final output of the boosted classifier. In some embodiments, the plurality of outputs from the classifiers is combined using any measure of central tendency known in the art, including but not limited to a mean, median, mode, a weighted mean, weighted median, weighted mode, etc. In some embodiments, the plurality of outputs is combined using a voting method. In some embodiments, a respective classifier in the ensemble of classifiers is weighted or unweighted.

As used herein, the term “parameter” refers to any coefficient or, similarly, any value of an internal or external element (e.g., a weight and/or a hyperparameter) in an algorithm, model, regressor, and/or classifier that can affect (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that can be used to control, modify, tailor, and/or adjust the behavior, learning, and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some embodiments, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods). In some embodiments, an algorithm, model, regressor, and/or classifier of the present disclosure includes a plurality of parameters. In some embodiments, the plurality of parameters is n parameters, where: n≥2; n≥5; n≥10; n≥25; n≥40; n≥50; n≥75; n≥100; n≥125; n≥150; n≥200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×10⁶, n≥5×10⁶, or n≥1×10⁷. As such, the algorithms, models, regressors, and/or classifiers of the present disclosure cannot be mentally performed. In some embodiments n is between 10,000 and 1×10⁷, between 100,000 and 5×10⁶, or between 500,000 and 1×10⁶. In some embodiments, the algorithms, models, regressors, and/or classifier of the present disclosure operate in a k-dimensional space, where k is a positive integer of 5 or greater (e.g., 5, 6, 7, 8, 9, 10, etc.). As such, the algorithms, models, regressors, and/or classifiers of the present disclosure cannot be mentally performed.

As used herein, the term “untrained model” (e.g., “untrained classifier” and/or “untrained neural network”) refers to a machine learning model or algorithm, such as a classifier or a neural network, that has not been trained on a target dataset. In some embodiments, “training a model” (e.g., “training a neural network”) refers to the process of training an untrained or partially trained model (e.g., “an untrained or partially trained neural network”). Moreover, it will be appreciated that the term “untrained model” does not exclude the possibility that transfer learning techniques are used in such training of the untrained or partially trained model. For instance, Fernandes et al., 2017, “Transfer Learning with Partial Observability Applied to Cervical Cancer Screening,” Pattern Recognition and Image Analysis: 8^{th} Iberian Conference Proceedings, 243-250, which is hereby incorporated by reference, provides non-limiting examples of such transfer learning. In instances where transfer learning is used, the untrained classifier described above is provided with additional data over and beyond that of the primary training dataset. Typically, this additional data is in the form of parameters (e.g., coefficients, weights, and/or hyperparameters) that were learned from another, auxiliary training dataset. Moreover, while a description of a single auxiliary training dataset has been disclosed, it will be appreciated that there is no limit on the number of auxiliary training datasets that may be used to complement the primary training dataset in training the untrained model in the present disclosure. For instance, in some embodiments, two or more auxiliary training datasets, three or more auxiliary training datasets, four or more auxiliary training datasets or five or more auxiliary training datasets are used to complement the primary training dataset through transfer learning, where each such auxiliary dataset is different than the primary training dataset. Any manner of transfer learning may be used in such embodiments. For instance, consider the case where there is a first auxiliary training dataset and a second auxiliary training dataset in addition to the primary training dataset. The parameters learned from the first auxiliary training dataset (by application of a first classifier to the first auxiliary training dataset) may be applied to the second auxiliary training dataset using transfer learning techniques (e.g., a second classifier that is the same or different from the first classifier), which in turn may result in a trained intermediate classifier whose parameters are then applied to the primary training dataset and this, in conjunction with the primary training dataset itself, is applied to the untrained classifier. Alternatively, a first set of parameters learned from the first auxiliary training dataset (by application of a first classifier to the first auxiliary training dataset) and a second set of parameters learned from the second auxiliary training dataset (by application of a second classifier that is the same or different from the first classifier to the second auxiliary training dataset) may each individually be applied to a separate instance of the primary training dataset (e.g., by separate independent matrix multiplications) and both such applications of the parameters to separate instances of the primary training dataset in conjunction with the primary training dataset itself (or some reduced form of the primary training dataset such as principal components or regression coefficients learned from the primary training set) may then be applied to the untrained classifier in order to train the untrained classifier.

For the avoidance of doubt, it is intended herein that particular features (for example integers, characteristics, values, uses, diseases, formulae, compounds or groups) described in conjunction with a particular aspect, embodiment or example of the disclosure are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The disclosure is not restricted to any details of any disclosed embodiments. The disclosure extends to any novel one, or novel combination, of the features disclosed in this specification (including any accompanying claims, abstract and drawings), or to any novel one, or any novel combination, of the steps of any method or process so disclosed.

Moreover, as used herein, the term “about” means that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All embodiments of the invention can, in the alternative, be more specifically defined by any of the transitional terms “comprising,” “consisting essentially of,” and “consisting of.”

### Example System Diagram

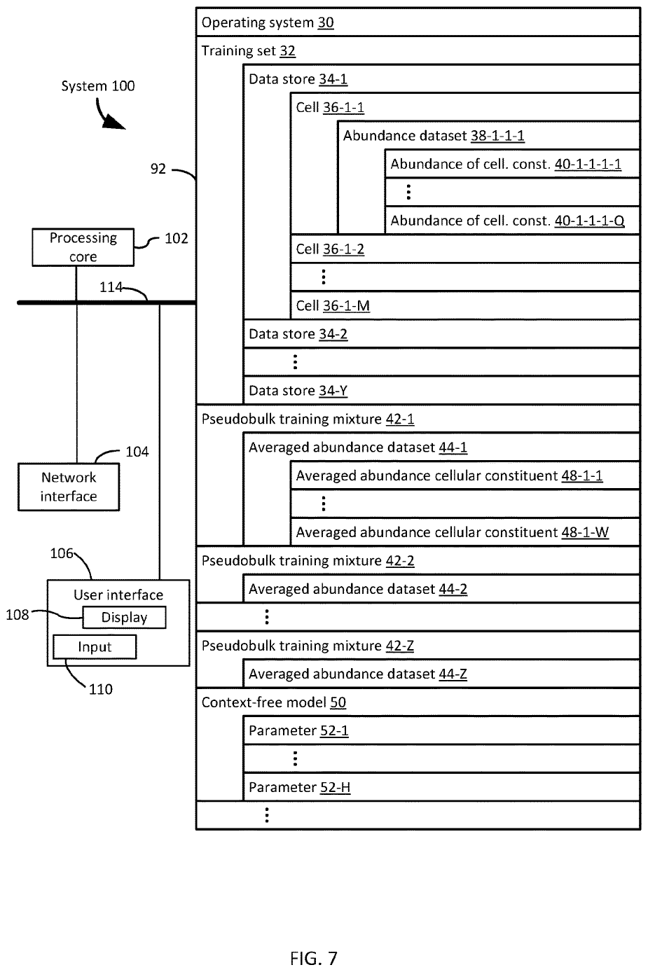

FIG. 7 illustrates a computer system 700 for training a context-free model to determine a plurality of cell type fractions in a sample. In typical embodiments, computer system 100 comprises one or more computers. For purposes of illustration in FIG. 7, the computer system 100 is represented as a single computer that includes all of the functionality of the disclosed computer system 100. However, the present disclosure is not so limited. The functionality of the computer system 100 may be spread across any number of networked computers and/or reside on each of several networked computers and/or virtual machines. One of skill in the art will appreciate that a wide array of different computer topologies are possible for the computer system 100 and all such topologies are within the scope of the present disclosure.

Turning to FIG. 7 with the foregoing in mind, the computer system 100 comprises one or more processing units (CPUs) 102, a network or other communications interface 104, a user interface 106 (e.g., including an optional display 108 and optional keyboard 110 or other form of input device), a memory 92 (e.g., random access memory, persistent memory, or combination thereof), and one or more communication busses 114 for interconnecting the aforementioned components. To the extent that components of memory 92 are not persistent, data in memory 92 can be seamlessly shared with non-volatile memory (not shown) or portions of memory 92 that are non-volatile/persistent using known computing techniques such as caching. Memory 92 can include mass storage that is remotely located with respect to the central processing unit(s) 102. In other words, some data stored in memory 92 may in fact be hosted on computers that are external to computer system 100 but that can be electronically accessed by the computer system 100 over an Internet, intranet, or other form of network or electronic cable using network interface 104. In some embodiments, the computer system 100 makes use of models that are run from the memory associated with one or more graphical processing units in order to improve the speed and performance of the system. In some alternative embodiments, the computer system 100 makes use of models that are run from memory 92 rather than memory associated with a graphical processing unit.

The memory 92 of the computer system 100 stores:
an optional operating system 30 that includes procedures for handling various basic system services;
a training set 32 comprising, for each respective data store 34 in plurality of data stores (34-1, 34-2, . . . , 34-Y), for each respective cell 36 in a respective population of cells 36-1-1, 36-1-2, . . . , 36-1-M, . . . ) represented in the respective data store, a respective abundance dataset 38 comprising a respective abundance value for each respective cellular constituent in a corresponding plurality of cellular constituents associated with the respective cell;
a plurality of pseudobulk training mixtures 42-1, 42-2, . . . , 42-Z formed from the training set 32, were each respective pseudobulk training mixture 42 comprises an averaged abundance dataset 44 for the respective plurality of cells represented by the respective pseudobulk training mixture in which the respective abundance value for each respective corresponding cellular constituent across each abundance dataset of the respective plurality of cells represented by the respective pseudobulk training mixture are averaged; and
a context-free model for determining a plurality of cell type fractions in a sample, where the context-free model comprises a plurality of parameters 52-1, . . . , 52-H.

In some implementations, one or more of the above identified data elements or modules of the computer system 100 are stored in one or more of the previously mentioned memory devices, and correspond to a set of instructions for performing a function described above. The above identified data, modules or programs (e.g., sets of instructions) need not be implemented as separate software programs, procedures or modules, and thus various subsets of these modules may be combined or otherwise re-arranged in various implementations. In some implementations, the memory 92 optionally stores a subset of the modules and data structures identified above. Furthermore, in some embodiments the memory 92 stores additional modules and data structures not described above.

### Example Methods

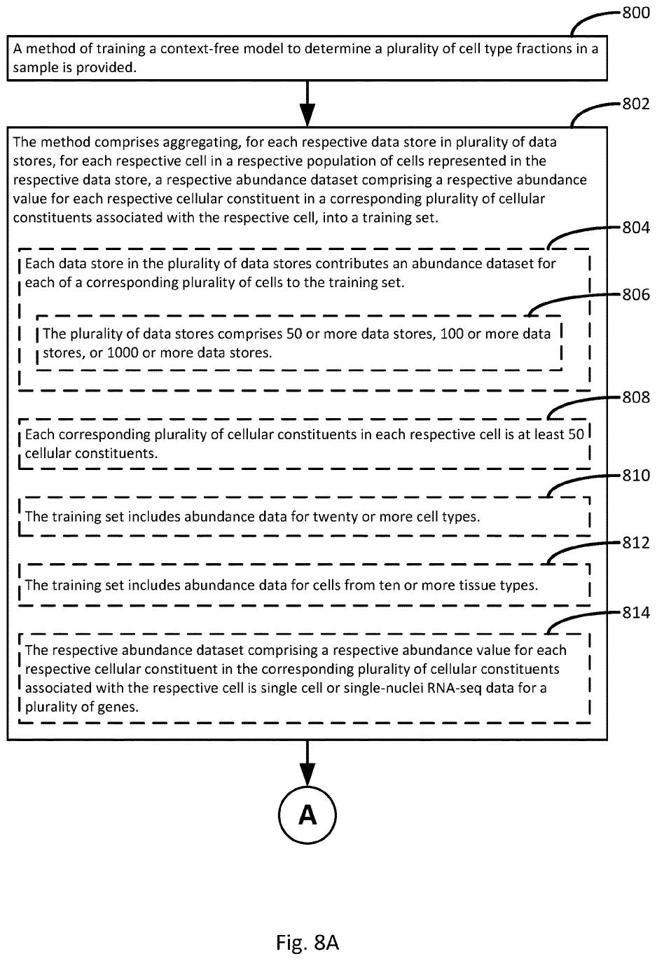

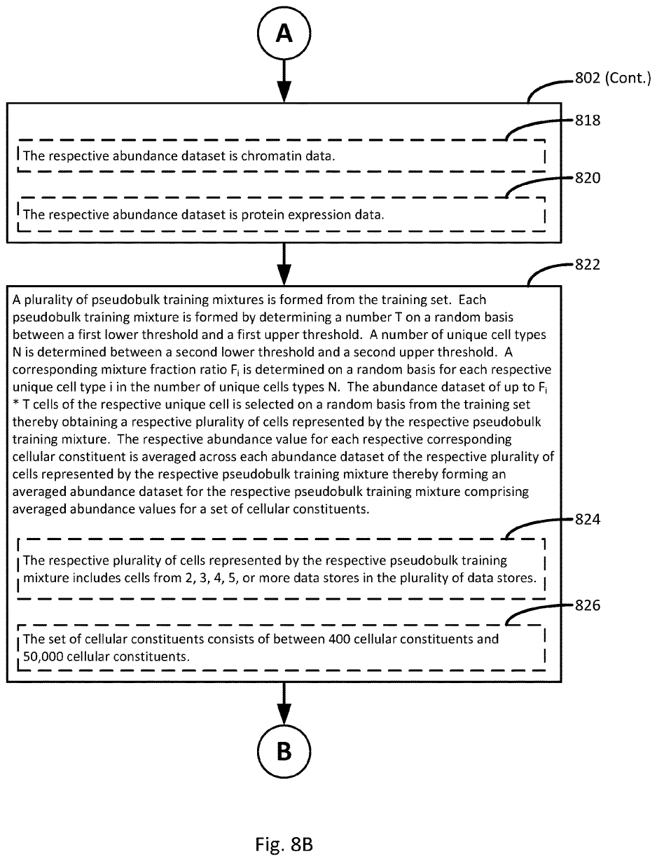

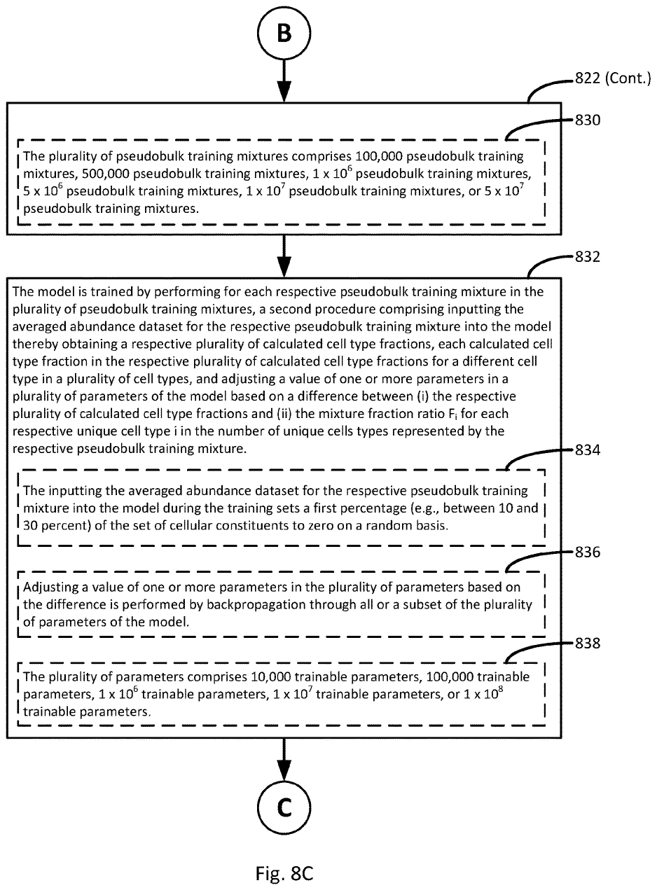

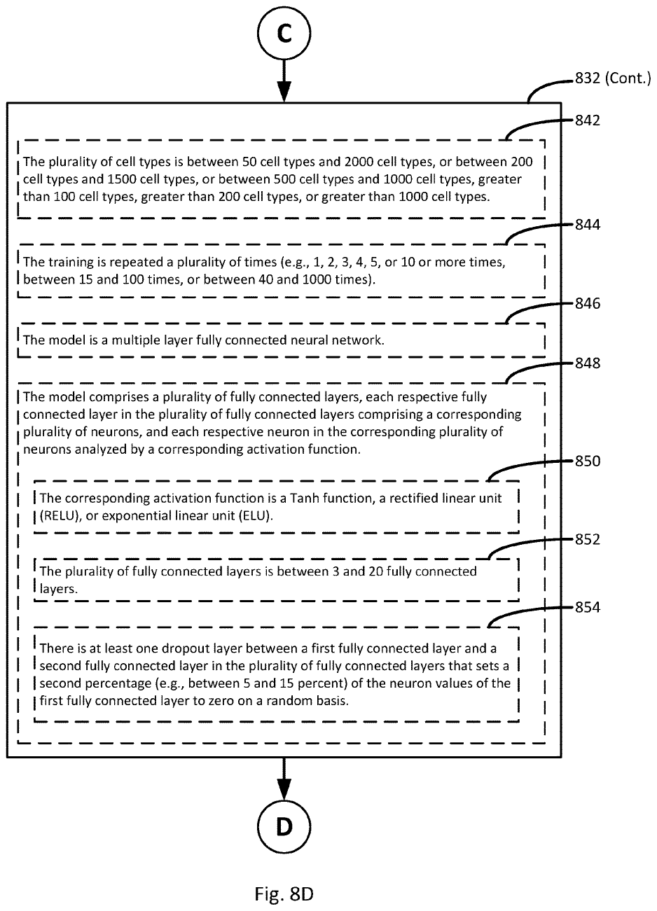

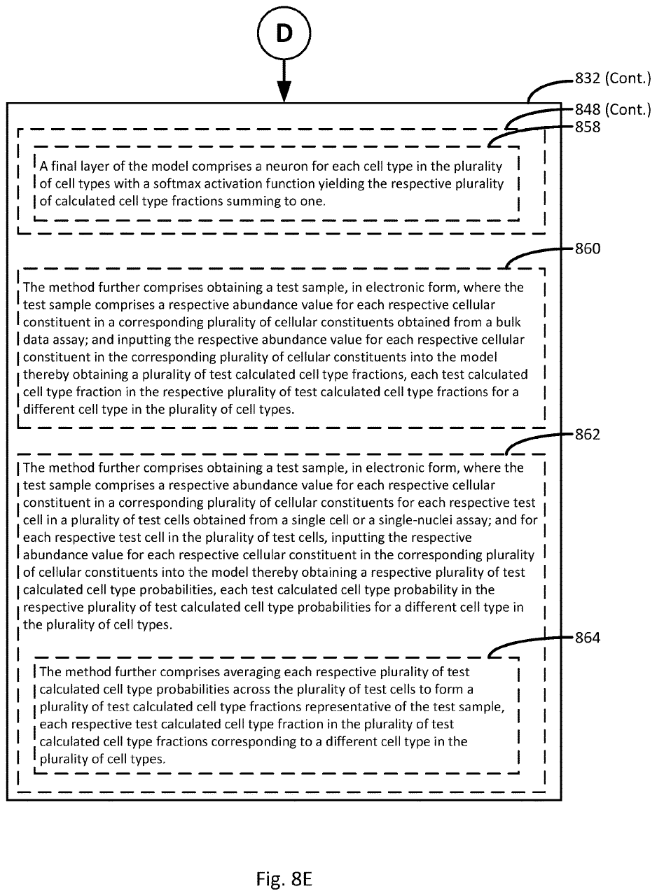

Now that a system for training a context-free model to determine a plurality of cell type fractions in a sample has been disclosed, methods for training a context-free model to determine a plurality of cell type fractions in a sample are detailed with reference to FIG. 8 and discussed below.

Block 800. In accordance with block 700 of FIG. 8A, a method of training a context-free model 50 to determine a plurality of cell type fractions in a sample is provided.

Block 802. The method comprises aggregating, for each respective data store 34 in plurality of data stores, for each respective cell 36 in a respective population of cells represented in the respective data store, a respective abundance dataset 38 comprising a respective abundance value 40 for each respective cellular constituent in a corresponding plurality of cellular constituents associated with the respective cell, into a training set 32. For instance, Example 1 below details how such data can be obtained from NCBI Gene Expression Omnibus (GEO) [29] and EMBL ArrayExpress (AE) [30], as well as numerous secondary sources including the UCSC Cell Browser [31], EMBL-EBI Single Cell Expression Atlas [32], TISCH [33], and the CZI Human Cell Atlas [34]. For primary data repositories GEO/AE, an API-based programmatic keyword search was performed for “scRNA-Seq OR single cell OR single-cell sequencing OR scRNA” to collect an exhaustive list of studies potentially containing scRNA-Seq data. Primary and secondary sources can then be manually cross-referenced to eliminate duplicate entries.

Blocks 804-806. Each data store 34 in the plurality of data stores 34 contributes an abundance dataset 38 for each of a corresponding plurality of cells 36 to the training set (block 804). In some embodiments, the plurality of data stores comprises 50 or more data stores 34, 100 or more data stores 34, or 1000 or more data stores 34. However, in other embodiments fewer data stores are used, such as just as single data store, two data stores, or between 3 and 50 data stores.

Block 808. Referring to block 808, in some embodiments each corresponding plurality of cellular constituents in each respective cell 36 is at least 50 cellular constituents. In some embodiments each cellular constituent is gene expression data. In some embodiments each of the cellular constituents are any of the single cell data described in Example 1. In some embodiments, each corresponding plurality of cellular constituents consists of all or a subset of the coding or non-coding human genes. In some embodiments, each corresponding plurality of cellular constituents consists between 50 and 100, between 25 and 500, between 100 and 1000, more than 2000, more than 3000, between 2000 and 10,000 or between 3 and 25,000 coding and/or noncoding human genes.

Block 810 and 812. Referring to block 810, in some embodiments the training set 32 includes abundance data for twenty or more cell types. In some embodiments the training set 32 includes data for few than twenty cells types such as 2, 3, 4, 5, 6, 7, 8, 9, 10, 11, 12, 13, 14, 15, 16, 17, 18, or 19 cell types. Referring to block 812 of FIG. 8A, in some embodiments the training set 32 includes abundance data for cells from ten or more tissue types. In some embodiments the cells types are all or any subset of the cell types found in any one or any combination of the databases references in Example 1.

Block 814. Referring to block 814 of FIG. 8A, in some embodiments the respective abundance dataset 38 comprising a respective abundance value for each respective cellular constituent in the corresponding plurality of cellular constituents associated with the respective cell is single cell or single-nuclei RNA-seq data for a plurality of genes. In some embodiments these abundance values are normalized, for instance using any combination of the normalization techniques described in Examples 1, 2, 4, or 6.

Block 818. Referring to block 818 of FIG. 8B, in some embodiments the respective abundance dataset 38 is chromatin (accessibility) data. See, for example, Tsompana and Buck, 2014, “Chromatin accessibility: a window into the genome,” Epigenetics & Chromatin 7 (33), which is hereby incorporated by reference, for a description of chromatin accessibility data.

Block 820. Referring to block 820 of FIG. 8B, in some embodiments the respective abundance dataset 38 is protein expression data.

Block 822-830. Referring to block 822 of FIG. 8B, a plurality of pseudobulk training mixtures 42 is formed from the training set 32. Each pseudobulk training mixture 42 is formed by determining a number T on a random basis between a first lower threshold and a first upper threshold. In some embodiments the first lower threshold is five and the first upper threshold is 100,000. In some embodiments the first lower threshold is between 10 and 50,000 and the first upper threshold is between 100 and 100,000 provided that the first lower threshold is smaller than the first upper threshold.

A number of unique cell types N is determined between a second lower threshold and a second upper threshold. In some embodiments the second lower threshold is five and the second upper threshold is 1000. In some embodiments the second lower threshold is between 2 and 500 and the first upper threshold is between 3 and 5000 provided that the second lower threshold is smaller than the second upper threshold.

A corresponding mixture fraction ratio Fᵢ is determined on a random basis for each respective unique cell type i in the number of unique cells types N. The abundance dataset of up to Fᵢ*T cells of the respective unique cell is selected on a random basis across all the data stores of the training set 32 thereby obtaining a respective plurality of cells representing the respective pseudobulk training mixture 42. The respective abundance value for each respective corresponding cellular constituent acquired from the training set in this manner is averaged across each abundance dataset of the respective plurality of cells represented by the respective pseudobulk training mixture thereby forming an averaged abundance dataset 44 for the respective pseudobulk training mixture comprising averaged abundance values for a set of cellular constituents. See, for example, Example 2 below.

Referring to block 824, in some embodiments the respective plurality of cells represented by the respective pseudobulk training mixture 42 includes cells from 2, 3, 4, 5, or more data stores 34 in the plurality of data stores. Referring to block 826, in some embodiments the set of cellular constituents for an averaged abundance dataset consists of between 400 cellular constituents and 50,000 cellular constituents. Referring to block 830 of FIG. 8C, in some embodiments the plurality of pseudobulk training mixtures comprises 100,000 pseudobulk training mixtures, 500,000 pseudobulk training mixtures, 1×10⁶ pseudobulk training mixtures, 5×10⁶ pseudobulk training mixtures, 1×10⁷ pseudobulk training mixtures, or 5×10⁷ pseudobulk training mixtures.

Blocks 832 838. Referring to block 832 of FIG. 8C, the context-free model 50 is trained by performing, for each respective pseudobulk training mixture 42 in the plurality of pseudobulk training mixtures, a procedure comprising inputting the averaged abundance dataset 48 for the respective pseudobulk training mixture 42 into the context-free model 50 thereby obtaining a respective plurality of calculated cell type fractions, each calculated cell type fraction in the respective plurality of calculated cell type fractions for a different cell type in a plurality of cell types, and adjusting a value of one or more parameters 52 in a plurality of parameters of the model 50 based on a difference between (i) the respective plurality of calculated cell type fractions and (ii) the mixture fraction ratio Fᵢ for each respective unique cell type i in the number of unique cells types represented by the respective pseudobulk training mixture 42. See, for example, Example 2 below.

Referring to block 834, in some embodiments, the inputting the averaged abundance dataset 44 for the respective pseudobulk training mixture 42 into the context-free model 50 during the training, sets an abundance value of a first percentage (e.g., between 10 and 30 percent) of the set of cellular constituents to zero on a random basis.

In some embodiment some amount of percent Gaussian noise is first injected into the normalized expression profile of each cellular constituent (e.g. a normalized expression value of 0.8 from a given cellular constituent i will range anywhere from 0.75-0.85 following 5% gaussian noise injection). In some embodiments the Gaussian noise injection is followed in the model by a dropout layer, where some amount of input values are randomly set to zero. The combination of noise and dropout further encourage the model to learn more complex representations of cell types that are robust to noise and missing cellular constituents. In some embodiments, the amount of Gaussian noise injected is between 1 percent and 10 percent. In some embodiments, the percent of cellular constituents whose values are dropped to zero is between 2% and 30% of the cellular constituents inputted into the model 50.

Referring to block 836, in some embodiments, the adjusting a value of one or more parameters 52 in the plurality of parameters of the context-free model 50 based on the difference is performed by backpropagation through all or a subset of the plurality of parameters 52 of the model 50. For instance, in some embodiments Tensorflow 2.5.0 is used, with the Adam optimizer for supervised backpropagation with a learning rate of 0.0001 and an effective batch size of 256 as discussed below in Example 2.

In another exemplary nonlimiting embodiment, the model is trained against the errors in the calculated cell type fractions made by the model by stochastic gradient descent with the AdaDelta adaptive learning method (Zeiler, 2012 “ADADELTA: an adaptive learning rate method,”’ CoRR, vol. abs/1212.5701, which is hereby incorporated by reference), and the back propagation algorithm provided in Rumelhart et al., 1988, “Neurocomputing: Foundations of research,” ch. Learning Representations by Back-propagating Errors, pp. 696-699, Cambridge, MA, USA: MIT Press, which is hereby incorporated by reference.

Referring to block 838, in some embodiments the plurality of parameters comprises 10,000 trainable parameters, 100,000 trainable parameters, 1×10⁶ trainable parameters, 1×10⁷ trainable parameters, or 1×10⁸ trainable parameters. See also, the definition of parameters given in the definitions section above.

Blocks 842-844. Referring to block 842 on FIG. 8D, in some embodiments the plurality of cell types is between 50 cell types and 2000 cell types, or between 200 cell types and 1500 cell types, or between 500 cell types and 1000 cell types, greater than 100 cell types, greater than 200 cell types, or greater than 1000 cell types. Referring to block 844 on FIG. 8D, in some embodiments the training of block 832 is repeated a plurality of times (e.g., 1, 2, 3, 4, 5, or 10 or more times, between 15 and 100 times, or between 40 and 1000 times).

Blocks 846-858. Referring to block 846 of FIG. 8D, in some embodiments the model 50 is a multiple layer fully connected neural network. Such fully connected neural networks are also known as multilayer perceptrons (MLP). In some embodiments, a MLP is a class of feedforward artificial neural network (ANN) comprising at least three layers of nodes: an input layer, a hidden layer and an output layer. In such embodiments, except for the input nodes, each node is a neuron that uses a nonlinear activation function. More disclosure on suitable MLPs that can serve as model 50 in some embodiments of the present disclosure is found in Vang-mata ed., 2020, Multilayer Perceptrons: Theory and Applications, Nova Science Publishers, Hauppauge, New York, which is hereby incorporated by reference.

Referring to block 848 of FIG. 8D, in some embodiments the model 50 comprises a plurality of fully connected layers, each respective fully connected layer in the plurality of fully connected layers comprising a corresponding plurality of neurons, and each respective neuron in the corresponding plurality of neurons analyzed by a corresponding activation function. In some embodiments, the model consists of between 2 and 50, between 2 and 40, between 2 and 30, or between 5 and 100 fully connected layers. In some embodiments, each fully connected layer consists of between 5 and 5000 neurons, between 10 and 4000 neurons or between 50 and 3000 neurons.

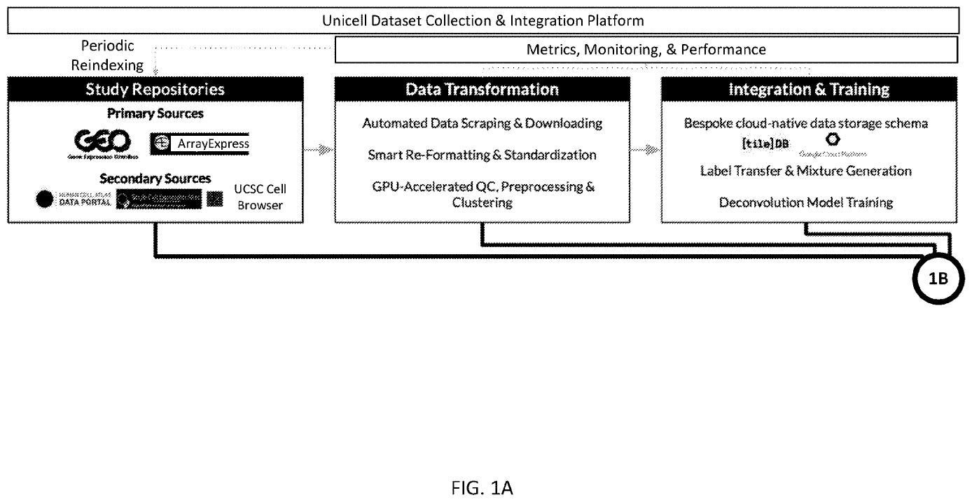

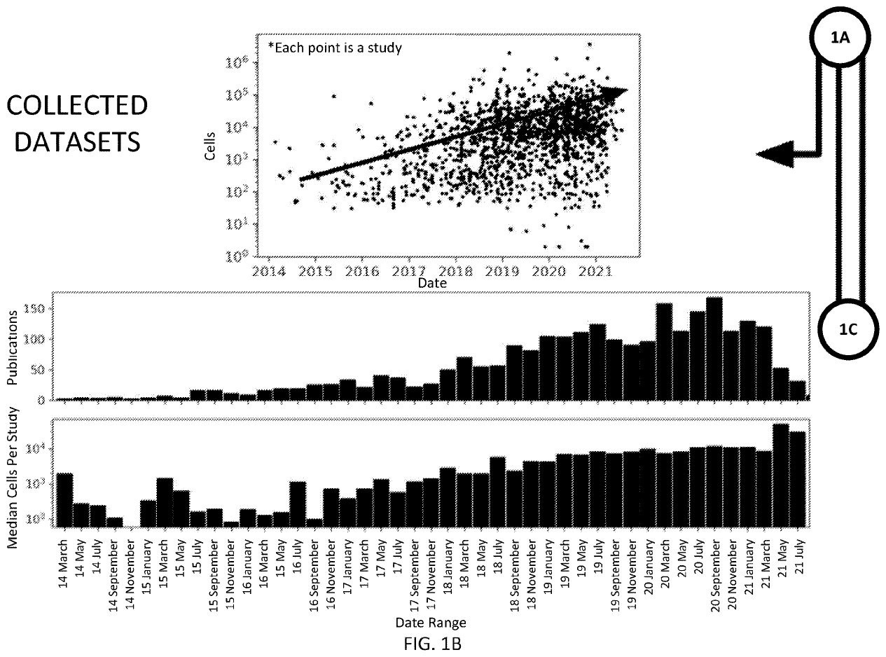

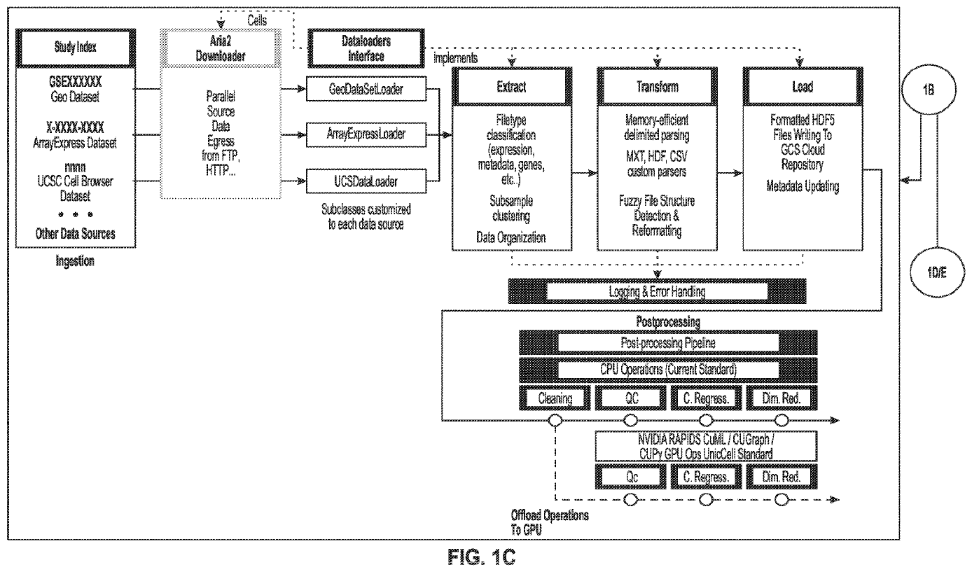

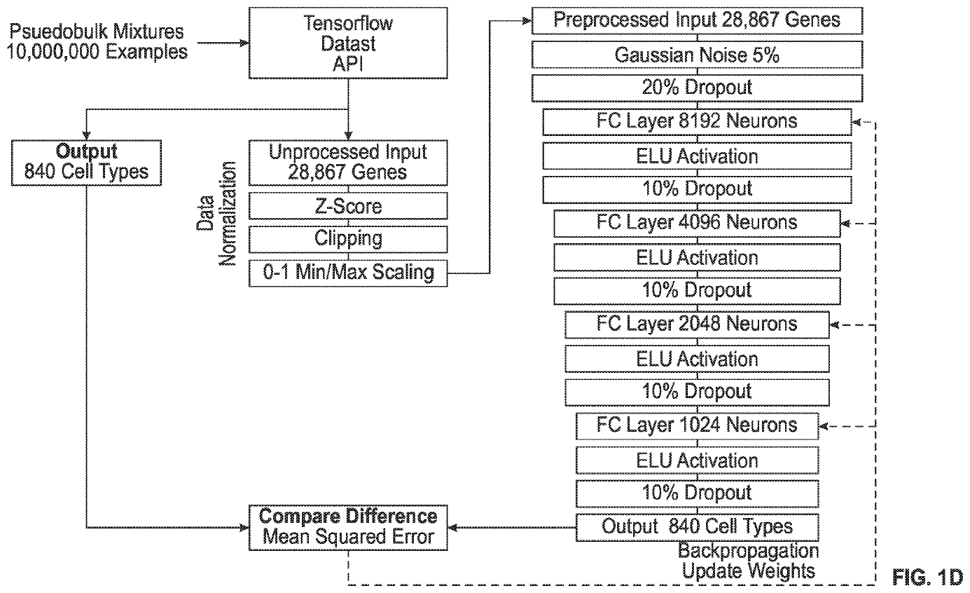

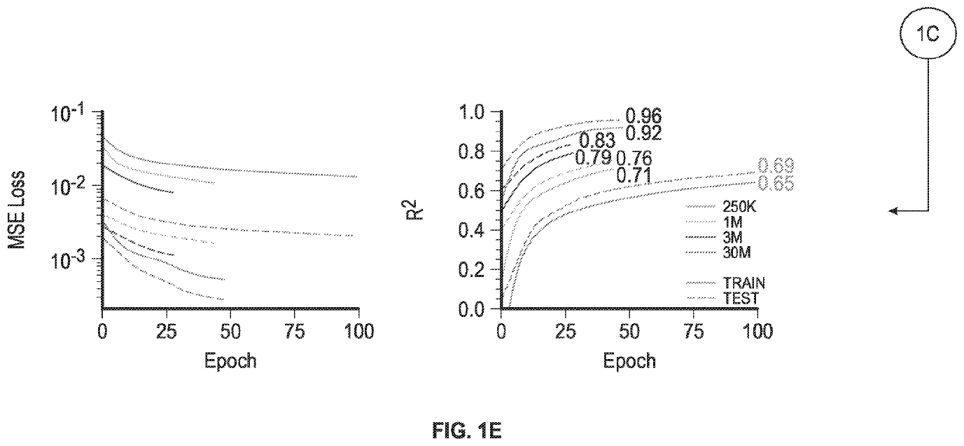

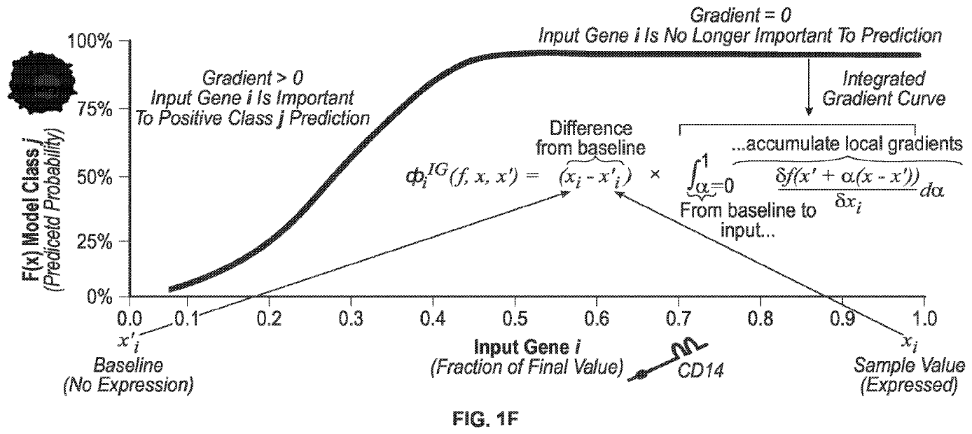

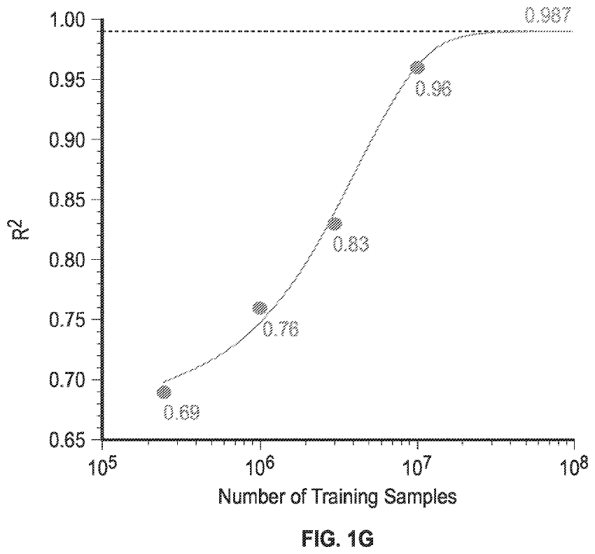

FIG. 1D illustrates and an example of such a model. Referring to block 850 of FIG. 8E, in some embodiments the corresponding activation function is a Tanh function, a rectified linear unit (RELU), or an exponential linear unit (ELU). In some embodiments, the corresponding activation function is Tanh, sigmoid, softmax, Gaussian, Boltzmann-weighted averaging, absolute value, linear, rectified linear unit (ReLU), an exponential linear unit (ELU), a bounded rectified linear, soft rectified linear, parameterized rectified linear, average, max, min, sign, square, square root, multiquadric, inverse quadratic, inverse multiquadric, polyharmonic spline, swish, mish, Gaussian error linear unit (GeLU), and/or thin plate spline.

Referring to block 852 of FIG. 8E, in some embodiments the plurality of fully connected layers is between 3 and 20 fully connected layers. Referring to block 854 of FIG. 8E, in some embodiments there is at least one dropout layer between a first fully connected layer and a second fully connected layer in the plurality of fully connected layers that sets a second percentage (e.g., between 5 and 15 percent) of the neuron values of the first fully connected layer to zero on a random basis. Referring to block 858 on page 8E, in some embodiments a final layer of the model 50 comprises a neuron for each cell type in the plurality of cell types with a softmax activation function yielding the respective plurality of calculated cell type fractions summing to one.

Block 860. Referring to block 860 of FIG. 8E, in some embodiments the method further comprises obtaining a test sample, in electronic form, where the test sample comprises a respective abundance value for each respective cellular constituent in a plurality of cellular constituents obtained from a bulk data assay. In some embodiments, the plurality of cellular constituents consists of between 2 and 50,000 different cellular constituents, between 10 and 40,000 different cellular constituents, between 100 and 30,000 different cellular constituents for between 250 and 25,000 different cellular constituents. In some embodiments the plurality of cellular constituents consists of at least 10, at least 20, at least 30, at least 40, at least 50, at least 60, at least 70, at least 80, at least 90, at least 100, or at least 1000 different cellular constituents. The respective abundance value for each respective cellular constituent in the corresponding plurality of cellular constituents is inputted into the model thereby obtaining a plurality of test calculated cell type fractions, each test calculated cell type fraction in the respective plurality of test calculated cell type fractions for a different cell type in the plurality of cell types. In some embodiments, the plurality of cell types is 2, 3, 4, 5, 6, 7, 8, 9, 10, 11, 12, 13, 14, 15, 16, 17, 18, 19, 20, 21, 22, 23, 23, 25, 26, 27, 28, 29, 30, 31, 32, 33, 34, 35, 36, 37, 38, 39 or 40 different cell types. In some embodiments the plurality of cell types comprises 40, 50, 60, 70, 80, 90, or 100 different cell types. In some embodiments the plurality of cell types is between 10 and 1000 different cell types.

Blocks 862 and 864. Referring to block 862 of FIG. 8E, in some embodiments the method further comprises obtaining a test sample, in electronic form, where the test sample comprises a respective abundance value for each respective cellular constituent in a plurality of cellular constituents for each respective test cell in a plurality of test cells obtained from a single cell or a single-nuclei assay. In some embodiments, the corresponding plurality of cellular constituents consists of between 2 and 50,000 different cellular constituents, between 10 and 40,000 different cellular constituents, between 100 and 30,000 different cellular constituents for between 250 and 25,000 different cellular constituents. In some embodiments the plurality of cellular constituents consists of at least 10, at least 20, at least 30, at least 40, at least 50, at least 60, at least 70, at least 80, at least 90, at least 100, or at least 1000 different cellular constituents. In such embodiments, for each respective test cell in the plurality of test cells, the respective abundance value for each respective cellular constituent in the corresponding plurality of cellular constituents is inputted into the model 50 thereby obtaining a respective plurality of test calculated cell type probabilities, each test calculated cell type probability in the respective plurality of test calculated cell type probabilities for a different cell type in the plurality of cell types. In some embodiments, the plurality of cell types is 2, 3, 4, 5, 6, 7, 8, 9, 10, 11, 12, 13, 14, 15, 16, 17, 18, 19, 20, 21, 22, 23, 23, 25, 26, 27, 28, 29, 30, 31, 32, 33, 34, 35, 36, 37, 38, 39 or 40 different cell types. In some embodiments the plurality of cell types comprises 40, 50, 60, 70, 80, 90, or 100 different cell types. In some embodiments the plurality of cell types is between 10 and 1000 different cell types. Referring to block 864, in some such embodiments the method further comprises averaging each respective plurality of test calculated cell type probabilities across the plurality of test cells to form a plurality of test calculated cell type fractions representative of the test sample, each respective test calculated cell type fraction in the plurality of test calculated cell type fractions corresponding to a different cell type in the plurality of cell types.

### EXAMPLES

The present disclosure addresses the problems identified in the background. One aspect of the present disclosure provides a pre-trained, context-free, model for universal cell type deconvolution. The model was trained using 10 million pseudobulk RNA mixtures generated from the fully integrated scRNA-Seq databases, comprising 28 million fully-annotated single cells representing at least 840 cell types collected from 899 uniformly preprocessed, validated, and published single-cell datasets. The following examples describe the collection and integration strategy used to build training data for model, and also detail the architecture of the model. The examples demonstrate how baseline performance compares favorably to existing reference-based approaches, with feature attribute analysis enabling orthogonal validation of predictions by associating gene expression with particular cell types. The examples highlight the ability of the model to deconvolve changes to immune and stromal cell infiltrates in response to ischemic kidney injury, associating differentially active stress response genes to kidney epithelial cell types. The examples also detail how application of the model to bulk-RNA-Seq data pinpoints specific losses in pancreatic beta cell and oligodendrocyte fractions in type 2 diabetes and multiple sclerosis, respectively. The disclosed model also accurately differentiates between cancer subtypes across bulk, spatial and single-cell data. Lastly, the disclosed examples illustrate how the model was used to annotate novel primary human lung cancer data, providing marker genes to corroborate predictions, and distinguishes normal from cancerous epithelial cells.

### Example 1—Single Cell Dataset Curation

The collection and integration of a large annotated scRNA-Seq database favorably affect the performance of the models of the present disclosure. In this example, the major stages of a data curation process (summarized in FIG. 1A) are described and technical approaches used to overcome challenges inherent to operating with integrated high-dimensional data at scale are highlighted.

Study Indexing. An index of all publicly available scRNA-Seq datasets was generated, leveraging both primary sources such as NCBI Gene Expression Omnibus (GEO) [29] and EMBL ArrayExpress (AE) [30], as well as numerous secondary sources including the UCSC Cell Browser [31], EMBL-EBI Single Cell Expression Atlas [32], TISCH [33], and the CZI Human Cell Atlas [34]. For primary data repositories GEO/AE, an API-based programmatic keyword search was performed for “scRNA-Seq OR single cell OR single-cell sequencing OR scRNA” to collect an exhaustive list of studies potentially containing scRNA-Seq data. Primary and secondary sources were then manually cross-referenced to eliminate duplicate entries.

The base index contains 2,695 studies published between January 2015 and June 2021. Studies published before or after this period were not included in the disclosed context-free model. Examining global trends in publications (see FIG. 1B—top) indicates a steady increase in the number of scRNA-Seq biomonthly binned publications between 2014 and 2021 where data is available. Importantly, the number of single cells profiled in experiments is increasing from a general average of 100 cells beginning around 2015 to over 10,000 cells per study in 2021 (see FIG. 1B bottom). As these trends are only expected to continue increasing, it is anticipated that a plethora of additional transcriptomic information will become available, the integration of which into global, accessible datasets will further aid in the development of not only machine learning algorithms, but fruitful data reanalysis revealing novel biological insights. Performing additional study indexing on at least a monthly basis at minimum to allow for integration of recently published studies into model training cycles is anticipated, but it should be noted that ad hoc re-training can be conducted anytime using public or non-public datasets in less than 24 hours using existing computing infrastructures.

### Data Extraction.

Each indexed study was passed through an automated data loader customized to each unique input source (e.g. GEO) vs. AF) to automatically extract scRNA-Seq count matrices. All supplementary files associated with a particular study were first categorized, looking for delimited file type extensions used for either transcriptional data or metadata (e.g., .csv, .tsv, .h5, .h5ad, .mtx, etc . . . ). In cases where expression data is stored as multiple files (e.g. 10× Genomics matrix .mtx barcodes .tsv genes .tsv triplet format), pairs of common filenames were matched by stem using text-similarity unsupervised clustering. Metadata when present, including cell type annotations, is typically found in separated delimited files and is identified by matching filename substrings “meta OR metadata OR annot OR annotation”. Files identified as potential expression or metadata were then batch downloaded using the aria2 utility for further processing.

### Data Transformation.

For each datafile in a study, an attempt to load, parse, and standardize gene expression data, and then match it with any associated metadata was made (see FIG. 1C-top). In most cases, expression data is stored in a delimited file structure (e.g., .txt, .csv, .tsv formats) where each row and column correspond to cells and genes, respectively, or vice-versa. The major steps in file loading and standardization include first identifying file delimiters based on the most common present in the first line of the file (i.e. tab, space, comma, etc.). Second file dimensions are determined using a heuristic function that calculates the bytesize of the first N-lines of a given file and compares it to the total file size. Delimited files exceeding 100,000 projected rows or columns are read using a bespoke lightweight data parser, SRead, which distributes line reads across a unified thread pool for rapid data loading. Smaller files are read using the python pandas read table function using the identified delimiter. The final output is yielded as a pandas DataFrame object. Further, gene names are standardized to gene symbols using a comprehensive dictionary of gene IDs, synonyms, and symbols, where whether or not a row or column in the loaded DataFrame contains gene information is identified and set as the index or header, respectively. Depending on the initial data frame orientation, the orientation is corrected to follow tidy data conventions such that rows correspond to cells (observations) and columns correspond to genes (variables). Columns containing string-like characters are assumed to correspond to cell index names or associated cell-metadata, while those containing floating point or integer values are assumed to be expression data. An attempt is made to match rows or columns of metadata with standardized row indexes of a given sample file. If a high degree of concordance is found between a data matrix and files flagged as potential metadata, it is assumed that the file corresponds to cell-level metadata and both dataframes are aligned together for final integration. Finally, expression data is converted into compressed-sparse row (CSR) matrices map together with align metadata (if-any) using the annotated dataset (e.g. h5ad AnnData) library. These H5-like objects are then uploaded to a Google Cloud Storage (GCS) bucket as unprocessed, standardized data sets suitable for downstream processing.

### Data Preprocessing.

Before a scRNA-Seq dataset was utilized, it underwent additional preprocessing. The most commonly used packages for scRNA-Seq processing and analysis, scanpy (python) and seurat (R), were not originally designed for high-throughput batch processing of thousands of scRNA-Seq datasets. Many computational steps, including covariate regression, batch correction, nearest neighbor calculation, and dimensionality reduction, can take significant time for datasets exceeding 100,000 cells. To enable the models of the present disclosure, we developed scanpyRAPIDS, the first single cell analysis framework that enables complete end-to-end GPU-accelerated scRNA-Seq preprocessing. Leveraging the CuML, CuGraph, and CuPy python libraries from RAPIDS.AI [35], we reimplement the entire standard scRNA-Seq preprocessing pipeline from basic QC through batch correction, dimensionality reduction and clustering residing entirely in GPU memory (see FIG. 1C-bottom). Relative performance gains compared to traditional CPU-bound analysis is dependent on both the size of the input data and functional requirements of data preprocessing. For example, the disclosed scanpyRAPIDS implementation of the popular harmony batch correction algorithm successfully integrates 209,264 cells from 107 individual samples representing a time course of iPSC induction in 201.1 seconds on an NVIDIA Tesla T4 GPU, compared to 1,204.6 seconds on a 16-core vCPU instance with 100+GB RAM. This presents a 6-fold speedup in runtime that continues to scale linearly with dataset size.

Using scanpyRAPIDS, all raw H5AD objects from the previous stage were concurrently preprocessed, parallelized across four NVIDIA Tesla T4 16 GB GPUs. In brief, cells with less than 200 counts or genes expressed in less than three cells in a dataset were filtered out. Cells with greater than 20% mitochondrial read fraction were assumed to be dead or damaged cells, and filtered out. Cells whose total counts exceeded two times the standard deviation of log-normal total counts for all cells in the sample were assumed to be damaged outliers, and filtered out. Total counts were normalized to 10,000 reads per cell and subsequently log-normalized. For sample-level visualization, highly variable genes (HVG) were calculated, keeping genes with a log-normed mean between 0.0125 and 4, and a minimum dispersion of 0.25. HVGs were then z-score scaled to +/−10. The effect of cell read depth (total counts) on expression of each HVG was regressed using an CUDA-accelerated ElasticNet regressor. Lastly, principal component analysis (PCA) was performed, with the number of components determined based on the number of post-filtered cells present in the sample. Nearest neighbor calculation was performed with n_neighbors set to 30. Both 2D and 3D UMAP dimensionality reductions were run, with min_dist set to 0.3. Lastly, unsupervised leiden clustering was performed, with resolution determined, like PCA components, on the number of post-filtered cells in the sample. Post-processed H5AD AnnData objects were then uploaded to a GCS bucket. For cases where multiple samples were preprocessed for a given study, batch correction and re-clustering were performed using the described approach with the disclosed GPU-accelerated implementation of the Harmony algorithm [37].

### Data Storage.

When determining the optimal active data storage format, two concerns were addressed. Firstly, as total preprocessed data repository contains nearly 1 TB of data, which is expected to grow overtime and will need to be shared between team members, local on-disk storage would not be practical. Second, the need to rapidly load, inspect, and validate preprocessed data prior to final integration made traditional disk-mapped data formats such as HDF5 (and by extension, H5AD) limiting due to I/O throughput and cloud-access flexibility perspectives. As a result, a bespoke data storage model, SingleCellData (SCD), was designed built on top of the TileDB API. TileDB is a cloud-native data storage solution that integrates with cloud storage solutions such as GCS and S3, with explicit support for multidimensional, sparse array storage and parallel, chunked I/O operations [38]. The conversion of the preprocessed H5AD objects into SCD format allowed rapid access and the validation of the preprocessing quality for the datasets.

### Cell Type Annotation & Label Transfer.

A total of 1,712 unique studies with 10,000+ associated data files were successfully preprocessed and stored using the above methods. Approximately 20% of preprocessed data had cell type annotations available, requiring a two-step label transfer procedure to first project coarse cell types onto the unannotated data using annotated data as a reference, followed by manual verification and correction of labels to account for unknown, study-specific cell types.

### Coarse Label Transfer of Known Cell Types to Unannotated Data.

Projection of both annotated and unannotated cells into a common latent space was sought using a deep autoencoder model in order to cluster similar cell types together and transfer labels of nearby known cell types onto unannotated cells. To that end, a spherical variational autoencoder (sVAE) was trained with 30 latent dimensions on all preprocessed gene expression profiles (see FIG. 1E). In brief, sVAEs differ from conventional variational autoencoders (VAE) in the use of a non-normal prior distribution for parameter regularization. Early work applying sVAEs to scRNA-Seq data has shown benefits compared to traditional VAE in terms of embedding stability, leveraging the von-Mises-Fisher (vMF) spherical distribution [39]. For the disclosed sVAE, the PowerSpherical distribution was used because it offers improved numerical stability during model training [40]. Nearest neighbors were determined using cosine similarity relative to 30 embedded latent dimensions using CuML, followed by unsupervised leiden clustering with the resolution hyperparameter set to 2. For each of the 4,200 identified clusters, cell type classifications were averaged, taking the most common annotated cell type of a cluster to be that cluster's true label. Verification of each cluster annotation was performed by decoding the mean embedding vector of each cluster to obtain a denoised, average gene expression profile for that cluster, and examining the highest expressed genes for correlations between canonical marker genes and predicted class types [41,42]. The process was then repeated, re-grouping cells into high-level subtypes (i.e. B Cells, T Cells, Neuronal Cells, etc.) to obtain more refined subtype classifications.

### Quality Control and Manual Correction of Annotations.

For each annotated dataset, cell type assignments and clustering quality were compared to available figures published in corresponding studies. Cases where annotations were too broad, or did not match tissue-specific labels found in the study, were manually identified and corrected. A total of 899 out of 1,712 studies were verified to pass quality control, with an initial focus on the largest and most diverse datasets available. In total, just over 28,000,000 single cells were contained in the dataset reflecting 840 unique cell types, including 55 cancer subtypes and 156 distinct cell lines.

### Example 2 Model and Model Training Strategy

The model used in these Examples is illustrated in FIG. 1D. The model is a Deep Neural Network (DNN) with 281,397,066 trainable parameters that accepts normalized RNA expression input and outputs predicted cell type fractions (see FIG. 1D—left).

### Primary Data Input & Preprocessing.

The model accepts, by design, nearly all coding and non-coding human genes, for a total input size of 28,867 genes. This approach took advantage of the fact that DNNs, by nature of their overparameterization, do not suffer reduced performance from multicollinearity [26]; a phenomenon exhibited when one or more model input values (e.g. gene expression) are highly correlated that can negatively impact machine learning model performance. An overparameterized input space buffers performance against sparsity due to tissue heterogeneity and/or technical resolution exhibited by current transcriptomics platforms, allowing UCD to rely on alternate, non-canonical genes for cell type prediction in cases where canonical markers are not captured insufficiently sequenced.

Inputs were normalized on a per-sample basis rather than per-gene. It is presumed that model would be able to infer cell type signatures using relative differences in expression signals, and that per-sample normalization would make the model more robust to differences in feature scales between training and test data. Gene expression counts were first normalized to 10,000, followed by log₂ scaling so as to reduce the effect of heteroscedasticity on expression distribution. Each sample is z-scored to standardize variance across features. Lastly, min-max scaling is applied to rescale each feature value from 0 to 1, which is then used as input into UCD.

To further reduce reliance on canonical markers and limit the impact of sparsity, we introduced a two-step “corruption” process to our normalized sample inputs during model training. We first inject 5% Gaussian noise to the normalized expression profile of each gene (e.g. A normalized expression value of 0.8 from a given gene i will range anywhere from 0.75-0.85 following 5% gaussian noise injection). This is followed by a dropout layer, where 20% of input values are randomly set to zero. We reasoned that a combination of noise and dropout would further encourage the DNN to learn more complex representations of cell types that are robust to noise and missing genes.

Intermediate Layers. The core of the model disclosed in FIG. 1D consists of four fully connected dense layers of 8192, 4096, 2048, and 1024 neurons using an exponential linear unit (ELU) activation function. The size and depth of the network was determined through empirical evaluation of preliminary models of varying size on subsets of the final training dataset. Overparameterization, combined with dropout regularization, yielded superior deconvolution performance without evidence of overfitting. The choice of ELU over the more commonly used rectified linear unit (RELU) was made so as to avoid the potential for dying neurons during model training, as the gradient of a RELU activation is zero for weighted inputs less than 0 while ELU remains fully differentiable across all real numbers. After each dense layer, we applied 10% dropout for regularization, as it was seen empirically that larger values induced a noticeable drop in performance.

### Output & Post Processing.

The final layer of the model is a dense layer of 840 neurons, corresponding to all cell types available in our training database to-date, with a softmax activation function yielding cell type fraction estimates summing to 1. No additional regularization was applied to the output layer, for it was found to reduce overall performance.

The cell types in the resulting deconvolution sit at varying levels of cellular specificity hierarchies (e.g. ‘t cell’ vs. ‘cd4-positive, alpha-beta t cell’), a consequence of leveraging author-derived annotations and/or low-confidence in more specific labels. In order to account for prediction biases induced by this uncertainty (e.g. some t cells may in fact be cd4+t cells, while all cd4+t cells are themselves t cells), we employ a belief propagation (BP) step during output post processing. BP involves projecting initial cell type fraction estimates onto a cell type hierarchy subset from the Cell Ontology (see Supplementary FIG. 1D) [27], and summing probabilities upwards along the directed tree structure. In such a way, fractional probabilities assigned to certain cell type subclasses were captured to yield higher confidence estimates of deconvolution fractions for more generic cell types.

### Generation of Training Data.

### Parameterization.

The model described above in conjunction with FIG. 1D was trained using mixtures of simulated RNA-seq data (pseudobulk mixtures) generated from scRNA-Seq data. The process of generating a mixture is described in the following steps:
1. The total number of cells (T) comprising a mixture was selected. Given the goal to develop a model robust to both low-input (i.e. single cell ST) and high-input (i.e. bulk RNA) samples for deconvolution, a value from 1 to 10,000 was randomly selected with uniform probability.
2. The number of unique cell types (N) in a mixture was chosen. Anywhere from 1 to 32 cell types was selected to appear in a given mixture with uniform probability. The maximum value of 32 cell types (although parameterizable for future training) was assigned after analyzing the cellular diversity of all curated scRNA-Seq datasets, and taking the nearest log 2 value of the 95% percentile for the number of unique cell types per dataset. Selecting cell types with uniform probability has the effect of oversampling cells with low representation in the dataset, which improves model performance on rare classes.
3. The mixture fraction ratios F for N cell types were assigned. We assigned a random fraction ratio Fᵢ for each cell Nᵢ in a given mixture, such that all fraction ratios summed to 1.
4. Expression data for cell types are accumulated and averaged together. For each cell type Nᵢ in a sample, we randomly selected Nᵢ*T cells of that type from our uniformly preprocessed, integrated scRNA-Seq database. In cases where the required number of cells exceeds the total number of cells of a given type available in the dataset, the maximum number possible were added to the mixture, without duplication. Once all required cells were randomly selected, expression profiles were averaged together with a simple mean, resulting in a pseudobulk RNA expression profile with a known cell type fraction.

### Mixture Formation Via Rapid Data Integration.

The process of pseudobulk sample generation was implemented in python and optimized for high-performance execution using the python numba package [28]. All hyperparameters T, N, and F were precomputed as described above prior to generating mixtures, and cell type array row locations were pre-indexed to avoid repeat searches and improve performance. A total of 10 million pseudobulk mixtures were generated over the course of 18 hours at a rate of 150 mixtures per second using a total corpus of 28 million annotated single cells into a 28,000,000×28867 compressed-sparse-row (CSR) matrix, running on a Google Cloud Engine (GCE) n2d-standard-224 virtual machine (VM) instance with 224 vCPU cores and 896 GB system RAM. The choice of 10 million pseudobulk mixtures was made by training multiple iterations of UCD with stepwise increases in training dataset size, noting the impact the amount of mixture examples had on model performance (see FIG. 1E). An increasing logarithmic relationship was observed between training data size and performance, and determined 10M mixtures to be the optimal size for initial model evaluation as a tradeoff between model accuracy and training time. Increases in size offered diminishing projected returns with respect to theoretical peak performance (see FIG. 1G). Ultimately, these training parameters can all be customized as future training sets become more expansive beyond 840 cell types and/or if necessary for extended accuracy in use cases where runtime beyond 18 hours is not limiting given the rapid nature of the overall end-to-end training time.

### Model Training.

The model described above was implemented and trained using Tensorflow 2.5.0. The Adam optimizer was utilized for supervised backpropagation with a learning rate of 0.0001 and an effective batch size of 256. Pseudobulk training data generated as described previously was serialized into TFRecord objects and saved into a separate GCS bucket, subsequently fed into the model described above in conjunction with FIG. 1D using the tf.data API. The model described in FIG. 1D was trained across 50 epochs over the course of seven hours running a preemptible Google Cloud Engine (GCE) a2-megagpu-8g instance, comprising eight NVIDIA A100 40 GB GPUs, 96 CPUs, and 680 GB system RAM. A train-test-split ratio of 80/20 was selected for training validation, and test validation was conducted every five epochs and subsequently interpolated for visualization. Details of model training performance, as measured by mean squared error and Pearson correlation, are highlighted in FIG. 1E.

Example 3—Synthetic Mixture Generation. To assess the performance of the model trained in Example 2, pseudobulk mixtures of 10,000 PBMCs were generated from well characterized, baseline datasets (see Table 1).

TABLE 1

| Dataset Description | Source |
|---|---|
| 10K PBMC Healthy Donor | 10X Genomics |
| 5K PBMC Healthy Donor | 10X Genomics |

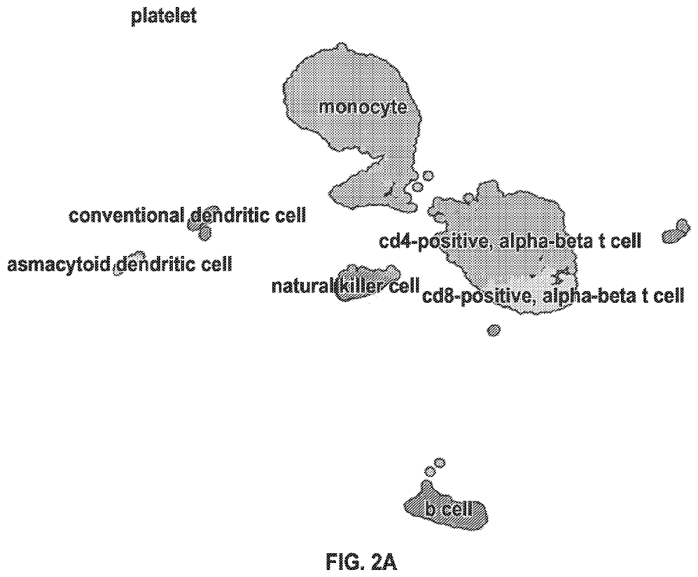

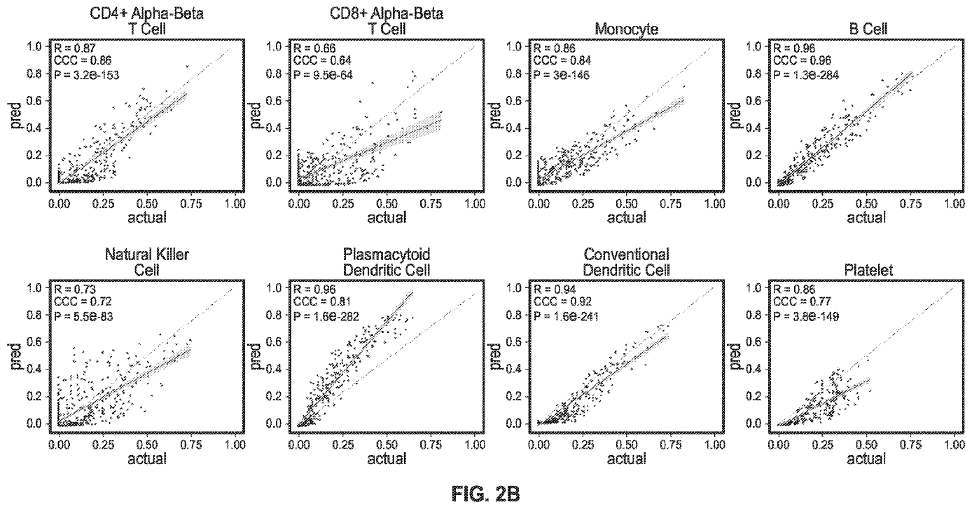

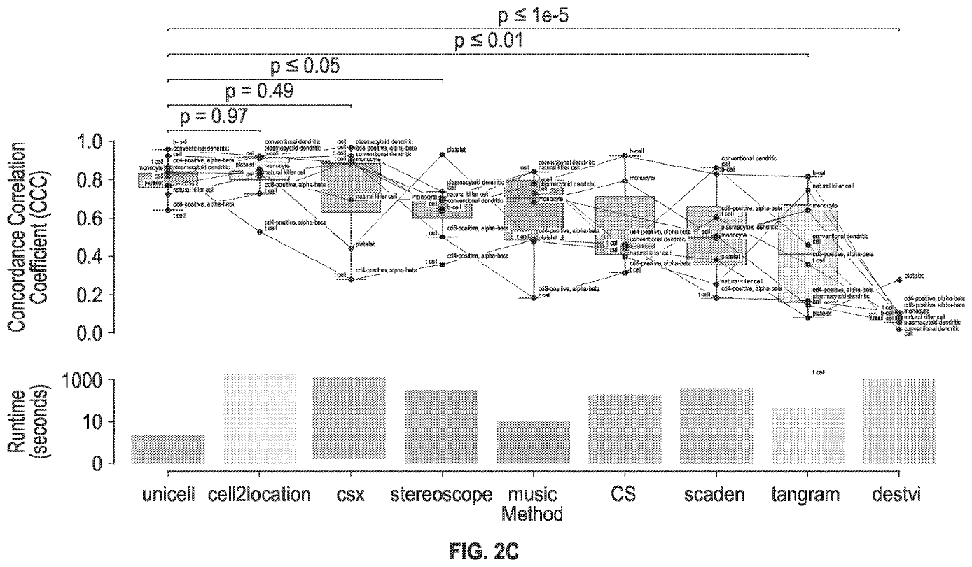

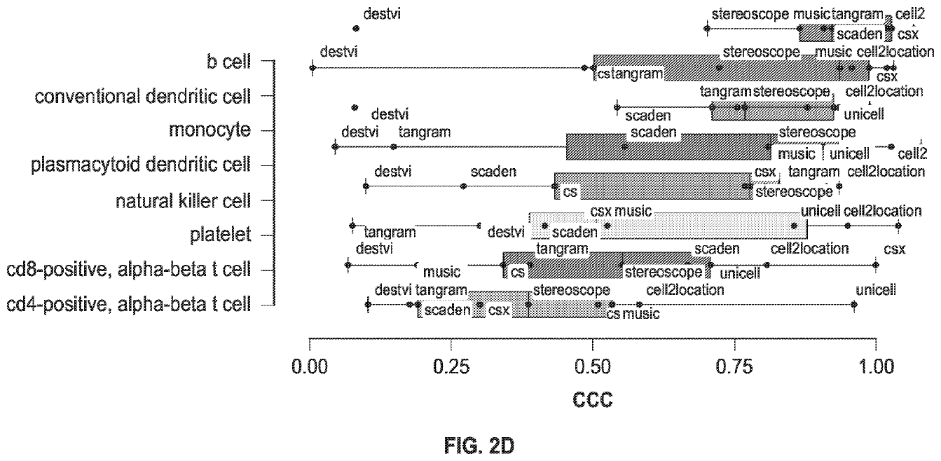

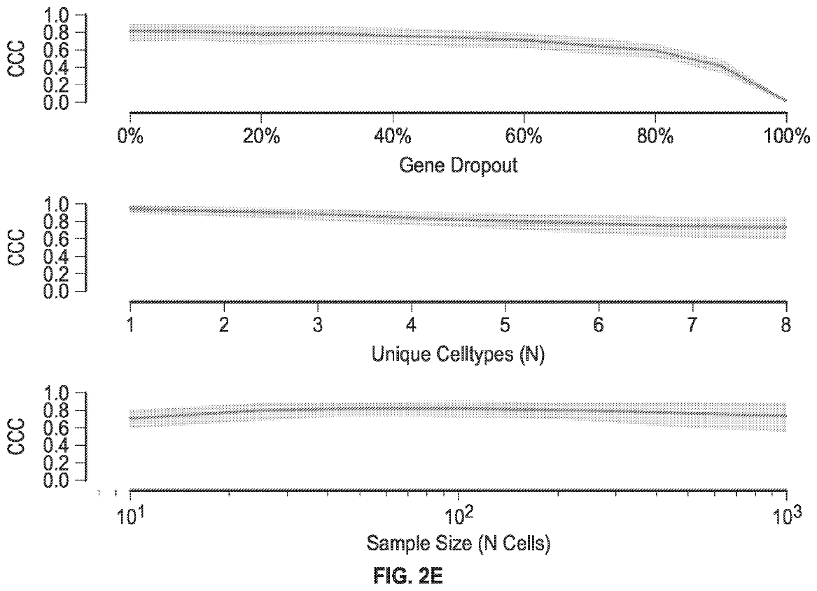

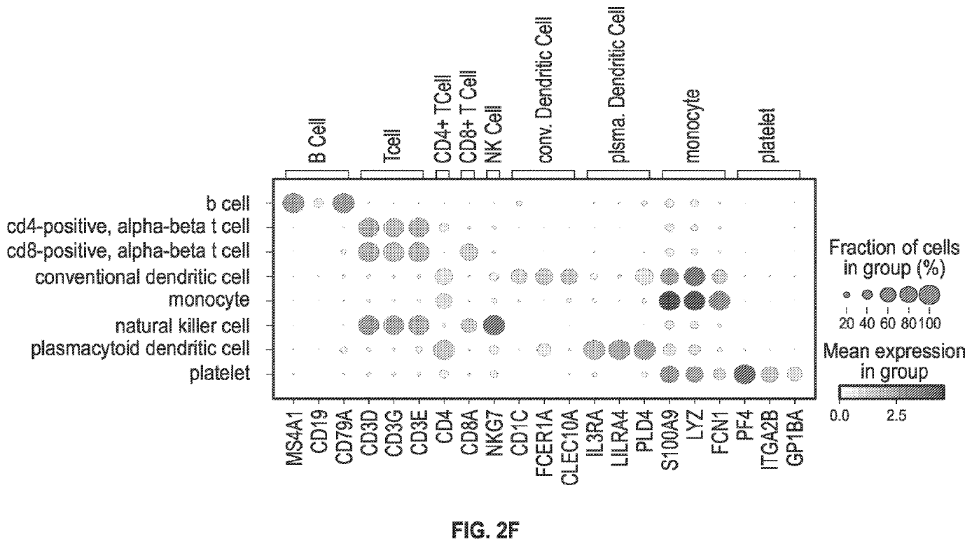

scRNA-Seq preprocessing, dimensionality reduction, and clustering, followed by manual cell type annotation using canonical markers, was performed, identifying eight unique cell types (see FIG. 2F). Using this dataset, the UCD generate_mixtures utility function was used to generate 500 pseudobulk mixtures of 100 cells, each containing five randomly selected cell types.

### Example 4—Performance Evaluation

Mixtures were deconvolved using eight competing approaches including CIBERSORT (CS), CIBERSORTx (CSx), Scaden, MuSiC, destVI, Tangram, Stereoscope, and Cell2Location. A tailored reference dataset was generated for all competing methods by preprocessing and annotating a secondary PBMC dataset containing 5,000 cells with matched profiles for all 8 cell types in our original mixture source dataset (see Table 1). Because existing deconvolution methods are sensitive to collinearity, input dimensionality was limited to the top 7,000 most highly variable genes in the source dataset, as determined by the scanpy function sc.tl.highly_variable_genes using the seurat_v3 method. Unlike other alternative methods, CS utilized the LM22 bulk-RNA immune cell reference provided by the authors. Performance was measured on the basis of how well the model described above in conjunction with FIG. 1D was able to predict cell type fractions relative to ground truth. Results were reported using Lin's concordance correlation coefficient (CCC), a measure similar to Pearson's R, but one that is sensitive to both slope and intercept in addition to variance, making it a suitable metric for comparing deconvolution performance.

### Sensitivity Analysis.

Deconvolution performance of the model described above in conjunction with FIG. 1D is expected to be sensitive to several hyperparameters pertaining to model complexity, notwithstanding the total cells in a bulk sample, the number of unique cell types present, and fraction of gene dropout. Using the disclosed PBMC reference dataset, baseline mixture hyperparameters consisting of 100 cells, 5 unique cell types per mixture, and 0% gene dropout were used as a starting point. These hyperparameters were systematically perturbed 500 new mixtures were generated, followed by deconvolution and performance evaluation. Total cells in a sample varied from 1 to 1000. The number of unique cell types in a sample varied from 1 to 8. Then, the effect of gene dropout was tested by randomly removing between 0 and 100% of all expressed genes in each mixture at the input stage.

### Integrated Gradients Analysis.

Deep neural network (DNN) models are often described as being “black-box” in nature, whereby the underlying mechanisms correlating inputs to outputs are largely unknown. The ability to interpret DNN models is highly desirable in biomedical science, as it enables researchers to verify a model is learning to generate predictions using plausible mechanistic correlations. Furthermore, interpretability can potentially deliver novel insights into biological processes as they pertain to input genes correlating with model outputs such as cell types. Several approaches for DNN interpretability have been proposed, including model agnostic approaches such as Shapley (SHAP) values [43], Local Interpretable Model-agnostic Explanations (LIME) [44], and DNN-specific methods such as Integrated Gradients (IG) [45]. IG differentiates itself from competing approaches with respect to its scalability to large input dimensions, making it particularly appropriate for interpreting the disclosed model predictions with a 28,867 gene input space. While IG is only applicable to fully differentiable models, making it unsuitable for interpretation of ML methods such as gradient boosted trees or random forest, the disclosed model's implementation (described above in conjunction with FIG. 1D) as a pure DNN makes it fully compatible with integrated gradients. The goal behind IG is calculation of the effect a change in a particular input “i” has on a given output class probability “j”, expressed as the gradient (i.e. partial derivative of “j” with respect to “i”). The integrated component refers to the accumulation (i.e. mathematical integration) of local gradients for input “i” across an interpolated range of values starting from a zero-baseline to its true value within a particular sample. Integrated gradients for each input gene are then multiplied by a scaling factor representing the absolute difference between the baseline case and normalized sample expression level, such that only genes actually expressed in the sample being analyzed will yield non-zero input attributions. Intuitively, this enables one to attribute the importance of input (gene) “i” with respect to how much it is adding to (positive attribution) or subtracting from (negative attribution) the models overall output probability for a given class (celltype) “j”. The intuition behind this approach is visualized in FIG. 1F.

For IG Analysis (IGA) in the model described above in conjunction with FIG. 1D, the baseline interpolation function consisted of a 50-step linear interpolation of gene expression between zero and true sample values, multiplied by randomized gene dropouts (with a 50-step descending probability of 100% to 0% dropout, as a means of roughly simulating the effect of lower-read depth on absolute gene transcript detection). The integral of interpolated local gradients is approximated using a trapezoidal Riemann summation.

### Example 5—Benchmarking Dataset Acquisition & Preprocessing Secondary Spatial & Bulk-RNA-Seq Data

Five publicly available, temporal spatial transcriptomics datasets from a mouse bilateral renal IRI model developed by Dixon et. al 2022 [46] were collected. Breast Invasive Adenocarcinoma and Prostate Adenocarcinoma Spatial FFPE samples were downloaded from the 10× Genomics Datasets repository (see Table 1). Colorectal ST data was downloaded from the 10× Genomics Datasets repository by means of the scanpy function sc.datasets.visium_sge.

Bulk-RNA Seq lung data originating from 5 mg tissue samples of patients with ALI, IPF, and healthy lungs collected by Sivakumar et al. 2019 [47] was downloaded from the Gene Expression Omnibus (GEO) using accession GSE134692. Bulk-RNA Seq data of white matter lesions sampled from patients with multiple sclerosis or healthy controls by Elkjaer et al. 2019 [48] was downloaded from GEO using accession GSE138614. Bulk-RNA Seq data from Fadista et. al 2014 [49] comprising pancreatic islet samples from individuals with varying states of T2DM was downloaded from GEO using accession GSE50244. Severity of T2D is monitored long-term by the measure of Hemoglobin % A1c (HgA1c). Values less than 5.7% were considered “Normal”, values between 5.7 and 6.4 were considered “Prediabetes” while values higher than 6.4% indicate a patient has T2DM [50]. Samples were stratified by patient HgA1c clinical thresholds into three groups: normal, prediabetes, and diabetes.

For each bulk-RNA-Seq dataset, TMM-normalized (Lung & Pancreas) or raw (MS) count data, gene annotations, and clinical metadata were integrated into a single annotated dataset object. No filtering was performed on genes or read counts, however read depths for raw counts were normalized to 10,000 per sample. Depth-normalized count data was then passed to the model described above in conjunction with FIG. 1D for deconvolution. Wilcoxon rank-sums test was used to determine differences in deconvolve cell type fractions between groups, with Bonferroni correction for multiple testing.

### Example 6—Primary Non-Small Cell Lung Cancer Data

Paired biopsies reflecting tumor and matched adjacent normal tissue were obtained from a patient with non-small cell lung cancer (NSCLC) undergoing surgical resection at the Mount Sinai Hospital (MSH) via the Mount Sinai Pathology Core. Samples were dissociated into single-cell suspensions using the Miltenyi Tumor Dissociation Kit (130-095-929) and the Miltenyi gentleMACS Dissociator (130-093-235). Single cell suspensions were processed with the 10× Genomics Chromium Next GEM Single Cell 3′ v3.1 kit (PN-1000121), targeting 10,000 loaded cells per sample. Whole-transcriptome sample libraries were sequenced on a NovaSeq 6000, targeting 50,000 reads per cell. Sequenced data was processed through CellRanger, yielding filtered count matrices for use as input into downstream single-cell data analysis using the python scanpy package. Both count matrices were concatenated into a single merged dataset. Briefly, cells with less than 2000 or greater than 100,000 reads were filtered out, as well as cells that contained less than 200 or greater than 30,000 unique genes. Cells with more than 10% mitochondrial gene fractions were assumed to be dead or damaged, and excluded from further analysis. Cell counts were normalized to 10,000 counts per cell, and subsequently, the effects of total counts, percent mitochondrial counts, and cell cycle score were regressed out. Regressed, normalized counts were then log-scaled and z-scored with a min-max of +/−10. Highly variable genes were identified on the basis of a dispersion score of 0.1 or greater for genes with log-normalized expression values between 0.1 and 20. HVGs were used to generate 75 principal components. At this stage, we performed batch correction using harmony, which outputs a corrected principal components array for use in all subsequent analysis steps. Calculation of nearest neighbors using our adjusted PCA vectors was done with n_neighbors set to 30. UMAP was used for final dimensionality reduction with minimum_distance set to 0.3. Leiden clustering was then performed to identify transcriptionally-related clusters, with resolution set to 1. Log-normalized counts were used as input into the model described above in conjunction with FIG. 1D to generate cell type prediction scores.

### Example 7—Model Benchmarking Using Single-Cell RNA-Seq Mixtures

For each of the eight cell types identified in our peripheral blood mononuclear cell (PBMC) reference dataset (see FIG. 2A), actual and predicted cell type fractions were compared across 500 simulated mixtures (see FIG. 2B). The pre-trained model (described above in conjunction with FIG. 1D) obtained a strong 0.816 average concordance correlation coefficient (CCC) across all cell types. The disclosed model performed comparably with current State of the Art methods such as Cell2Location (C2L) (p=0.97, see FIG. 2C top), despite the fact that C2L and competing algorithms were trained to exclusively consider the deconvolution of PBMCs. UCD offer 2-3 orders of magnitude improvement in runtime given its pre-trained nature: UCD returned results in 2.3 seconds, compared to C2L which required 31.1 minutes to converge to a solution using our benchmarking dataset and reference (see FIG. 2C bottom).

The disclosed model (described above in conjunction with FIG. 1D) was robust to changes in mixture hyperparameters (see FIG. 2E), with a minimal linear decrease in mean performance as sample complexity (i.e. number of unique cell types) increased. Model performance was found to slightly increase with more cells in each mixture sample. This reflects a reduction in signal-to-noise as multiple expression profiles were averaged together. When perturbing gene dropout, significant performance reductions were seen only after >80% of expressed genes in the benchmarking mixture samples were removed as inputs. This robustness to dropout suggests that the disclosed model (the model described above in conjunction with FIG. 1D) leverages nonlinear combinations of gene sets as the basis of cell type fraction predictions, and is resilient to the noise seen in transcriptomic data, especially at lower read depth.

### Example 8-Characterization of Pathophysiologie Cell Type Aberrations in Ischemic Kidney Injury

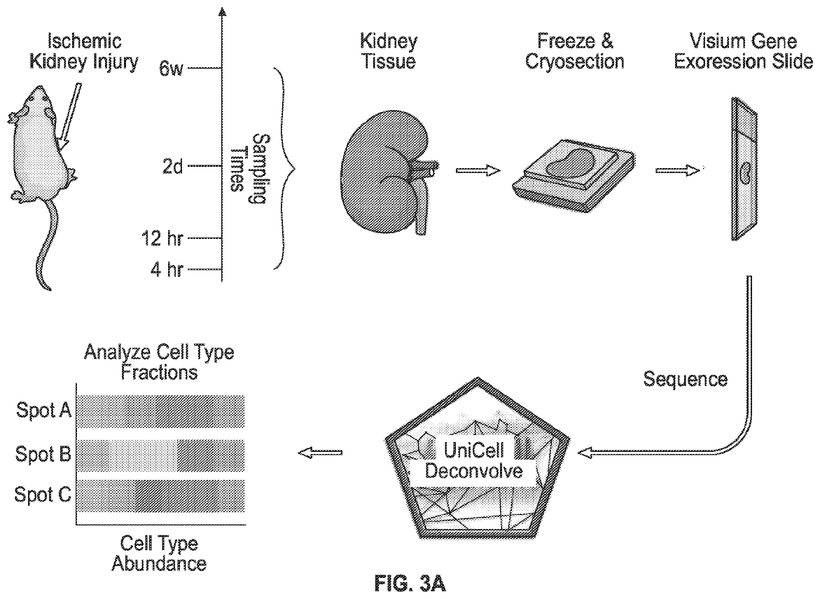

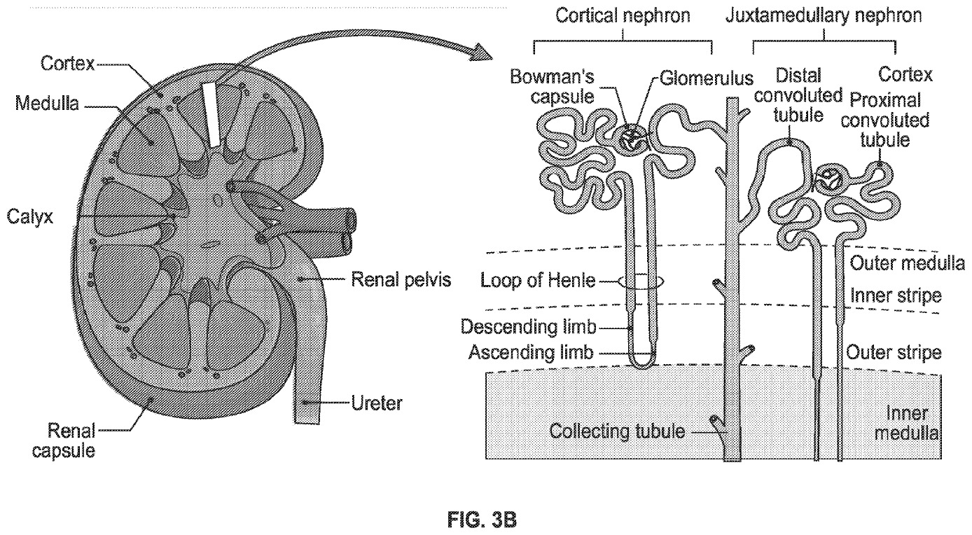

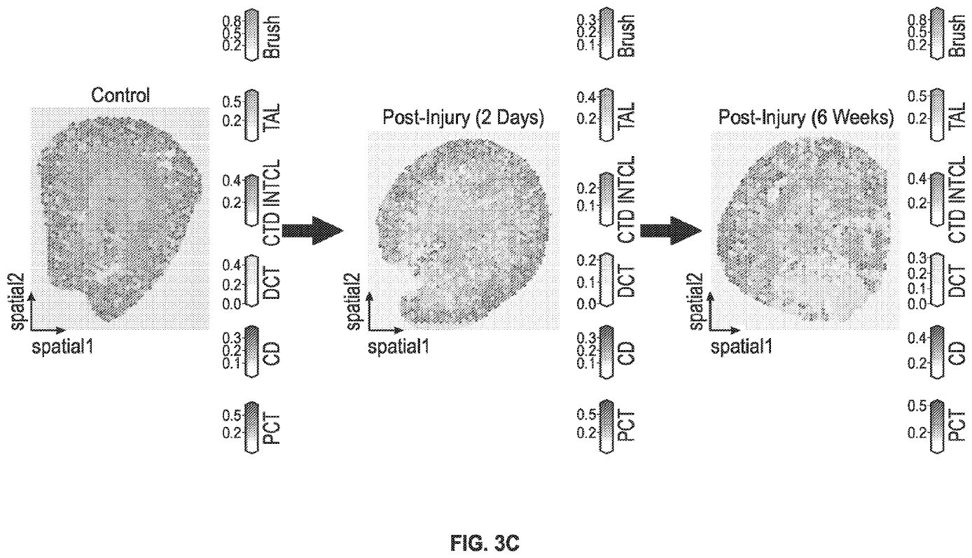

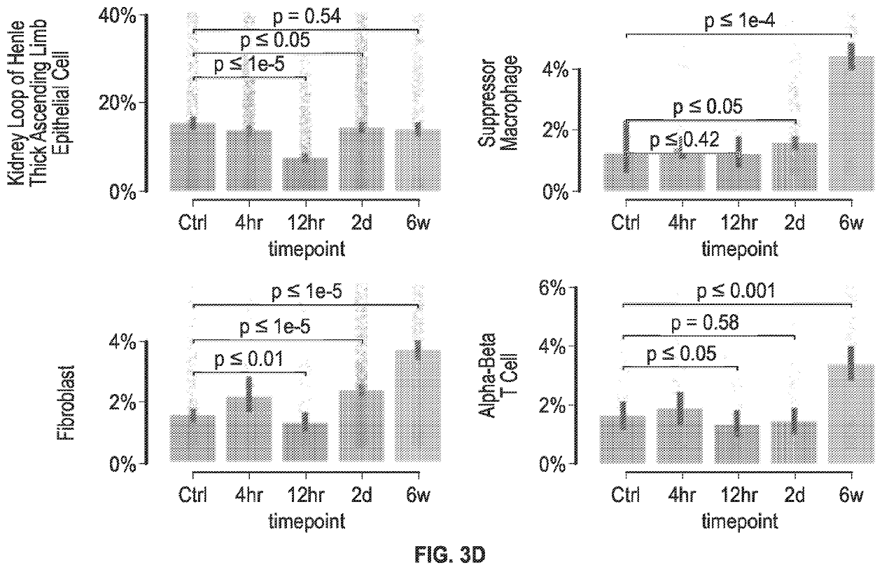

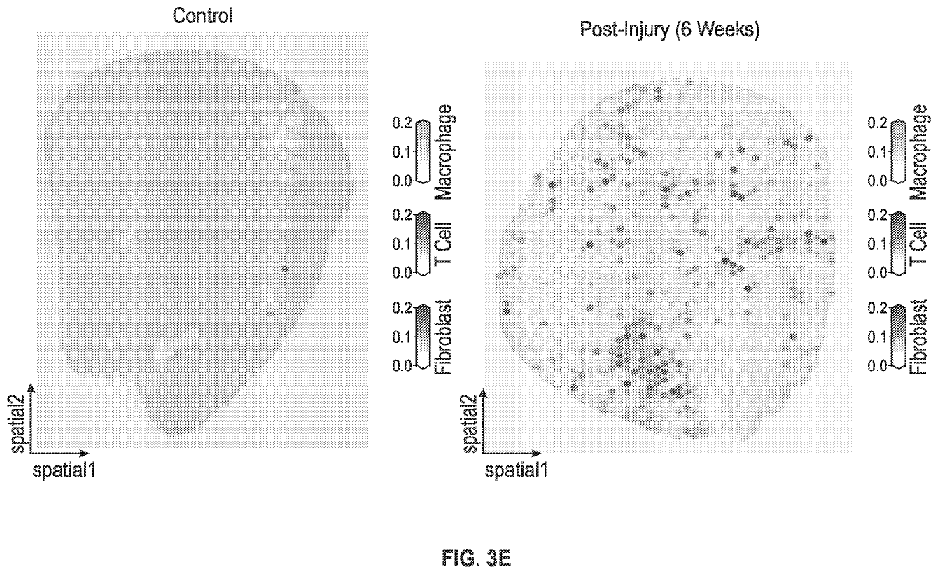

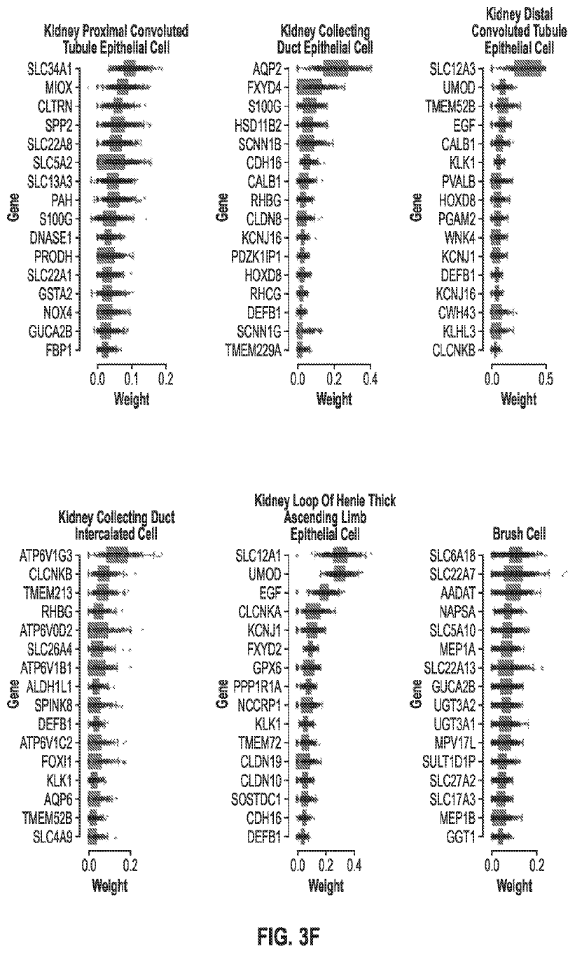

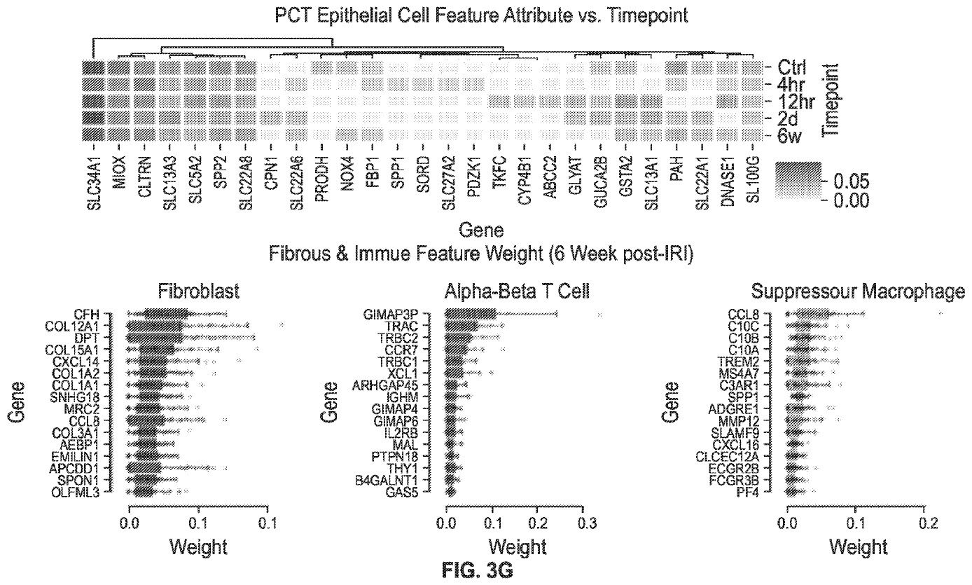

Kidney ischemia reperfusion injury (IRI) describes the oxidative stress and inflammatory damage induced by revascularization following a loss of blood flow and oxygen to cells of the renal system [51]. IRI is a common perioperative complication occurring during major trauma, shock, sepsis, or transplant, and understanding the pathophysiologic changes it induces is critical in developing strategies to mitigate its long term impacts [52]. Using temporal spatial transcriptomics data of coronal kidney tissue sections collected from a mouse bilateral renal IRI model, developed by Dixon et. al 2022 [46], the model described above in conjunction with FIG. 1D was leveraged to explore changes in kidney cell fractions associated with progressive IRI damage (see FIG. 3A). Deconvolution results were examined in the context of normal control tissue in FIG. 3C, comparing it with expected cellular organization as summarized in FIG. 3B. The disclosed model (described above in conjunction with FIG. 1D) identified spatial distributions of proximal (PCT) and distal convoluted tubule epithelial cells localizing correctly to the outer cortex zone of the kidney. The thick-ascending limb of the loop of henle (TAL/LOH) was localized to the inner-renal medulla, while cells of the collecting duct (CD) were identified to be distributed across the renal cortex with increased abundance in the medulla, as they coalesce into the renal calyx. Intercalated cells (IC) were identified mainly along the boundary zone of the outer medulla, consistent with IC preferential localization in the earlier sections of the CD [53]. The disclosed model also predicted “brush cells” in the outer medullary zone, which may correspond to the S3 straight segment of the PCT based on identified gene attributes (see Table 2 of Example 13).

This is unsurprising, as the morphology of PCT cells is that of brush-border ike, and the S3 segment displays the least degree of functional differentiation [54]. Specific genes UCD associated with all renal cell types were contrasted with established literature and are detailed in Table 2 of Example 13.

Next, changes in absolute cell type fractions predicted to occur following IRI were examined. The overall composition and spatial organization of major kidney cell types remained unchanged (see FIG. 3C—center & right). Increases in t cell, suppressor macrophage, and fibroblast content became apparent as early as two days post-IRI compared with control, peaking at the six week timepoint (see FIGS. 3D & E).

A notable gene attributed to t cells was CCR7. It has been shown that CCR7+t cells mediate kidney injury during transplant allograft rejection, suggesting a similar role in IRI [55]. Suppressor M2-like macrophages promote kidney repair after acute IRI by modulating innate immunity [52].

Fibroblast infiltrate at the 6 week timepoint (see FIG. 3G) was associated with complement factor-H (CFH) expression. The authors of the original study explicitly noted the inability to establish a link between CFH and fibroblasts from Visium data alone, and verified its selective expression among kidney fibroblasts using an independent single-nucleus RNA-Seq dataset [46].

While the canonical PCT marker SLC34A1 remains a consistent attribute of PCT cells across time points (see FIG. 3G), evidence of secondary markers overexpressed following injury is seen in the data, suggesting temporal physiologic changes to PCT cell function. The metabolic waste efflux pump ABBC2 has been shown to be overexpressed after acute renal IRI in mice [56], and exhibits increased attribution for PCT cells at 12 hours post-injury, suggesting overexpression and increased PCT stress [56].

Together, this data shows that the disclosed model (described above in conjunction with FIG. 1D) allows for the rapid provision of a comprehensive picture of physiological changes underpinning the kidneys' response to IRI. Through cell type deconvolution in addition to feature attribute analysis, the disclosed model identifies physiologically relevant marker genes underpinning the transition from homeostatic renal function to chronic inflammation and fibrosis, while simultaneously capturing the complex interplay between fibroblasts, t cells, and immunosuppressive macrophages.

### Example 9—Deconvolving Tumor Biology

### Robust Malignant Subtype Identification & Cancer Feature Attribute Analysis.

Dysregulation of gene expression programs is a hallmark of cancer [57], and thus an effort was made to see if deconvolution of nonmalignant cells from cancerous cells using transcriptional profiles was possible. The disclosed model's (described above in conjunction with FIG. 1D) cancer detection and subtype classification performance was thus evaluated.

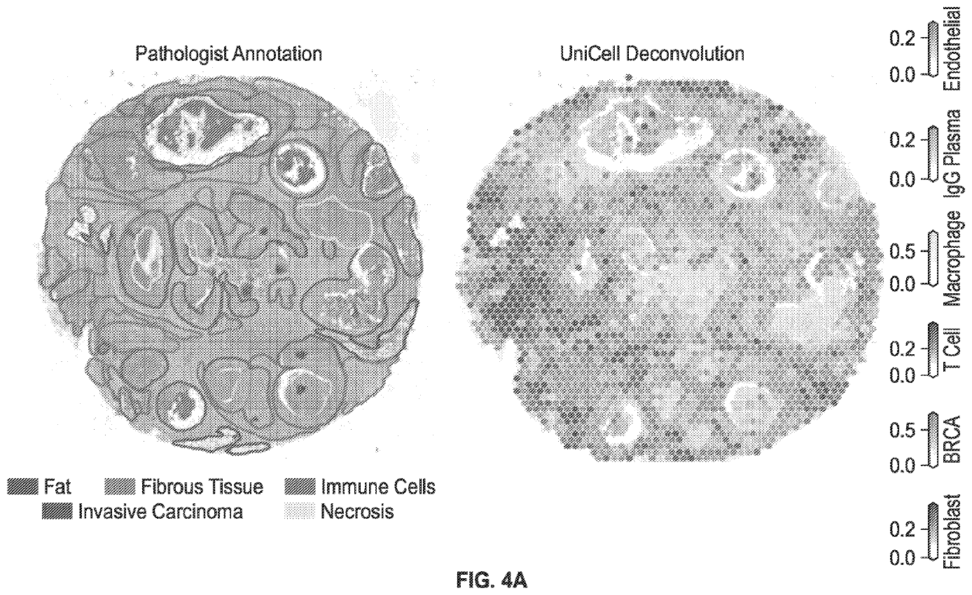

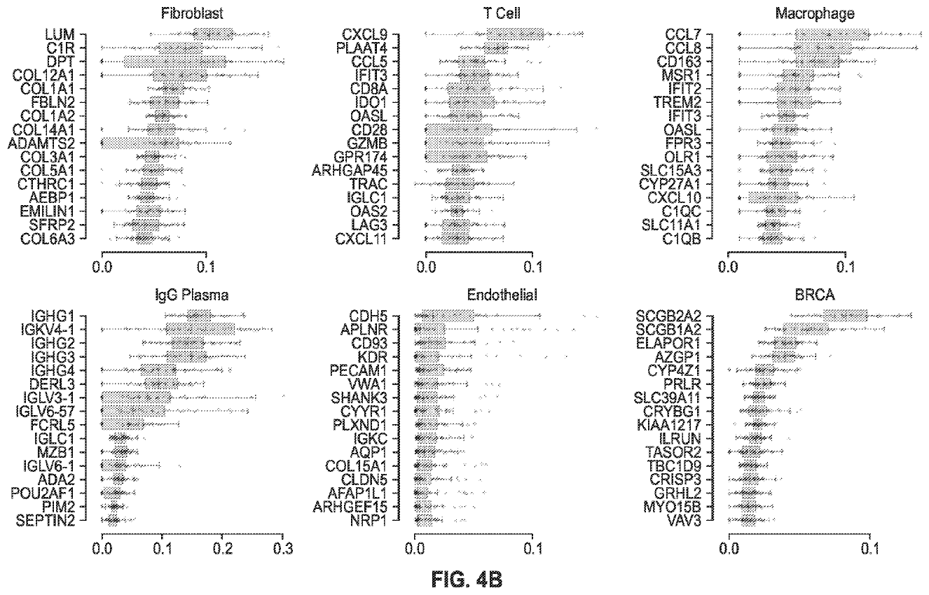

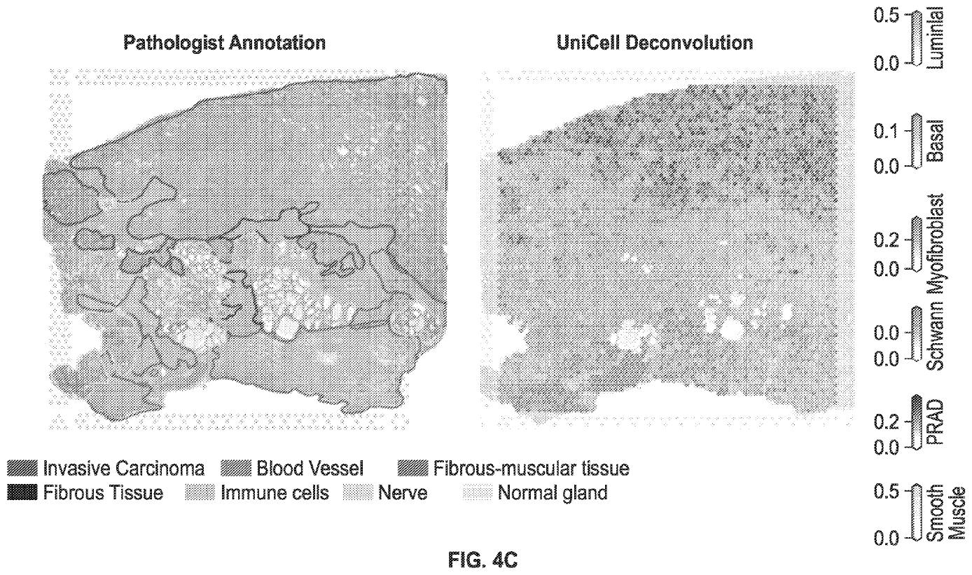

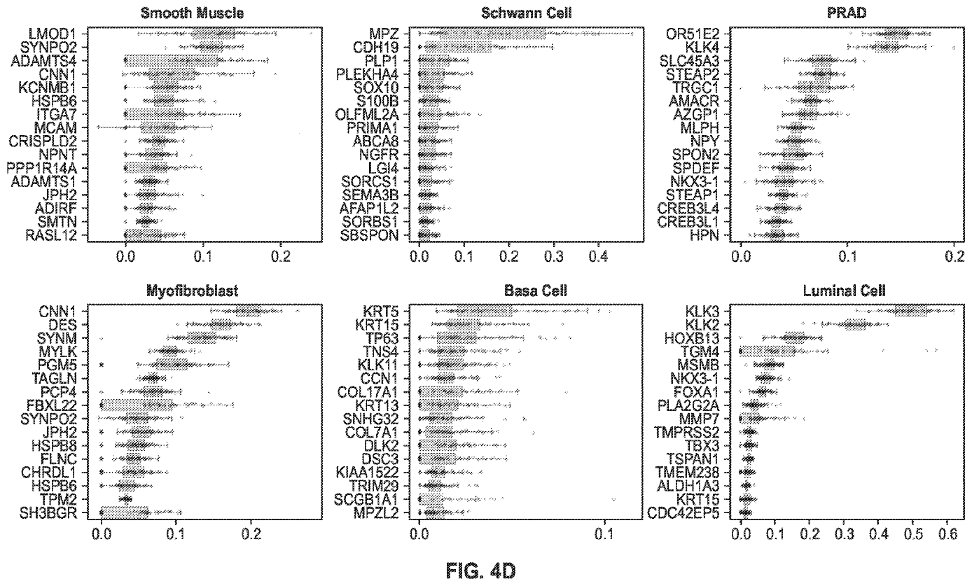

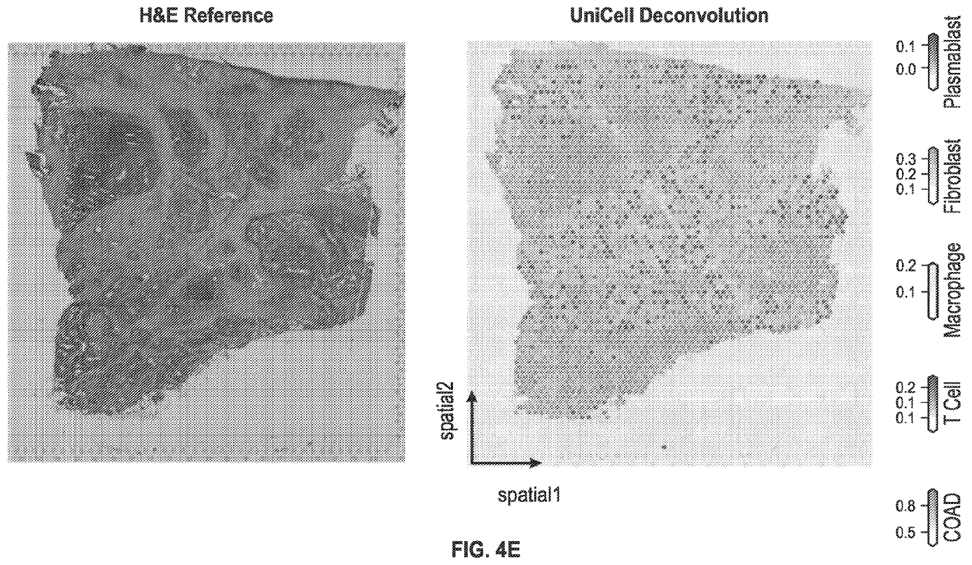

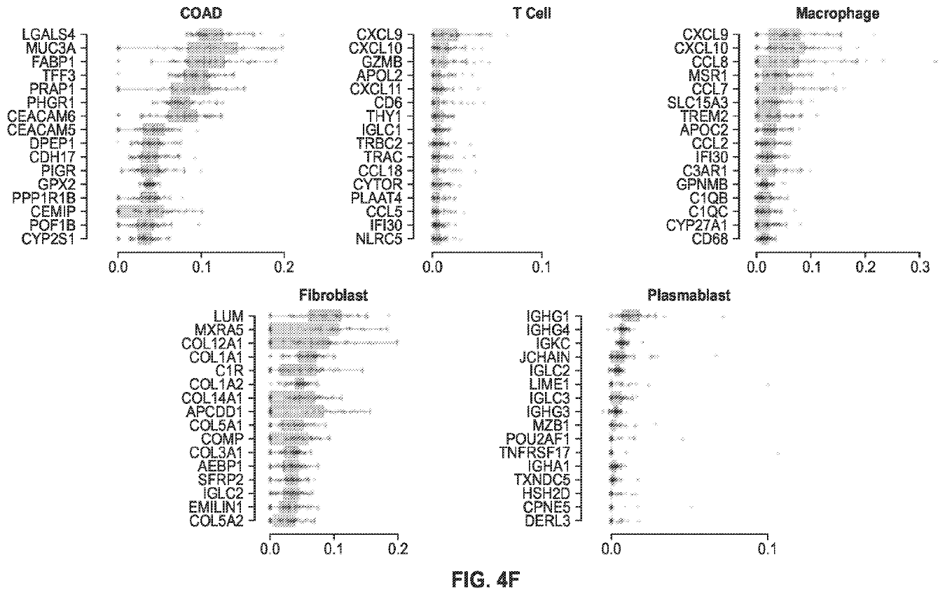

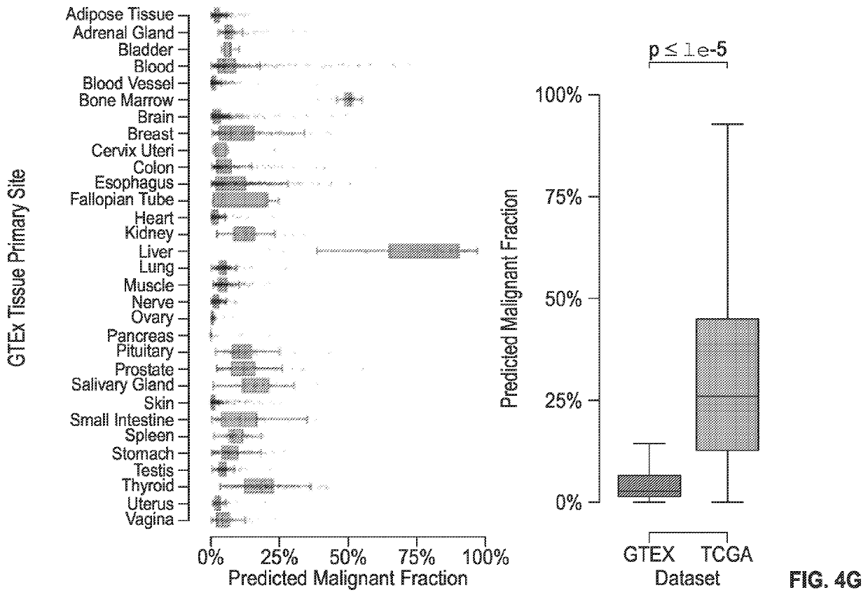

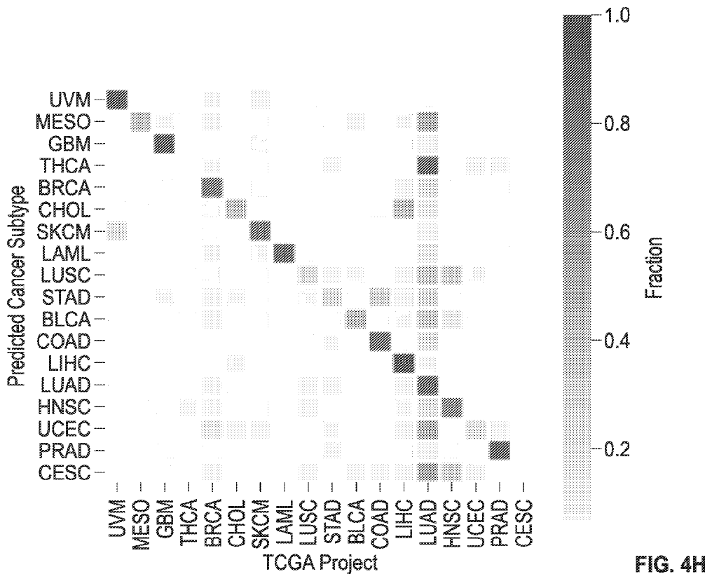

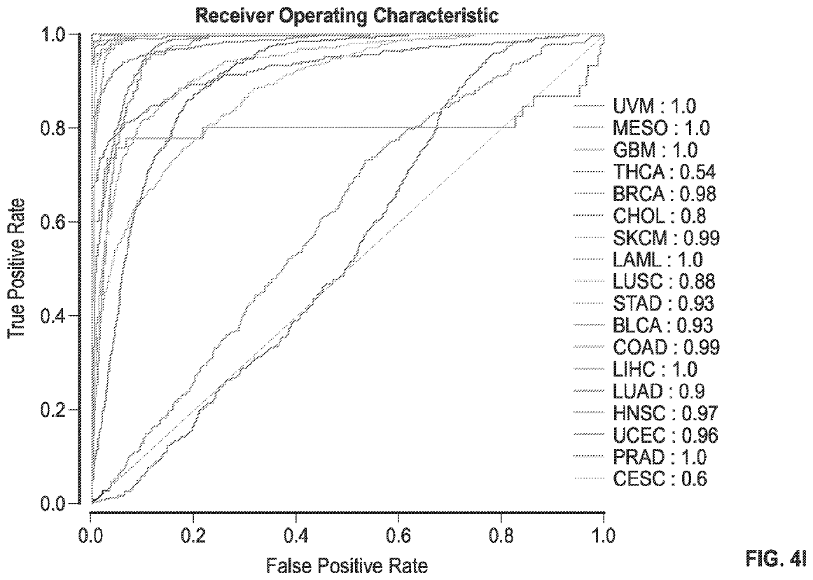

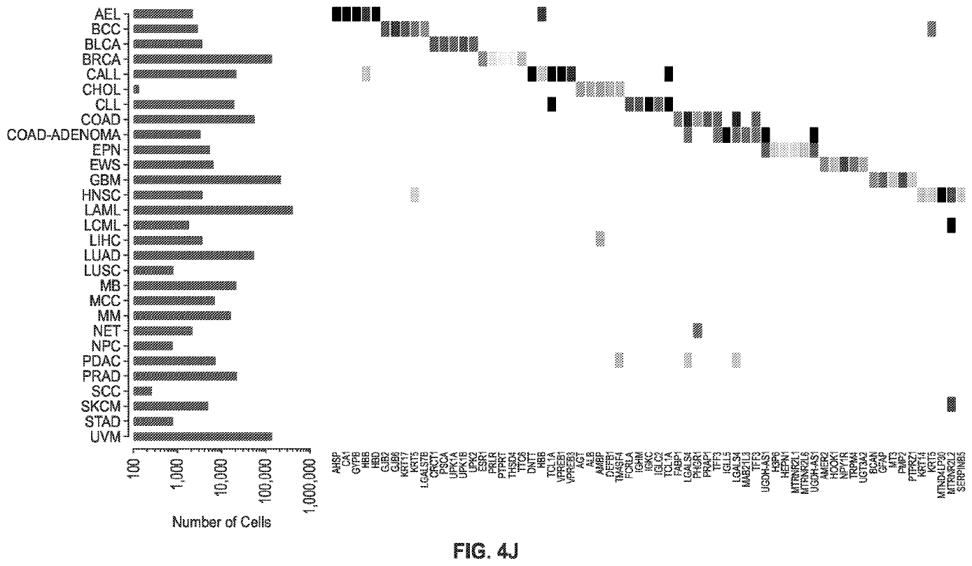

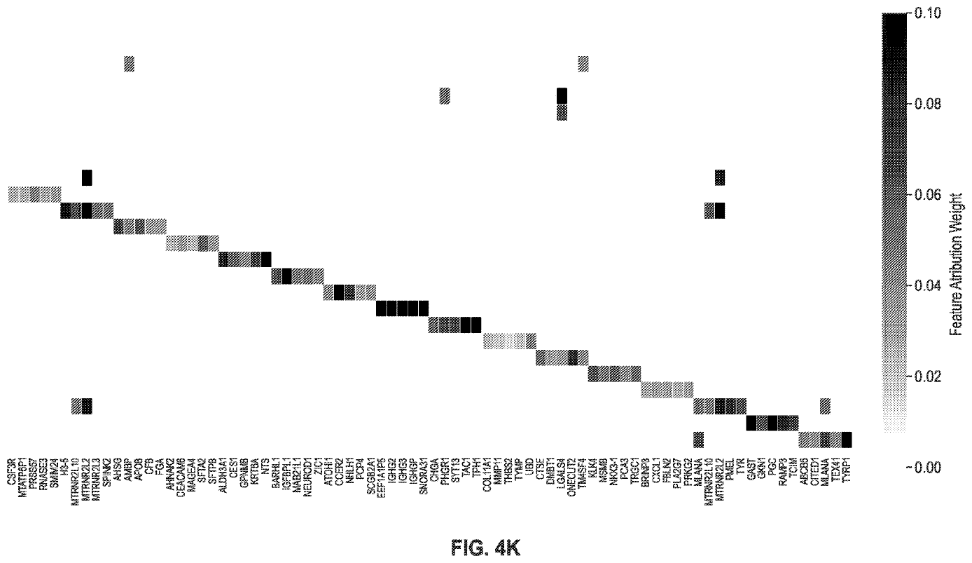

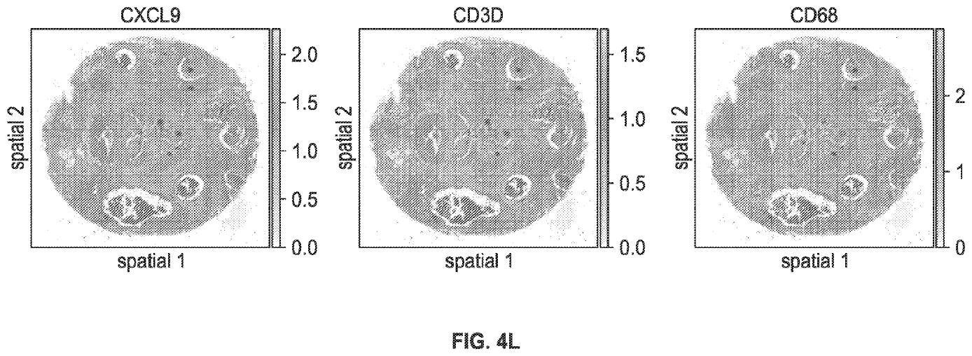

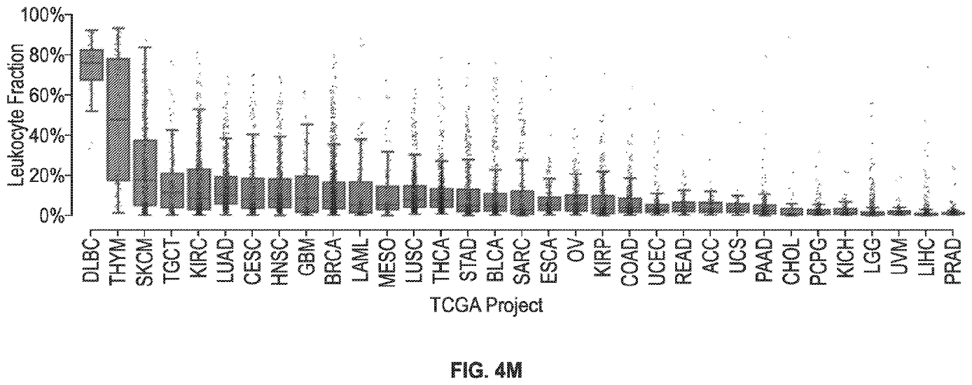

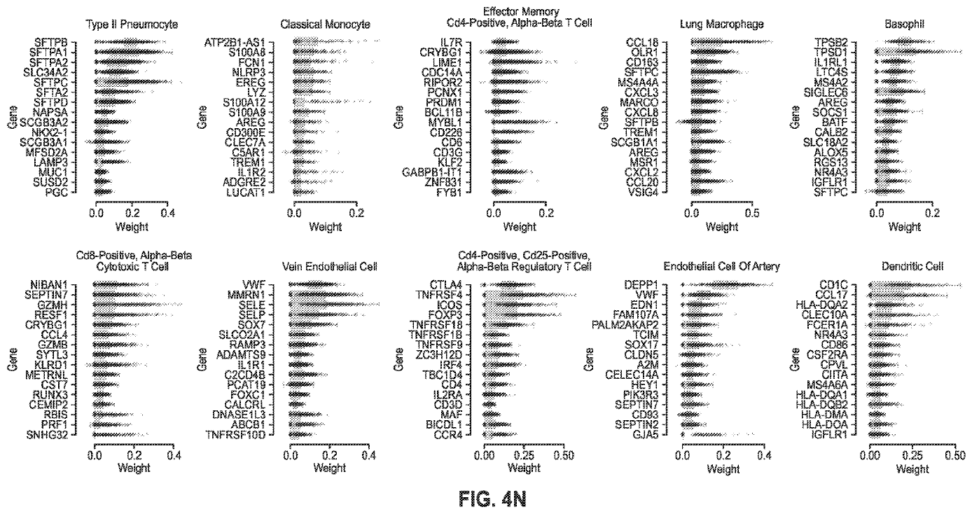

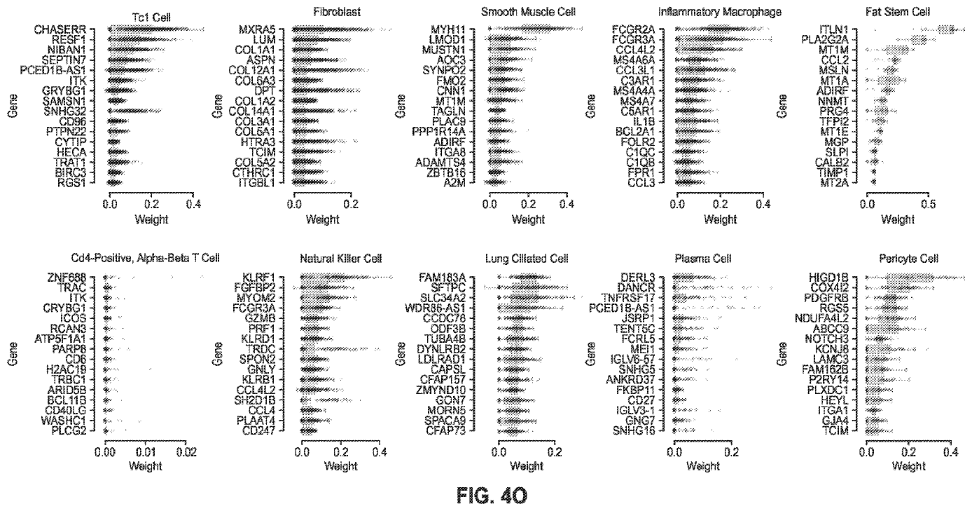

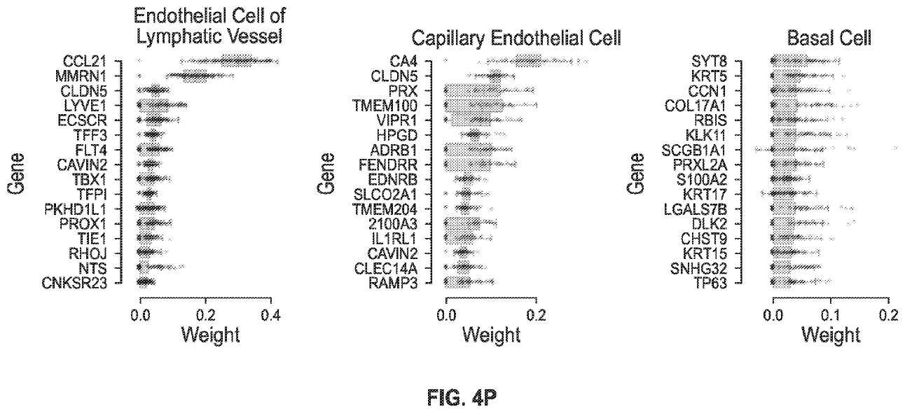

Testing the disclosed model's sensitivity to malignant versus normal tissues, bulk RNA samples were deconvolved from GTEx (n=7,845) and TCGA (n=10,459), predicting samples to be 97.3% vs 74% non-malignant (p<1E-5) when comparing median values of GTEx and TCGA samples, respectively (see FIG. 4G right). A notable outlier prediction is seen among GTEx liver samples (see FIG. 4G left), which can be attributed to sample-specific pathological, preprocessing, or quality control factors (or model training data label misannotation between non-malignant hepatocytes and liver hepatocellular carcinoma (LIHC)). Using deconvolved TCGA data spanning 18 cancer subtypes matched between model and TCGA, malignant cell results were re-normalized independently of non-malignant cell types to predict cancer subtypes (see FIG. 4H). The disclosed model achieved a micro-average area under the curve (AUC) of 0.889 across all cancers (see FIG. 4I), indicating strong classification capability.

To gain insight into the gene expression profiles learned by the disclosed model (described above in conjunction with FIG. 1D), the top-5 gene integrated gradient weights for all 1,143,791 primary cancer cells in the training database averaged by subtype were examined (see FIGS. 4J and 4K). Examining the results, it is seen that the disclosed model successfully learns gene expression profiles representing unique transcriptome signatures of subtype-specific malignancies. Demonstratively, prostate cancer adenocarcinoma (PRAD) is identified via NKX3-1, a distinct marker of prostatic cancers [58], as well as other genes such as PCA3, and FOLH1. For melanoma (SKCM), the disclosed model associates it with the expression of MLANA, the melanoma diagnostic antigen melanin-A [59], as well as genes such as TRYP1 and MTRNR2L2. Further inspection of the abovementioned gene features and others (see Table 2 of Example 13) demonstrates the disclosed model learned subtype-specific gene representations that appear to corroborate their relevance as suggested in prior studies.

### Example 10-Spatial Transcriptomic Deconvolution of Tumor Microenvironment

A diverse set of publicly available solid tumor spatial transcriptomic tissues, including Breast Adenocarcinoma (BRCA), Prostate Adenocarcinoma (PRAD), and Colorectal Adenocarcinoma (COAD) were deconvolved using the disclosed model (described above in conjunction with FIG. 1D). Where available, the disclosed model deconvolution results were compared to histological annotations performed by certified human pathologists to determine relative accuracy of underlying cell type predictions. Feature attribute analysis was performed for all predicted cell types, with pathophysiologic significance elaborated for each gene in Table 2 of Example 13 where appropriate.

### Breast Adenocarcinoma.

The disclosed model (described above in conjunction with FIG. 1D) correctly identified the most likely tumor subtype, BRCA, localized across ductal glands consistent with pathologists annotations (see FIG. 4A). There was strong concordance with pathologist-designated fibrous tissue deposits and fibroblast predictions, attributed to numerous well-established extracellular-matrix (ECM) genes including COL12A1, a gene previously implicated in pro-inflammatory stromal desmoplasia and tumor progression in several cancers [60]. Endothelial cells were detected throughout the tumor stroma, and particularly showed strong attribution to apelin receptor (APLN), a gene involved in maintaining pro-angiogenic states among endothelial cells, possibly indicating active tumor neovascularization [73].

The disclosed model identified multiple immune subtypes, including plasma cells, macrophages, and t cells, localizing to regions of pathologist-annotated immune infiltrate. Tumor-associated macrophages (TAMs) were found at or around areas of comedo-like tumor necrosis [61]. T cells were found to be localizing selectively around a distinct malignant duct located center-left of the tissue section, with attributed genes such as immune checkpoint costimulatory receptor CD28, as well as IFIT3, CCL5, and PLAAT4 implicating an active anti-tumor immune response. CD28 is required for an interferon-mediated immune response, coinciding with expression of interferon induced response protein IFIT3 [62]. The potent lymphocyte attractor ligand CCL5 is reported to be prospectively upregulated in tumor-infiltrating CD4+t cells following an initial immune stimulation to maintain t cell infiltration [63]. Furthermore, phospholipase A/acetyltransferase 4 (PLAAT4) has been identified as loosely expressed in t cells to support the adaptive immune response [64].

The disclosed model strongly implicates CXCL9 in the prediction of t cells, which is traditionally believed to be secreted by tumor cells themselves or TAMs to drive t cell recruitment [65]. When overlaying gene expression of CXCL9, CD3D (t cells) and CD68 (macrophages) (see FIG. 4-L), moderate spatial correlation with CXCL9 and CD3D (r=0.4, p=3E-29) is seen along the tumor-stromal interface, and weaker correlation with CD68 (r=0.17, p=1E-10). It is possible that cell-free RNA originating from apoptotic tumor cells in proximity to tumor infiltrating t cells may be captured during single cell encapsulation for sequencing. As the t cell category of UniCell's training data is a generalized category encompassing 191,425 cells of varying possible subtypes and originations, some of which may be tumor-associated, this may be reflected in results when analyzing cancer datasets. Nevertheless, an active image of the breast tumor microenvironment is rapidly painted by the disclosed model, whereby stromal and immune cellular components react to an ever-changing environment driven by active malignancy.

### Prostate Adenocarcinoma.

Turning to prostate cancer (see FIG. 4C), the disclosed model (described above in conjunction with FIG. 1D) robustly distinguishes the tumor subtype, PRAD (Prostate Adenocarcinoma), and localizes malignant cell signatures within the invasive carcinoma region denoted in FIG. 4C—left, with nonmalignant luminal epithelial/basal cells in the lower-left region designated as Normal Gland. Fibromuscular zones outlined in green show distributions of myofibroblasts and smooth muscle cells. This sample contained a nerve fiber cross section, which the disclosed model detected as schwann cells, the myelinating cells of the peripheral nervous system [66]. PRAD is widely considered to be an immunologically “cold” tumor, compared to immunologically “hot” cancers such as melanoma [67,68]. Supporting this, the disclosed model did not detect meaningful presence of immune cells in the tested spatial section, and likewise PRAD ranks at the lowest end of absolute immune cell fractions among TCGA data deconvolved with UCD (see FIGS. 4M, 4N, and 4O).

Changes seen in prostate stromal tissue induced by carcinogenesis are mediated by cancer-activated fibroblasts (CAFs) adopting a myofibroblast-like phenotype [69]. Differentiating between myofibroblasts and conventional smooth muscle cells (SMCs) can be difficult as this phenotype is thought to reflect a continuum spanning conventional fibroblasts to mature prostatic SMCs [70]. Consequently, the disclosed model showed overlapping gene attributions used to differentiate these two highly-related cell types (see FIG. 4D).

Feature attributes reveal how the disclosed model learned to distinguish normal from cancerous prostate cells. Normal prostatic luminal epithelium was associated with KLK3 expression (see FIG. 4D). KLK3 encodes Prostate Serum Antigen (PSA), the most commonly used serum biomarker for prostate cancer despite suffering from low sensitivity due to its universal expression by both normal and malignant prostate cells. The disclosed model instead delineates prostate malignancy to KLK4, an intracellular kallikrein localizing to the nucleus providing markedly different functions from other KLK family genes [71]. Studies comparing KLK gene expression between prostate cancer and healthy controls have shown stronger statistical correlations between malignancy status and KLK4 compared with KLK3 [72].

### Colorectal Adenocarcinoma

Lastly, the disclosed model's deconvolution of colorectal adenocarcinoma was exampled (COAD, see FIG. 4E—right). Clear localization of COAD malignant cells is seen across presumptive tumor nodules shown in the unannotated H&E section in FIG. 4E—left. The stroma surrounding colorectal tumors has been shown to contain uniquely high proportions of infiltrating plasmablasts, a rapidly-dividing intermediate cell state representing activated B cells transitioned into mature, non-dividing plasma cells that function in an immunosuppressive role, which UCD readily detects in this sample [73]. Additional immune infiltrates identified by the disclosed model include macrophages and t cells sitting among fibroblast cells, highlighting the significant stromal immune responses commonly associated with pro-inflammatory tumor microenvironments.

### Example 11—Detecting Cell Type Compositional Changes in Pathological Bulk RNA-Seq Data

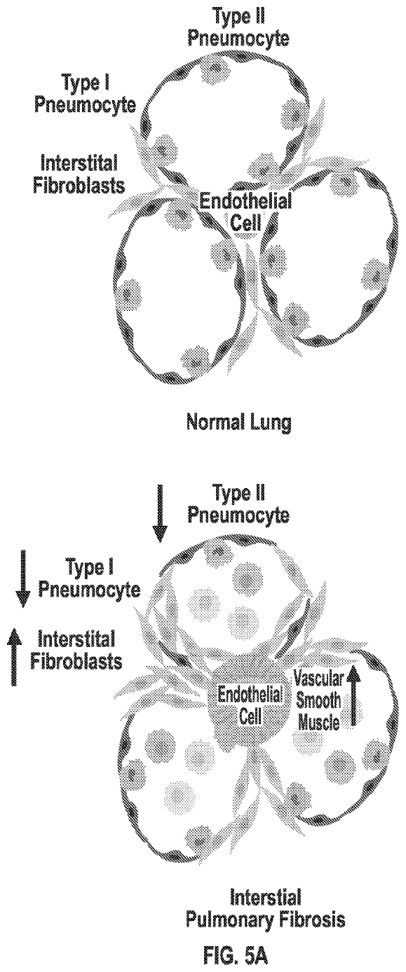

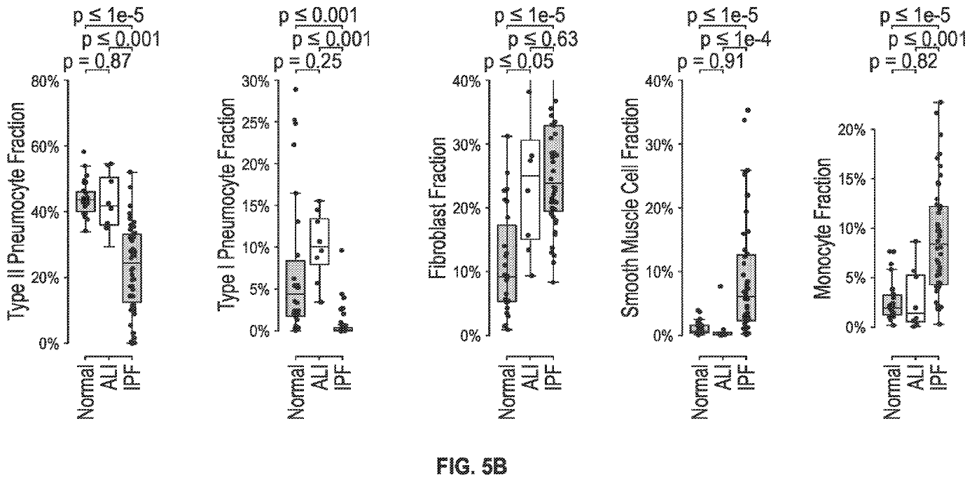

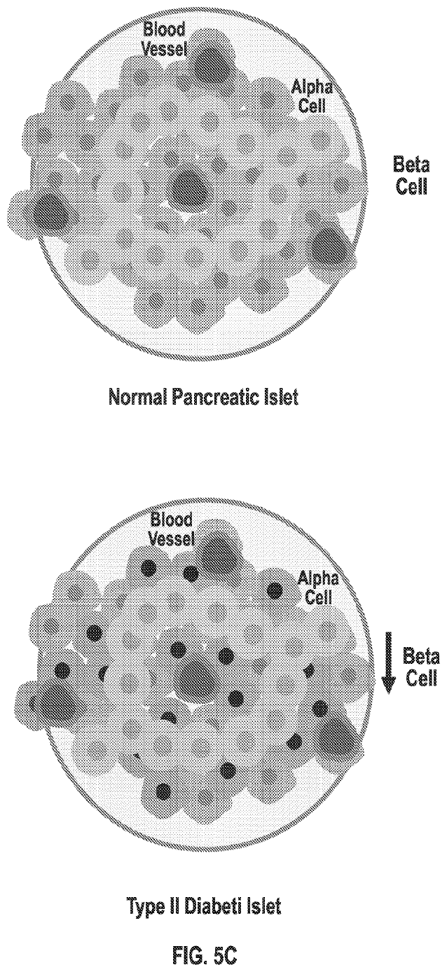

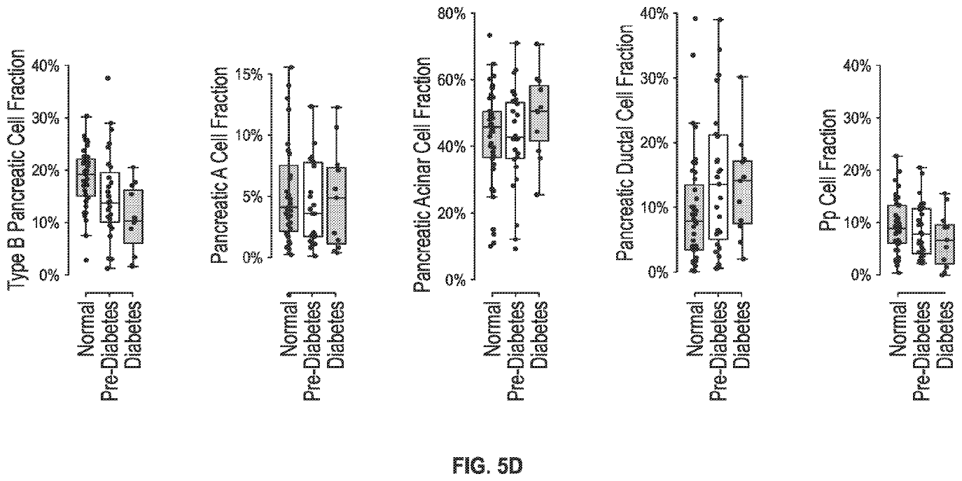

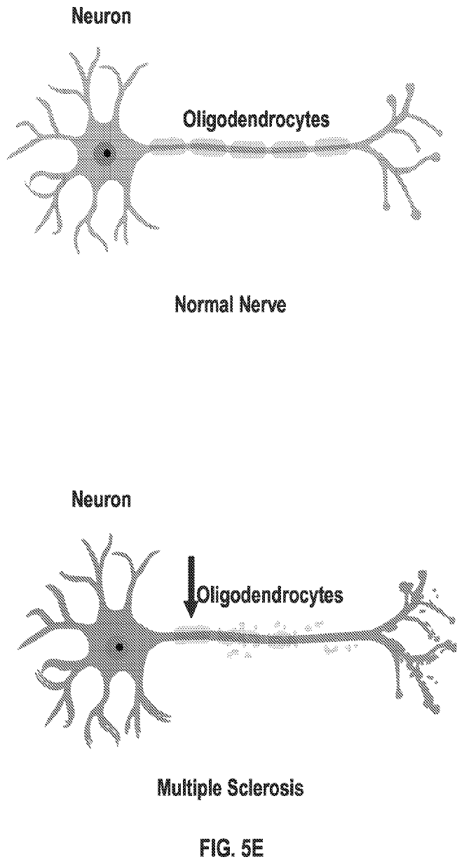

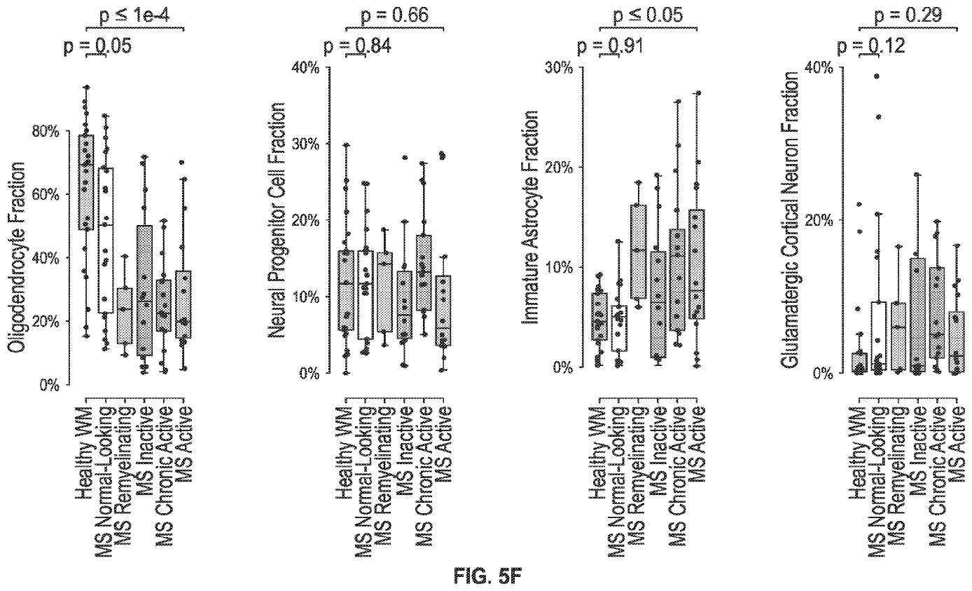

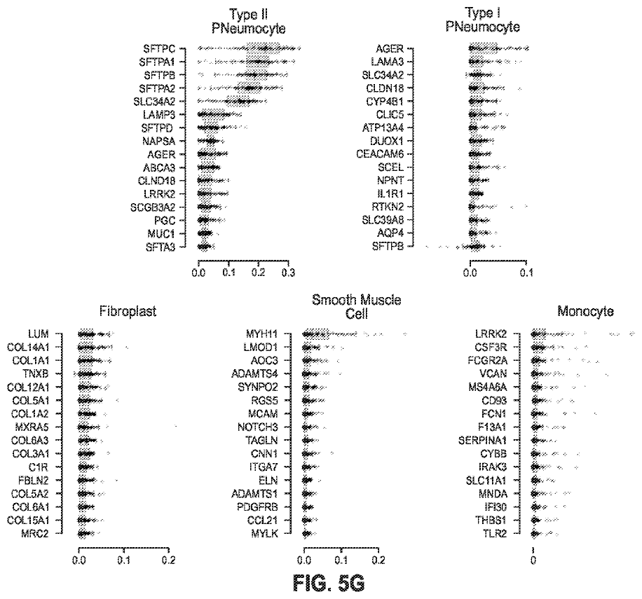

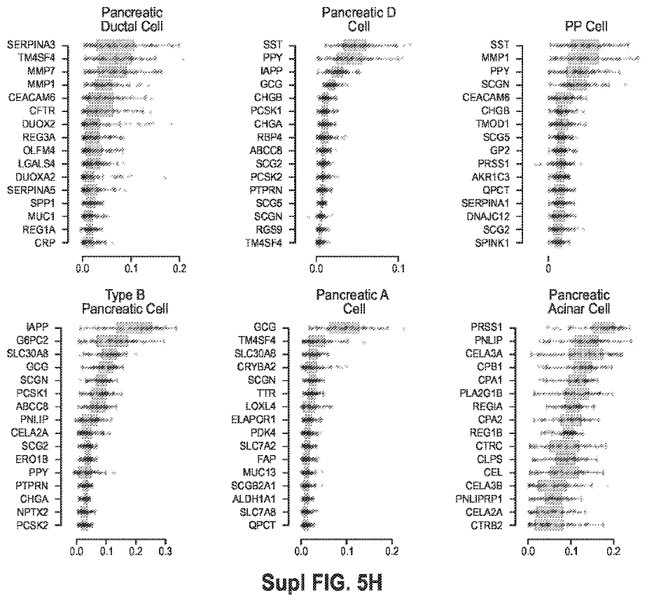

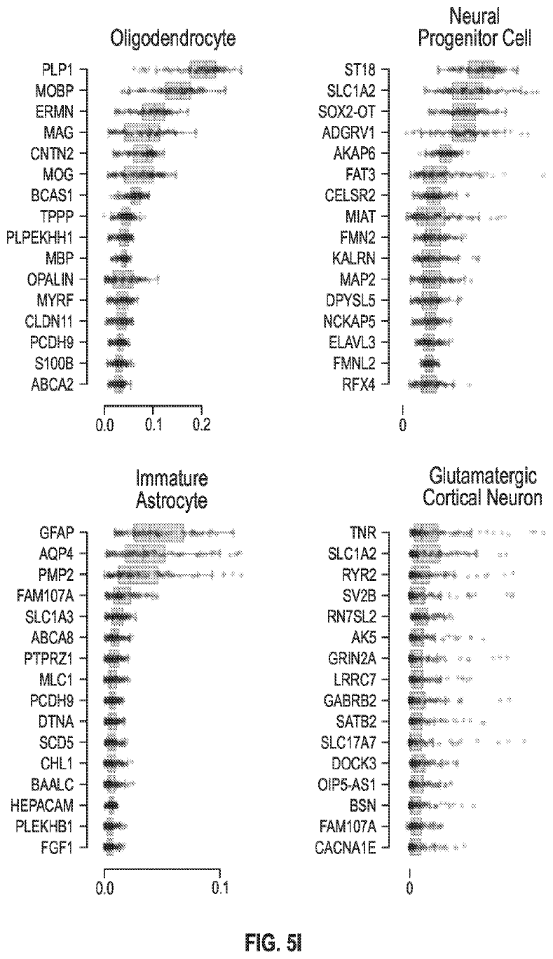

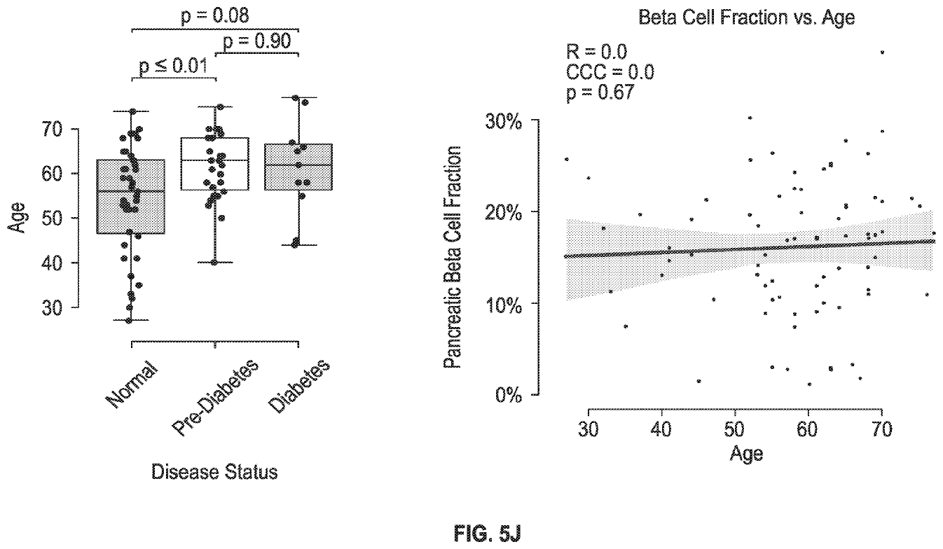

Given that scRNA-Seq and spatial transcriptomics remain cost-prohibitive for large-scale translational studies, bulk RNA-Seq data continues to dominate most clinical analyses. The disclosed model's (described above in conjunction with FIG. 1D) bulk-RNA-Seq ability to deconvolve bulk RNA-seq data to reveal pathologic changes in cellular fractions was studied. Feature attributes for each predicted cell type are shown in FIGS. 5G, 5H, and 5I, with detailed analysis of each feature's cellular relevance in Table 2 of Example 13.

Increased Fibromuscular Tissue Deposition in Idiopathic Pulmonary Fibrosis. Idiopathic pulmonary fibrosis (IPF) is a chronic lung disease characterized by the progressive inflammation, damage, and subsequent deposition of fibromuscular tissue into the lung interstitial space, and a corresponding destruction of the alveolar epithelium leading to a reduction in gas-exchange efficacy (see FIG. 5A) [74]. Acute lung injury (ALI), also known as acute respiratory distress syndrome (ARDS), is characterized by transient damage to the gas-exchange apparatus often induced by viral infection, and features significant fibrous tissue deposition as part of the tissue healing process [75].

Comparing Normal, ALI, and IPF tissues, significant reductions in fraction of Type II and Type I pneumocytes (ATII & ATI cells) in chronic IPF patient lungs (p<1E-5 ATII, p<0.001 ATI) were observed, with no difference seen between Normal and ALI (p=0.87 ATII, p=0.25 ATI). See FIG. 5B. This is consistent with the pathophysiologic destruction of alveolar epithelial cells in IPF. Fibroblast fractions were considerably higher for both ALI (p<0.05) and IPF (p<1E-5) patients compared to normal controls, consistent with the role that excessive fibroblast proliferation plays in IPF pathogenesis [76]. A significant increase in smooth muscle cell fractions (p<1E-5) was noted, defined by markers such as myosin heavy chain 11 (MYH11), occurring only in IPF patients. Pulmonary hypertension (PH) is a common secondary sequel to IPF, whereby excessive vascular smooth muscle deposition leads to elevated arterial pressure and potentially fatal cardiopulmonary consequences [77]. Interestingly, a distinct increase in monocyte fractions for IPF patients was seen (p<1E-5), a finding not seen in ALI. It has been previously reported that elevated monocyte count is associated with IPF progression and may play a role as a useful prognostic biomarker [78].

### Reduction of Pancreatic Beta Cells in Type II Diabetes

Type II diabetes mellitus (T2DM) is a disease characterized by the progressive increase in cellular insulin resistance, leading to a state of persistent hyperglycemia causing a chronic increase of insulin production [79]. The production stresses placed on pancreatic beta cells, responsible for insulin production in the body, eventually lead to apoptosis and selective reduction in beta cell fractions among pancreatic islets (see FIG. 5C) [80].

As T2DM progression exclusively impacts beta cells, differences in cell type fractions with respect to disease status were expected only among this cell type. Indeed, a clear, statistically significant decline in pancreatic beta cell fractions (p<0.01) was seen between normal and diabetes status (see FIG. 5D), with a strong downward trend among pre-diabetes patients correlating with disease progression. Beta cell fraction was not correlated to age in this cohort (p=0.67, see FIG. 5-J), although the rate of beta cell proliferation is known to decrease as age increases in the general population [81]. Examining other subpopulations of cell types present in pancreatic tissue (see FIGS. 5C and 5D), we no significant differences in Alpha, Delta, and PP (gamma) cells were seen, and similarly no differences in acinar and ductal cells forming the pancreatic glands.

### Reduced Oligodendrocyte Fractions in Chronic Multiple Sclerosis.

Multiple sclerosis (MS) is a chronic autoimmune disease affecting the central nervous system characterized by chronic inflammation induced by neural lymphocytic infiltration, which leads to progressive destruction of oligodendrocytes (the cells responsible for production of the myelin sheath) [82]. See FIG. 5E.

Significantly (p<1E-4) reduced oligodendrocyte fractions were seen when comparing control and active multiple sclerosis (MS) lesions. No significant changes to cortical neuron or neural progenitor cell fractions were noted; however, a weak (p<=0.05) increase in immature astrocytes between control and active MS were found. The proliferation of immature macroglial cells such as astrocytes has been associated with the neurotoxic effects of chronic inflammation induced by multiple sclerosis [83]. See FIG. 5F.

Overall, these results demonstrate that the disclosed model (described above in conjunction with FIG. 1D) was capable of faithfully recapitulating pathological changes in cell type fractions across a wide range of disease states. This robustness coupled with the validation offered by feature analysis makes the disclosed model a promising tool for the analysis of other pathologic bulk RNA-Seq datasets.

### Example 12—Rapid Cell Type Annotation & Disease Subtyping in Both Novel & Reference Non-Small Cell Lung Cancer scRNA-Seq Data

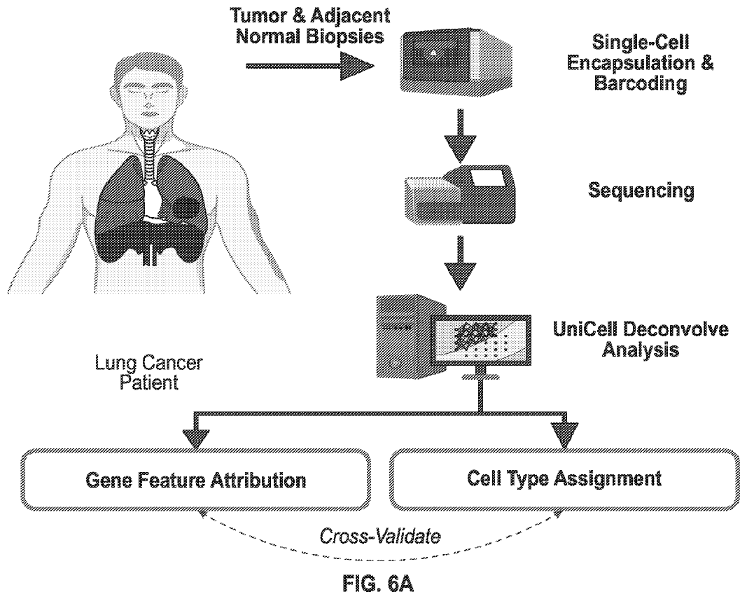

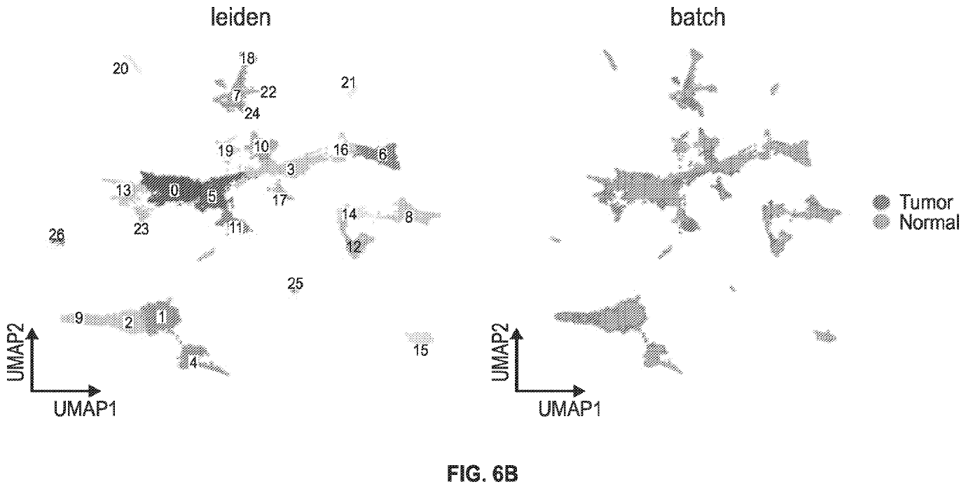

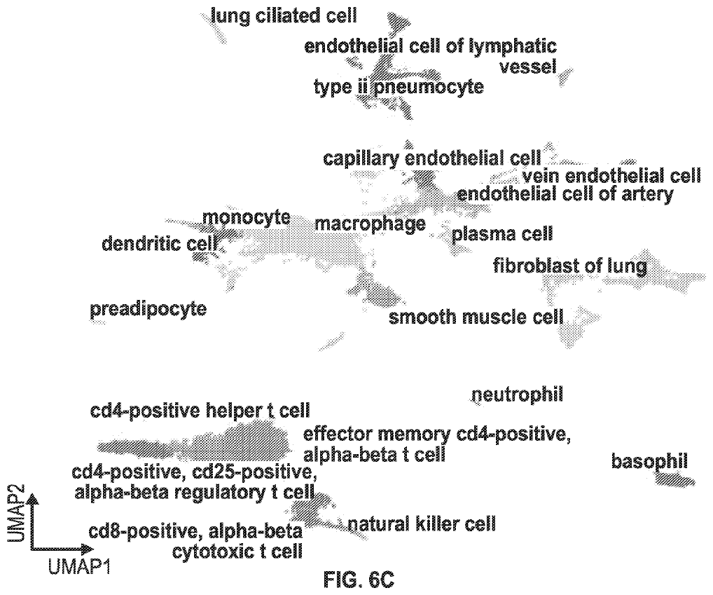

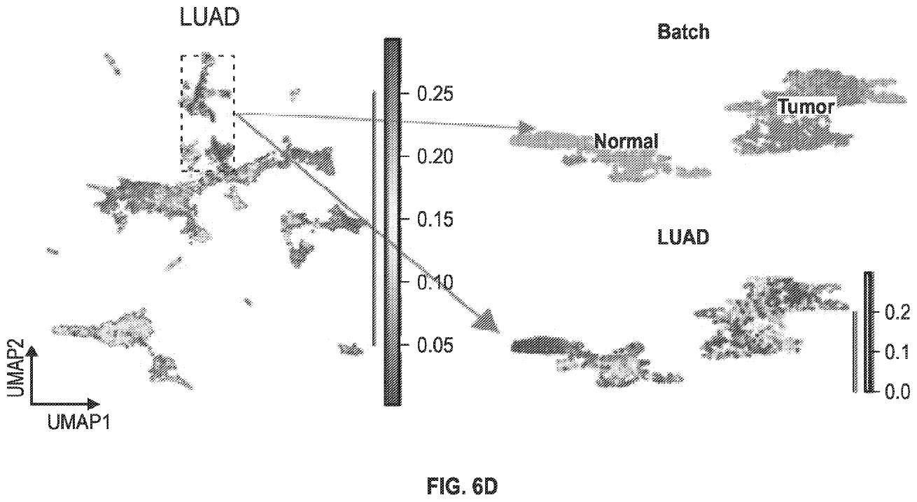

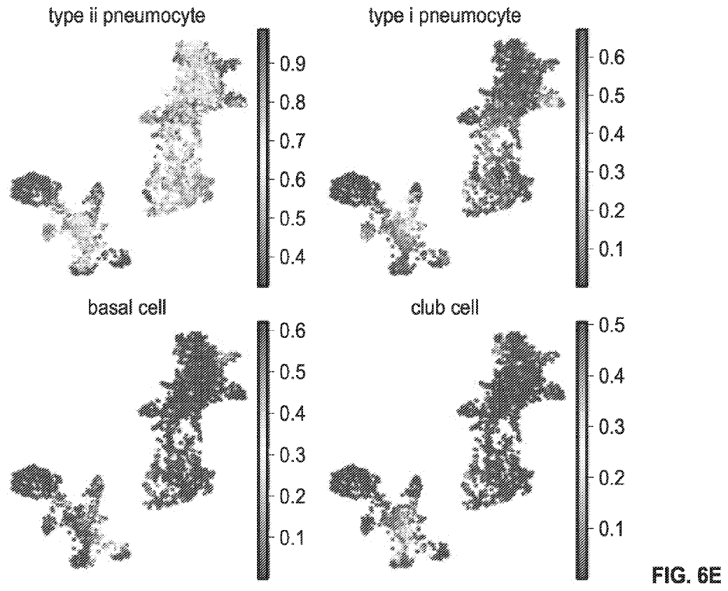

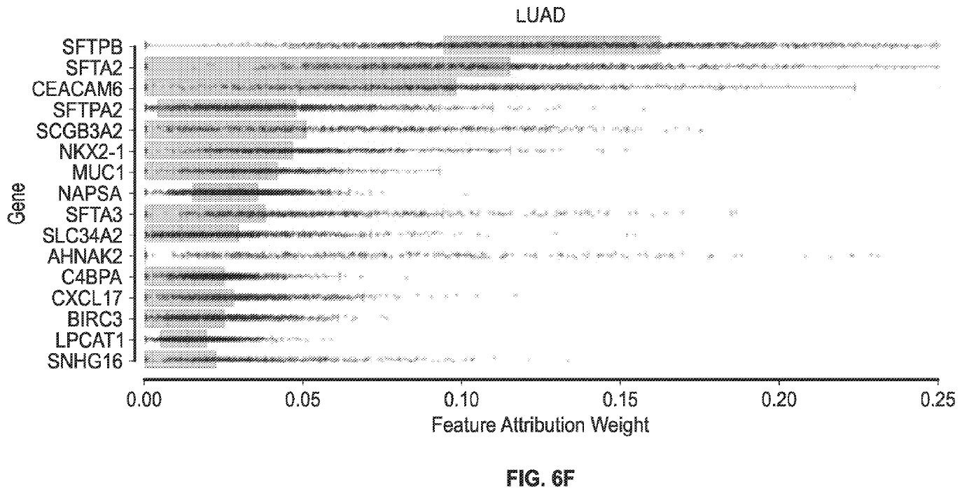

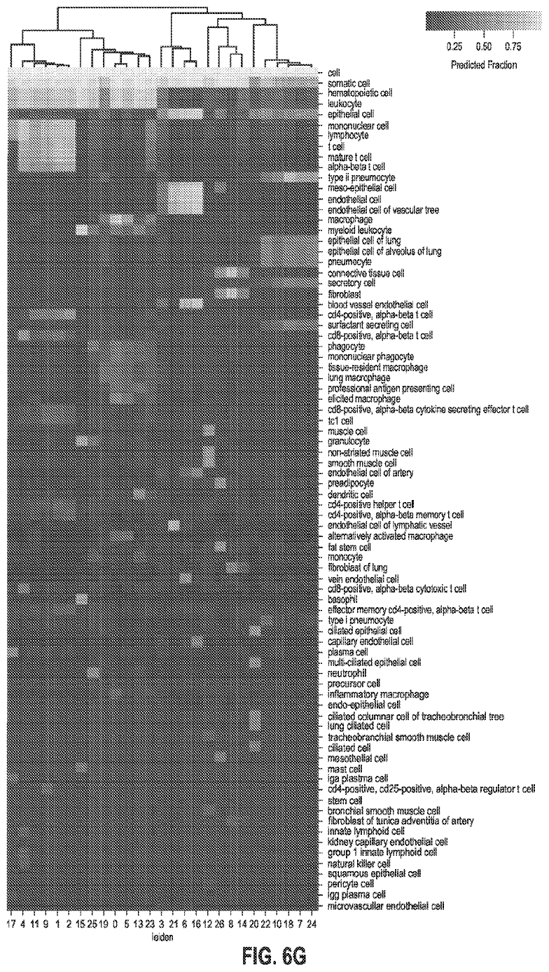

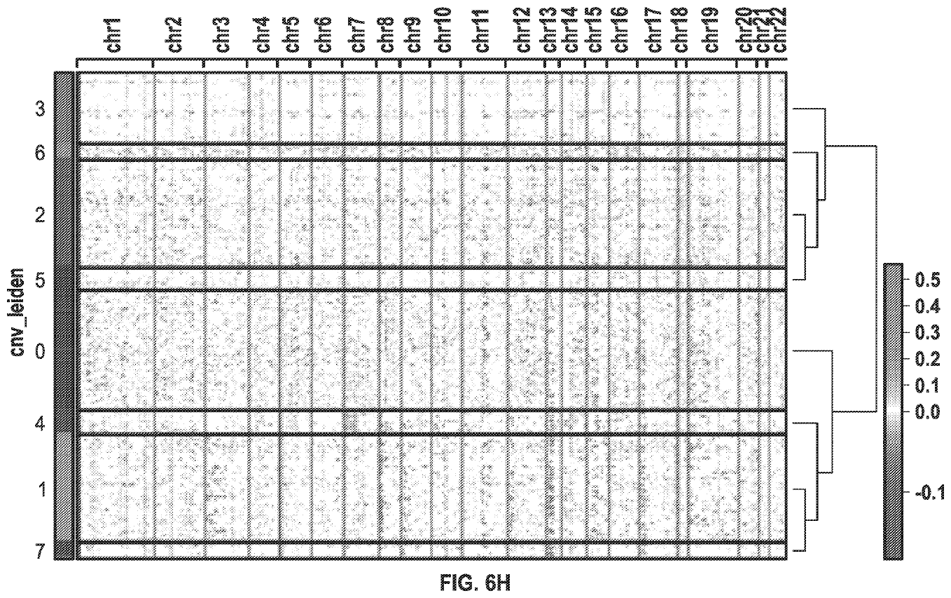

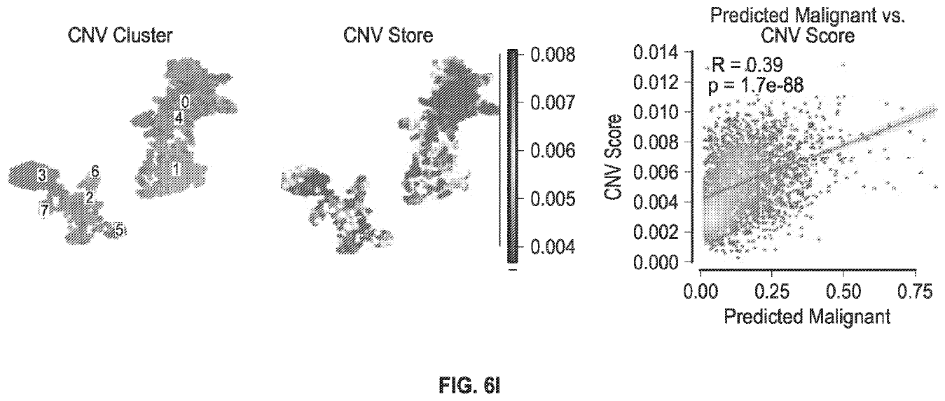

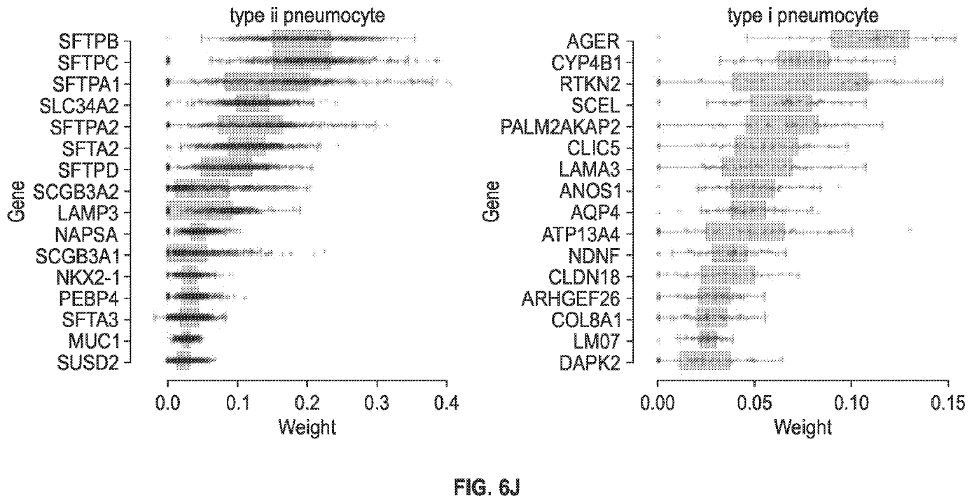

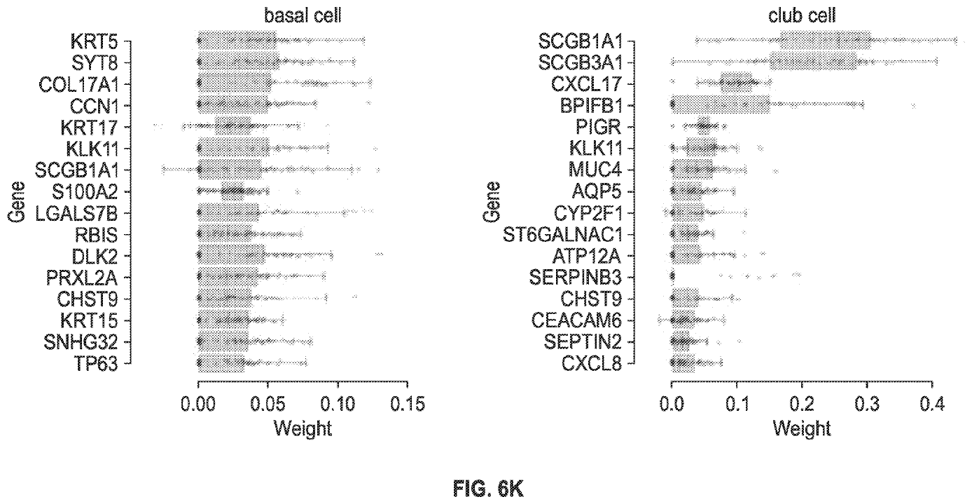

Given strong performance across spatial and bulk RNA-seq tissues, the disclosed model (described above in conjunction with FIG. 1D) was used to assist in basic cell type annotation of a non-small cell lung cancer scRNA-Seq dataset (see FIG. 6A), validating assigned cell types using feature attribution analysis (see FIG. 4N) followed by a literature analysis of identified markers (see Table 2 of Example 13).

Examining the annotated clusters (see FIGS. 6B and 6C) further, identification of malignant lung adenocarcinoma (LUAD) cell subpopulations among predicted epithelial cells were sought. This was found by the disclosed model (described above in conjunction with FIG. 1D) to likely be located within leiden clusters 18, 7, 22, 24, and 10 (see FIG. 6D—left). Because these clusters appeared intermixed with normal epithelial cells, this subset of cells was reclustered at higher resolution to reveal separations between malignant and nonmalignant cells (see FIG. 6D—right). Clear separation of cell clusters by biopsy status was observed, indicating most likely that tumor tissue contained a predominance of malignant cells. Indeed, the disclosed model predicted a higher probability of LUAD cells across the tumor biopsy derived cell clusters, with little to no malignant signal across cells derived from adjacent normal. To orthogonally validate malignancy predictions, copy-number variation (CNV) inference was performed, using a combination of smooth muscle, fibroblast, lung ciliated, and endothelial cells as reference controls, finding that the disclosed model's malignancy predictions overlapped estimated increased copy number variation (see FIGS. 6H and 6I). This relationship was quantified, finding considerably positive and significant correlation (spearman r=0.39, p=1.7E-88) between malignancy probability and average CNV score per cell (see FIG. 6I).

Some LUAD feature attributes (see FIG. 6F) were found to mirror surfactant genes related to type II pneumocytes, unsurprising as ATII cells are believed to be the cell of origin of LUAD [89]. A major malignancy-specific feature identified was carcinoembryonic antigen 6 (CEACAM6), known oncogenic gene overexpressed in numerous cancers including non-small cell lung (NSCLC), colon, and breast cancers [90]. Additional NSCLC-related genes identified include NKX2-1, a key transcription factor involved in early lung development and diagnostic marker for LUAD [91]. Non-malignant epithelial cells (see FIG. 6E) were clearly assigned to lung-related cell types with straightforward feature attributes corresponding to established cell type markers (see Table 2 of Example 13 for details). Overall, the disclosed model enabled the rapid and accurate annotation of a complex NSCLC patient case, with feature attribute analysis allowing for prospective validation of cell type assignment, in addition to delivering contextual information pertaining to the biological processes underpinning the data itself.

### Example 13—Cell Type Deconvolution Gene Feature Attribute Detailed Interpretations

As the disclosed models generate feature attributions that are specific to each sample being processed, attributed marker genes reflect contextualized biologic properties of cell types being predicted for that particular sample. For major cell types in each sample, the top sample-specific attributed marker genes are reported, and their biologic relevance to each study is reviewed as set forth in Table 2.

TABLE 2

| | | | Sample-Specific | | |
|---|---|---|---|---|---|
| | | | Attributed | | |
| FIG. | Tissue | Cell Type | Marker(s) | Relevance | Ref |
| 3 | Kidney | Proximal | SLC34A1 | The solute membrane transporter | [1] |
| | | Convoluted | | SLC34A1, a top predictor for PCT | |
| | | Tubule | | cells, is commonly overexpressed in | |
| | | Epithelial Cells | | the early cortical sections S1 & S2 of | |
| | | | | the PCT. | |
| | | Brush Cell | SLC6A18, | Both solute membrane transporters | [2, 3] |
| | | | SLC22A7 | 6A18 and 22A7 are known markers for | |
| | | | | the S3 PCT, which adopts a brush-cell | |
| | | | | like phenotype. | |
| | | Distal | SLC12A3 | Thiazide-sensitive sodium chloride | [4] |
| | | Convoluted | | cotransporter (NCC) encoded by | |
| | | Tubule | | SLC12A3, is expressed selectively in | |
| | | Epithelial Cell | | the distal convoluted tubule along the | |
| | | | | apical epithelial membrane. | |
| | | Thick | SLC12A1 | Solute carrier channel family 12 | [5] |
| | | Ascending | | member 1, a canonical marker for | |
| | | Limb of the | | TAL/LOH epithelial cells. | |
| | | Loop of Henle | | | |
| | | Collecting Duct | AQP2 | Water-reabsorption aquaporin channel | [6] |
| | | | | 2, a canonical marker for CD epithelial | |
| | | | | cells. | |
| | | Intercalated | ATP6V1G3 | An ATPase identified as a top | [7] |
| | | Cell | | differentially expressed IC cell gene | |
| | | | | compared with other kidney epithelial | |
| | | | | cells. | |
| | | Kidney | CFH | Complement Factor H plays a critical | [8] |
| | | Fibroblast Cells | | role in modulating the severity of | |
| | | | | innate immune activation following | |
| | | | | acute injury. | |
| | | Suppressor | MS4A7 | This membrane-bound complex | [9] |
| | | Macrophages | | protein is a known suppressor | |
| | | | | macrophage marker gene. | |
| | | | CCL8 | A chemokine thought to promote the | [10] |
| | | | | recruitment and polarization of M2- | |
| | | | | macrophages, supporting the | |
| | | | | establishment of auto/paracrine-like | |
| | | | | sustainment of chronic macrophage | |
| | | | | infiltration in late stage IRI and the | |
| | | | | establishment of chronic inflammation | |
| | | | | coinciding with fibrosis. | |
| | | | TREM2 | Believed to regulate macrophage | [11] |
| | | | | polarization in chronic kidney disease. | |
| | | T Cells | CCR7 | CCR7+ T cells play a role in mediating | [12] |
| | | | | kidney injury during transplant | |
| | | | | allograft rejection. | |
| 4 | Prostate | Prostate Cancer | NKX3-1 | An androgen-regulated homeodomain | [13] |
| | Single Cell | Single Cells | | gene localizing to prostate epithelium, | |
| | Database | (PRAD) | | which has shown to be positively | |
| | | | | expressed in the majority of primary | |
| | | | | prostate cancers. | |
| | | | PCA3 | Prostate cancer antigen 3 is a segment | [14] |
| | | | | of noncoding mRNA overexpressed in | |
| | | | | 95% or more prostate cancers. | |
| | | | FOLH1 | FOLH1 encodes prostate specific | [15] |
| | | | | membrane antigen (PSMA), a | |
| | | | | transmembrane protein with known | |
| | | | | carboxypeptidase activity that is | |
| | | | | commonly expressed in prostatic | |
| | | | | tissue, and overexpressed in prostate | |
| | | | | cancers. | |
| | Skin | Melanoma | MLANA | Codes for the melan-A protein are | [16] |
| | Cancer | Single Cells | | believed to play a functional role in | |
| | Single Cell | (SKCM) | | intracellular melanosome biogenesis, | |
| | Database | | | exclusively expressed in melanocytes, | |
| | | | | melanoma and retinal pigment | |
| | | | | epithelium. | |
| | | | TRYP1 | A tyrosinase-related protein found to | [17] |
| | | | | correlate with metastatic melanoma | |
| | | | | clinical outcomes. | |
| | | | MTRNR2L2 | A cancer-associated mitochondrial | [18] |
| | | | | related gene which codes for the anti- | |
| | | | | apoptotic peptide humanin. | |
| | Breast | Breast | SCGB2A2, | Secretoglobulins forming a protein | [19, 20] |
| | Adenocarcinoma | Adenocarcinoma | SCGB1D2 | complex commonly overexpressed in | |
| | Spatial | (BRCA) | | breast cancers. | |
| | Section | | PRLR | Codes for prolactin receptor, which is | [21] |
| | | | | overexpressed in a significant fraction | |
| | | | | of breast cancers and comprises one of | |
| | | | | the three major hormone receptors | |
| | | | | used for BC subtyping (ER, PR, and | |
| | | | | HER2). | |
| | | | ELAPOR1 | Endosome-lysosome autophagy | [22] |
| | | | | regulator 1 has been known to be | |
| | | | | overexpressed in several subtypes of | |
| | | | | cancer including breast, endometrial, | |
| | | | | and prostate cancers. | |
| | | | AZGP1 | An androgen-response secreted | [23] |
| | | | | glycoprotein which has been associated | |
| | | | | with several cancers including breast, | |
| | | | | prostate, and hepatocellular | |
| | | | | carcinomas. | |
| | | Fibroblast | LUM, COL1A1, | Well-established canonical fibroblast | [24] |
| | | | COL1A2, CO13A1, | markers representing various | |
| | | | FBLN2 | extracellular-matrix (ECM) genes | |
| | | | DPT | A secreted extracellular matrix | [25] |
| | | | | adhesion protein recently identified as | |
| | | | | a possible pan-tissue fibroblast marker. | |
| | | | C1R | Complement CR1 is a component of | [26] |
| | | | | the classical innate immune response | |
| | | | | pathway mediating local immuno- | |
| | | | | inflammatory responses. | |
| | | | COL12A1 | Implicated in pro-inflammatory | [27] |
| | | | | stromal desmoplasia and tumor | |
| | | | | progression | |
| | | Endothelial Cell | CDH5 | Endothelial-cell specific | [28] |
| | | | | transmembrane cadherin located along | |
| | | | | intercellular junctions. | |
| | | | APLN | Supports pro-angiogenic states among | [29] |
| | | | | endothelial cells, inducing migration | |
| | | | | and proliferation. | |
| | | Suppressor | MSR1 | Known to be overexpressed in breast | [30] |
| | | Macrophage | | cancer TAMs and has been associated | |
| | | | | with poor clinical outcomes. | |
| | | | CCL8 | C-C motif chemokine ligand 8 has | [31] |
| | | | | been shown to be secreted by TAMs to | |
| | | | | promote active tumorigenesis. | |
| | | IgG Plasma | IGHG1, IGHG2, | immunoglobulin heavy chains 1-4 are | [32] |
| | | Cell | IGHG3, IGHG4 | commonly overexpressed among IgG | |
| | | | | plasma cells. | |
| | | T Cell | TRAC | The t-cell receptor alpha constant gene | [33] |
| | | | | is a ubiquitous component of MHC | |
| | | | | complexes on all alpha-beta t cell | |
| | | | | subtypes. | |
| | Prostate | Prostate | KLK4 | An intracellular kallikrein localizing to | [34] |
| | Adenocarcinoma | Adenocarcinoma | | the nucleus and is believed to exert a | |
| | Spatial | (PRAD) | | pro-proliferative effect on prostate | |
| | Section | | | cancer cells via cell cycle signaling | |
| | | | | interactions. | |
| | | | OR51E2 | Ectopic olfactory G-protein coupled | [35, 36] |
| | | | | receptor is highly overexpressed in | |
| | | | | prostate cancers and may play a role in | |
| | | | | later-stage progression associated with | |
| | | | | neuroendocrine-like | |
| | | | | transdifferentiation | |
| | | Prostate | KLK2, KLK3 | KLK3 encodes Prostate Serum Antigen | [37] |
| | | Luminal | | (PSA), a secreted, chymotryptic-like | |
| | | Epithelial Cell | | enzyme involved in sperm cell | |
| | | | | maturation, which is cleaved from its | |
| | | | | zymogenic to active form by related | |
| | | | | secreted peptidase encoded by KLK2. | |
| | | | | Both genes are ubiquitously expressed | |
| | | | | in prostate luminal epithelial cells, both | |
| | | | | normal and cancerous. | |
| | | Prostatic Basal | TP63, KRT5 | Canonical basal cell marker genes. | [38, 39] |
| | | Cells | | TP63 regulates epithelial | |
| | | | | differentiation processes, while | |
| | | | | cytokeratin 5 forms intermediate | |
| | | | | filaments of the basal cell cytoskeleton. | |
| | | Schwann Cells | MPZ, CDH19, | Established schwann cell marker | [40] |
| | | | SOX10 | genes. Myelin-protein Z forms part of | |
| | | | | the myelin sheath that insulates nerve | |
| | | | | fibers. Cadherin 19 secures tight | |
| | | | | junctions between schwann cells, while | |
| | | | | SOX10 is a critical transcription factor | |
| | | | | essential to schwann cell identity. | |
| | | Myofibroblasts | CNN1, LMOD1 | Contractility promoting gene calponin- | [41] |
| | | | | 1 is known to be significantly | |
| | | | | upregulated in fibroblast populations | |
| | | | | that are treated with TGF-beta to | |
| | | | | induce myofibroblast-like | |
| | | | | differentiation, however it also plays a | |
| | | | | role in driving smooth muscle | |
| | | | | predictions | |
| | | Smooth Muscle | LMOD1, CNN1 | Leiomodin 1 is shown in recent studies | [42] |
| | | Cells | | to be expressed in only mature smooth | |
| | | | | muscle cells, although it does play a | |
| | | | | role, albeit smaller in myofibroblast | |
| | | | | predictions as well. | |
| | Colorectal | Colorectal | LGALS4, | Known COAD diagnostic marker | [43] |
| | Adenocarcinoma | Adenocarcinoma | CEACAM6 | genes. | |
| | Spatial | (COAD) | | | |
| | Section | Fibroblast | COL1A1, | Well-established canonical fibroblast | [24] |
| | | | COL1A2, LUM | markers representing various | |
| | | | | extracellular-matrix (ECM) genes. | |
| | | Plasmablasts | IGHG1-4 | immunoglobulin heavy chains 1-4 are | [44] |
| | | | | commonly overexpressed among | |
| | | | | plasma cells. | |
| | | | MZB1 | Supports a positive feedback loop with | [44, 45] |
| | | | | BLIMP1 to induce terminal | |
| | | | | differentiation of plasma cell | |
| | | | | phenotype. | |
| | | Macrophages | CCL8, CXCL10, | Involved in the attraction of t cells into | [46] |
| | | | CXCL9 | the tumor microenvironment via | |
| | | | | interaction with t-cell bound CXCR3 | |
| | | T Cell | CXCL10 | Secreted by t cells infiltrating tumor | [47] |
| | | | | micro movements as part of positive | |
| | | | | feedback loops maintaining tumor | |
| | | | | immune responses | |
| 5 | IPF, ALI | Type II | SFTPA1, SFTPC, | Encode surfactant proteins that | [48] |
| | Lung Bulk | Pneumocytes | SFTPB | function to coat the alveolar | |
| | RNA | (ATII) | | epithelium, supporting effective gas | |
| | Sample | | | exchange. Canonical ATII cell | |
| | | | | markers. | |
| | | Type I | AGER | Encodes advanced glycosylation end- | [49, 50] |
| | | Pneumocytes | | product specific receptor, | |
| | | (ATII) | | overexpressed in mature, differentiated | |
| | | | | ATI cells forming the majority of | |
| | | | | alveolar surface area. | |
| | | Fibroblast | COL1A1, LUM | Well-established canonical fibroblast | [24] |
| | | | | markers | |
| | | | MXRA5, | Matrix remodeling gene and collagen | [51, 52] |
| | | | COL14A1, | associated with lung fibrosis. | |
| | | Smooth Muscle | MYH11 | Myosin heavy chain 11 is a core | [53] |
| | | Cell | | component of smooth muscle cell | |
| | | | | contractile apparatus. | |
| | | Monocyte | LRRK2 | Expressed in various myeloid cell | [54] |
| | | | | populations and is associated with | |
| | | | | inflammatory disease processes. | |
| | Type 2 | Beta Cell | IAPP | Co-secreted with insulin and thought to | [55] |
| | Diabetes | | | be responsible for the accumulation of | |
| | Pancreatic | | | cytotoxic amyloid deposits | |
| | Bulk RNA | | | characteristic of type 2 diabetes | |
| | Sample | | | pathohistology, exacerbating cellular | |
| | | | | stress and eventually leading to beta | |
| | | | | cell death | |
| | | | G6PC2 | Selectively overexpressed in pancreatic | [56] |
| | | | | beta cells, serving to maintain high | |
| | | | | rates of intracellular glucose uptake | |
| | | Alpha Cell | GCG | Glucagon is selectively secreted by | [57] |
| | | | | pancreatic alpha cells to counteract | |
| | | | | effects of insulin from beta cells. | |
| | | Delta Cell | SST | Somatostatin is secreted by pancreatic | [58] |
| | | | | delta cells that is involved in the | |
| | | | | regulation of alpha nd beta cell | |
| | | | | activity. | |
| | | PP Cell | PPY, SST | It has been shown in mouse and rat | [59, 60] |
| | | | | studies that upwards of 60% of PP | |
| | | | | cells co-express SST in addition to | |
| | | | | canonical pancreatic polypeptide | |
| | | | | (PPY). | |
| | Multiple | Oligodendrocytes | PLP1 | Proteolipid protein 1 encodes a | [61] |
| | Sclerosis | | | transmembrane protein that forms the | |
| | Bulk RNA | | | primary component of myelin, | |
| | Samples | | | insulating neurons and improving | |
| | | | | action potential transduction. | |
| | | | MOBP | Myelin associated oligodendrocyte | [61] |
| | | | | basic protein is overexpressed in | |
| | | | | oligodendrocytes and forms an integral | |
| | | | | component of the myelin sheath. | |
| | | Immature | GFAP, AQP4 | traditional lineage-committed astrocyte | [62] |
| | | Astrocytes | | markers | |
| | | | FAM107A | Actin-binding protein that has | [63] |
| | | | | previously been reported to be | |
| | | | | overexpressed in astrocyte progenitor | |
| | | | | populations. | |
| 6 | Non-Small | CD4+ | FOXP3, CTLA4 | Characteristic markers of CD4 | [64] |
| | Cell Lung | Regulatory T | | regulatory t cells | |
| | Cancer | Cell | | | |
| | Biopsy | CD4+ Effector | IL7R | Required for the maintenance of | [65] |
| | | Memory T Cell | | memory t cell phenotypes | |
| | | CD8+ | GZMH, GZMB | Granzymes functioning to enable | [66] |
| | | Cytotoxic T | | cytotoxic behavior of t cells | |
| | | Cell | SEPTIN7 | Play a role in the related cytotoxic | [67]. |
| | | | | functions of immune cells. | |
| | | Natural Killer | KLRF1 | Well-known NK cell marker. | [68] |
| | | Cell | | | |
| | | Type I | AGER | Encodes advanced glycosylation end- | [49, 50] |
| | | Pneumocyte | | product specific receptor, | |
| | | | | overexpressed in mature, differentiated | |
| | | | | ATI cells forming the majority of | |
| | | | | alveolar surface area. | |
| | | Type II | SFTPC | Encode surfactant protein that | [48] |
| | | Pneumocyte | | functions to coat the alveolar | |
| | | | | epithelium. | |
| | | Basal Cells | KRT5 | cytokeratin 5 forms intermediate | [69] |
| | | | | filaments of the basal cell cytoskeleton. | |
| | | Club Cells | SCGB1A1, | Secretoglobulin proteins secreted by | [70] |
| | | | SCGB3A2 | lung airway epithelial cells, specific for | |
| | | | | club cell phenotypes. | |

References for Table 2:
[1]. Kusaba et al., 2014, “Differentiated kidney epithelial cells repair injured proximal tubule,” Proc Natl Acad Sci USA 111: 1527-1532.
[2]. Lindström et al., 2019, “Single-Cell Profiling Reveals Sex, Lineage, and Regional Diversity in the Mouse Kidney,” Dev Cell. 51: 399-413.e7.
[3]. Singer et al., 2009, “Orphan transporter SLC6A18 is renal neutral amino acid transporter B0AT3,” J Biol Chem. 2009; 284: 19953-19960.
[4]. Moes A D, van der Lubbe N, Zietse R, Loffing J, Hoorn E J. The sodium chloride cotransporter SLC12A3: new roles in sodium, potassium, and blood pressure regulation. Pflugers Arch. 2014; 466: 107-118.
[5]. Musso C G, Macías-Núñez J F. Dysfunction of the thick loop of Henle and senescence: from molecular biology to clinical geriatrics. Int Urol Nephrol. 2011; 43: 249-252.
[6]. Kwon T-H, Frøkiæ J, Nielsen S. Regulation of aquaporin-2 in the kidney: A molecular mechanism of body-water homeostasis. Kidney Res Clin Pract. 2013; 32: 96-102.
[7]. Saxena V, Fitch J, Ketz J, White P, Wetzel A, Chanley M A, et al. Whole Transcriptome Analysis of Renal Intercalated Cells Predicts Lipopolysaccharide Mediated Inhibition of Retinoid X Receptor alpha Function. Sci Rep. 2019; 9: 545.
[8]. Valoti E, Noris M, Perna A, Rurali E, Gherardi G, Breno M, et al. Impact of a Complement Factor H Gene Variant on Renal Dysfunction, Cardiovascular Events, and Response to ACE Inhibitor Therapy in Type 2 Diabetes. Front Genet. 2019; 10: 681.
[9]. Arlauckas S P, Garren S B, Garris C S, Kohler R H, Oh J, Pittet M J, et al. Arg1 expression defines immunosuppressive subsets of tumor-associated macrophages. Theranostics. 2018; 8: 5842-5854.
[10]. Sierra-Filardi E, Nieto C, Domínguez-Soto A, Barroso R, Sánchez-Mateos P, Puig-Kroger A, et al. CCL2 shapes macrophage polarization by GM-CSF and M-CSF: identification of CCL2/CCR2-dependent gene expression profile. J Immunol. 2014; 192: 3858-3867.
[11]. Cao Y, Qiancheng X, Cong F, Yuwei W. FP340 TREM-2 regulates macrophage polarization in chronic renal fibrosis. Nephrol Dial Transplant. 2019; 34: gfz106-FP340.
[12]. Kim K W, Kim B-M, Doh K C, Cho M-L, Yang C W, Chung B H. Clinical significance of CCR7+CD8+ T cells in kidney transplant recipients with allograft rejection. Sci Rep. 2018; 8: 8827.
[13]. Gurel B, Ali T Z, Montgomery E A, Begum S, Hicks J, Goggins M, et al. NKX3.1 as a marker of prostatic origin in metastatic tumors. Am J Surg Pathol. 2010; 34: 1097-1105.
[14]. Marks L S, Bostwick D G. Prostate Cancer Specificity of PCA3 Gene Testing: Examples from Clinical Practice. Rev Urol. 2008; 10: 175-181.
[15]. Chang S S. Overview of prostate-specific membrane antigen. Rev Urol. 2004; 6 Suppl 10: S13-8.
[16]. Du J, Miller A J, Widlund H R, Horstmann M A, Ramaswamy S, Fisher D E. MLANA/MART1 and SILV/PMEL17/GP100 are transcriptionally regulated by MITF in melanocytes and melanoma. Am J Pathol. 2003; 163: 333-343.
[17]. Journe F, Id Boufker H, Van Kempen L, Galibert M-D, Wiedig M, Salès F, et al. TYRP1 mRNA expression in melanoma metastases correlates with clinical outcome. Br J Cancer. 2011; 105: 1726-1732.
[18]. Bodzioch M, Lapicka-Bodzioch K, Zapala B, Kamysz W, Kiec-Wilk B, Dembinska-Kiec A. Evidence for potential functionality of nuclearly-encoded humanin isoforms. Genomics. 2009; 94: 247-256.
[19]. Zafrakas M, Petschke B, Donner A, Fritzsche F, Kristiansen G, Knüchel R, et al. Expression analysis of mammaglobin A (SCGB2A2) and lipophilin B (SCGB1D2) in more than 300 human tumors and matching normal tissues reveals their co-expression in gynecologic malignancies. BMC Cancer. 2006; 6: 88.
[20]. Talaat I M, Hachim M Y, Hachim I Y, Ibrahim R A E-R, Ahmed M A E R, Tayel H Y. Bone marrow mammaglobin-1 (SCGB2A2) immunohistochemistry expression as a breast cancer specific marker for early detection of bone marrow micrometastases. Sci Rep. 2020; 10: 13061.
[21]. Sleightholm R, Neilsen B K, Elkhatib S, Flores L, Dukkipati S, Zhao R, et al. Percentage of Hormone Receptor Positivity in Breast Cancer Provides Prognostic Value: A Single-Institute Study. J Clin Med Res. 2021; 13: 9-19.
[22]. Pontén F, Jirström K, Uhlen M. The Human Protein Atlas--a tool for pathology. J Pathol. 2008; 216: 387-393.
[23]. Tian H, Ge C, Zhao F, Zhu M, Zhang L, Huo Q, et al. Downregulation of AZGP1 by Ikaros and histone deacetylase promotes tumor progression through the PTEN/Akt and CD44s pathways in hepatocellular carcinoma. Carcinogenesis. 2017; 38: 207-217.
[24]. Muhl L, Genové G, Leptidis S, Liu J, He L, Mocci G, et al. Single-cell analysis uncovers fibroblast heterogeneity and criteria for fibroblast and mural cell identification and discrimination. Nat Commun. 2020; 11: 3953.
[25]. Zeltz C, Navab R, Heljasvaara R, Kusche-Gullberg M, Lu N, Tsao M-S, et al. Integrin α11β1 in tumor fibrosis: more than just another cancer-associated fibroblast biomarker? J Cell Commun Signal. 2022. doi: 10.1007/s12079-022-00673-3
[26]. Afshar-Kharghan V. The role of the complement system in cancer. J Clin Invest. 2017; 127: 780-789.
[27]. Jiang X, Wu M, Xu X, Zhang L, Huang Y, Xu Z, et al. COL12A1, a novel potential prognostic factor and therapeutic target in gastric cancer. Mol Med Rep. 2019; 20: 3103-3112.
[28]. Breviario F, Caveda L, Corada M, Martin-Padura I, Navarro P, Golay J, et al. Functional properties of human vascular endothelial cadherin (7B4/cadherin-5), an endothelium-specific cadherin. Arterioscler Thromb Vasc Biol. 1995; 15: 1229-1239.
[29]. Helker C S, Eberlein J, Wilhelm K, Sugino T, Malchow J, Schuermann A, et al. Apelin signaling drives vascular endothelial cells toward a pro-angiogenic state. Elife. 2020; 9. doi: 10.7554/eLife.55589
[30]. He Y, Zhou S, Deng F, Zhao S, Chen W, Wang D, et al. Clinical and transcriptional signatures of human CD204 reveal an applicable marker for the protumor phenotype of tumor-associated macrophages in breast cancer. Aging. 2019; 11: 10883-10901.
[31]. Zhang X, Chen L, Dang W-Q, Cao M-F, Xiao J-F, Lv S-Q, et al. CCL8 secreted by tumor-associated macrophages promotes invasion and stemness of glioblastoma cells via ERK1/2 signaling. Lab Invest. 2020; 100: 619-629.
[32]. Chen J, Tan Y, Sun F, Hou L, Zhang C, Ge T, et al. Single-cell transcriptome and antigen-immunoglobin analysis reveals the diversity of B cells in non-small cell lung cancer. Genome Biol. 2020; 21: 152.
[33]. TRAC T cell receptor alpha constant [Homo sapiens (human)] - Gene - NCBI. [cited 21 Feb. 2022]. Available: https://www.ncbi.nlm.nih.gov/gene/28755
[34]. Klokk T I, Kilander A, Xi Z, Waehre H, Risberg B, Danielsen H E, et al. Kallikrein 4 is a proliferative factor that is overexpressed in prostate cancer. Cancer Res. 2007; 67: 5221-5230.
[35]. Pronin A, Slepak V. Ectopically expressed olfactory receptors OR51E1 and OR51E2 suppress proliferation and promote cell death in a prostate cancer cell line. J Biol Chem. 2021; 296: 100475.
[36]. Abaffy T, Bain J R, Muehlbauer M J, Spasojevic I, Lodha S, Bruguera E, et al. A Testosterone Metabolite 19-Hydroxyandrostenedione Induces Neuroendocrine Trans-Differentiation of Prostate Cancer Cells via an Ectopic Olfactory Receptor. Front Oncol. 2018; 8: 162.
[37]. Adhyam M, Gupta A K. A Review on the Clinical Utility of PSA in Cancer Prostate. Indian J Surg Oncol. 2012; 3: 120-129.
[38]. Kurita T, Medina R T, Mills A A, Cunha G R. Role of p63 and basal cells in the prostate. Development. 2004; 131: 4955-4964.
[39]. Pignon J-C, Grisanzio C, Geng Y, Song J, Shivdasani R A, Signoretti S. p63-expressing cells are the stem cells of developing prostate, bladder, and colorectal epithelia. Proc Natl Acad Sci U S A. 2013; 110: 8105-8110.
[40]. Stratton J A, Kumar R, Sinha S, Shah P, Stykel M, Shapira Y, et al. Purification and Characterization of Schwann Cells from Adult Human Skin and Nerve. eNeuro. 2017; 4. doi: 10.1523/ENEURO.0307-16.2017
[41]. Scharenberg M A, Pippenger B E, Sack R, Zingg D, Ferralli J, Schenk S, et al. TGF-β-induced differentiation into myofibroblasts involves specific regulation of two MKL1 isoforms. J Cell Sci. 2014; 127: 1079-1091.
[42]. Nanda V, Miano J M. Leiomodin 1, a New Serum Response Factor-dependent Target Gene Expressed Preferentially in Differentiated Smooth Muscle Cells*. J Biol Chem. 2012; 287: 2459-2467.
[43]. Ferlizza E, Solmi R, Miglio R, Nardi E, Mattei G, Sgarzi M, et al. Colorectal cancer screening: Assessment of CEACAM6, LGALS4, TSPAN8 and COL1A2 as blood markers in faecal immunochemical test negative subjects. J Advert Res. 2020; 24: 99-107.
[44]. Andreani V, Ramamoorthy S, Pandey A, Lupar E, Nutt S L, Lämmermann T, et al. Cochaperone Mzb1 is a key effector of Blimp1 in plasma cell differentiation and β1-integrin function. Proc Natl Acad Sci U S A. 2018; 115: E9630-E9639.
[45]. Shaffer A L, Lin K I, Kuo T C, Yu X, Hurt E M, Rosenwald A, et al. Blimp-1 orchestrates plasma cell differentiation by extinguishing the mature B cell gene expression program. Immunity. 2002; 17: 51-62.
[46]. Tokunaga R, Zhang W, Naseem M, Puccini A, Berger M D, Soni S, et al. CXCL9, CXCL10, CXCL11/CXCR3 axis for immune activation - A target for novel cancer therapy. Cancer Treat Rev. 2018; 63: 40-47.
[47]. Peperzak V, Veraar E A M, Xiao Y, Babala N, Thiadens K, Brugmans M, et al. CD8+ T cells produce the chemokine CXCL10 in response to CD27/CD70 costimulation to promote generation of the CD8+ effector T cell pool. J Immunol. 2013; 191: 3025-3036.
[48]. Lee D F, Salguero F J, Grainger D, Francis R J, MacLellan-Gibson K, Chambers M A. Isolation and characterisation of alveolar type II pneumocytes from adult bovine lung. Sci Rep. 2018; 8: 11927.
[49]. Buckley S T, Ehrhardt C. The receptor for advanced glycation end products (RAGE) and the lung. J Biomed Biotechnol. 2010; 2010: 917108.
[50]. Garcia-de-Alba C, Pessina P, Kim C F. A new “age”r for lung research arrives: Genetic targeting of alveolar type 1 epithelial cells. American journal of respiratory cell and molecular biology. American Thoracic Society; 2018. pp. 661-662.
[51]. Yu D H, Ruan X-L, Huang J-Y, Liu X-P, Ma H-L, Chen C, et al. Analysis of the Interaction Network of Hub miRNAs-Hub Genes, Being Involved in Idiopathic Pulmonary Fibers and Its Emerging Role in Non-small Cell Lung Cancer. Front Genet. 2020; 11: 302.
[52]. Manon-Jensen T, Karsdal M A. Chapter 14 - Type XIV Collagen. In: Karsdal M A, editor. Biochemistry of Collagens, Laminins and Elastin. Academic Press; 2016. pp. 93-95.
[53]. Kwartler C S, Chen J, Thakur D, Li S, Baskin K, Wang S, et al. Overexpression of smooth muscle myosin heavy chain leads to activation of the unfolded protein response and autophagic turnover of thick filament-associated proteins in vascular smooth muscle cells. J Biol Chem. 2014; 289: 14075-14088.
[54]. Cabezudo D, Baekelandt V, Lobbestael E. Multiple-Hit Hypothesis in Parkinson's Disease: LRRK2 and Inflammation. Front Neurosci. 2020; 14: 376.
[55]. Kanatsuka A, Kou S, Makino H. IAPP/amylin and β-cell failure: implication of the risk factors of type 2 diabetes. Diabetol Int. 2018; 9: 143-157.
[56]. Bosma K J, Rahim M, Oeser J K, McGuinness O P, Young J D, O'Brien R M. G6PC2 confers protection against hypoglycemia upon ketogenic diet feeding and prolonged fasting. Mol Metab. 2020; 41: 101043.
[57]. Briant L, Salehi A, Vergari E, Zhang Q, Rorsman P. Glucagon secretion from pancreatic α-cells. Ups J Med Sci. 2016; 121: 113-119.
[58]. Hauge-Evans A C, King A J, Carmignac D, Richardson C C, Robinson I C A F, Low M J, et al. Somatostatin secreted by islet delta-cells fulfills multiple roles as a paracrine regulator of islet function. Diabetes. 2009; 58: 403-411.
[59]. Ludvigsen E, Olsson R, Stridsberg M, Janson E T, Sandler S. Expression and distribution of somatostatin receptor subtypes in the pancreatic islets of mice and rats. J Histochem Cytochem. 2004; 52: 391-400.
[60]. Perez-Frances M, van Gurp L, Abate M V, Cigliola V, Furuyama K, Bru-Tari E, et al. Pancreatic Ppy-expressing γ-cells display mixed phenotypic traits and the adaptive plasticity to engage insulin production. Nat Commun. 2021; 12: 4458.
[61]. Aston C, Jiang L, Sokolov B P. Transcriptional profiling reveals evidence for signaling and oligodendroglial abnormalities in the temporal cortex from patients with major depressive disorder. Mol Psychiatry. 2005; 10: 309-322.
[62]. Wallensten J, Nager A, Åsberg M, Borg K, Beser A, Wilczek A, et al. Leakage of astrocyte-derived extracellular vesicles in stress-induced exhaustion disorder: a cross-sectional study. Sci Rep. 2021; 11: 2009.
[63]. Sloan S A, Darmanis S, Huber N, Khan T A, Birey F, Caneda C, et al. Human Astrocyte Maturation Captured in 3D Cerebral Cortical Spheroids Derived from Pluripotent Stem Cells. Neuron. 2017; 95: 779-790.e6.
[64]. Barnes M J, Griseri T, Johnson A M F, Young W, Powrie F, Izcue A. CTLA-4 promotes Foxp3 induction and regulatory T cell accumulation in the intestinal lamina propria. Mucosal Immunol. 2013; 6: 324-334.
[65]. Belarif L, Mary C, Jacquemont L, Mai H L, Danger R, Hervouet J, et al. IL-7 receptor blockade blunts antigen-specific memory T cell responses and chronic inflammation in primates. Nat Commun. 2018; 9: 4483.
[66]. Patil V S, Madrigal A, Schmiedel B J, Clarke J, O'Rourke P, de Silva A D, et al. Precursors of human CD4+ cytotoxic T lymphocytes identified by single-cell transcriptome analysis. Sci Immunol. 2018; 3. doi: 10.1126/sciimmunol.aan8664
[67]. Phatarpekar P V, Overlee B L, Leehan A, Wilton K M, Ham H, Billadeau D D. The septin cytoskeleton regulates natural killer cell lytic granule release. J Cell Biol. 2020; 219. doi: 10.1083/jcb.202002145
[68]. Yang C, Siebert J R, Burns R, Gerbec Z J, Bonacci B, Rymaszewski A, et al. Heterogeneity of human bone marrow and blood natural killer cells defined by single-cell transcriptome. Nat Commun. 2019; 10: 3931.
[69]. Swatek A M, Lynch T J, Crooke A K, Anderson P J, Tyler S R, Brooks L, et al. Depletion of Airway Submucosal Glands and TP63+KRT5+ Basal Cells in Obliterative Bronchiolitis. Am J Respir Crit Care Med. 2018; 197: 1045-1057.
[70]. Naizhen X, Kido T, Yokoyama S, Linnoila R I, Kimura S. Spatiotemporal Expression of Three Secretoglobin Proteins, SCGB1A1, SCGB3A1, and SCGB3A2, in Mouse Airway Epithelia. J Histochem Cytochem. 2019; 67: 453-463.

### REFERENCES CITED AND ALTERNATIVE EMBODIMENTS

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

The present invention can be implemented as a computer program product that comprises a computer program mechanism embedded in a nontransitory computer readable storage medium. For instance, the computer program product could contain the program modules shown in FIG. 7 and/or described in FIGS. 8A, 8B, 8C, 8D, and/or 8E. These program modules can be stored on a CD-ROM, DVD, magnetic disk storage product, USB key, or any other non-transitory computer readable data or program storage product.

### REFERENCES

1. Casamassimi A, Federico A, Rienzo M, Esposito S, Ciccodicola A. Transcriptome Profiling in Human Diseases: New Advances and Perspectives. Int J Mol Sci. 2017; 18. doi: 10.3390/ijms18081652
2. Nomura S. Single-cell genomics to understand disease pathogenesis. J Hum Genet. 2021; 66:75-84.
3. Xia C, Fan J, Emanuel G, Hao J, Zhuang X. Spatial transcriptome profiling by MERFISH reveals subcellular RNA compartmentalization and cell cycle-dependent gene expression. Proc Natl Acad Sci USA. 2019; 116:19490-19499.
4. Goh J J L, Chou N, Seow W Y, Ha N, Cheng C P P, Chang Y-C, et al. Highly specific multiplexed RNA imaging in tissues with split-FISH. Nat Methods. 2020; 17:689-693.
5. Nguyen H Q, Chattoraj S, Castillo D, Nguyen S C, Nir G, Lioutas A, et al. 3D mapping and accelerated super-resolution imaging of the human genome using in situ sequencing. Nat Methods. 2020; 17:822-832.
6. Rodriques S G, Stickels R R, Goeva A, Martin C A, Murray E, Vanderburg C R, et al. Slide-seq: A scalable technology for measuring genome-wide expression at high spatial resolution. Science. 2019; 363:1463-1467.
7. Ståhl P L, Salmen F, Vickovic S, Lundmark A, Navarro J F, Magnusson J, et al. Visualization and analysis of gene expression in tissue sections by spatial transcriptomics. Science. 2016; 353:78-82.
8. Liu Y, Yang M, Deng Y, Su G, Enninful A, Guo C C, et al. High-Spatial-Resolution Multi-Omics Sequencing via Deterministic Barcoding in Tissue. Cell. 2020; 183:1665-1681.e18.
9. Chen A, Liao S, Cheng M, Ma K, Wu L, Lai Y, et al. Spatiotemporal transcriptomic atlas of mouse organogenesis using DNA nanoball-patterned arrays. Cell. 2022; 185:1777-1792.e21.
10. Zhong Y, Wan Y-W, Pang K, Chow L M L, Liu Z. Digital sorting of complex tissues for cell type-specific gene expression profiles. BMC Bioinformatics. 2013; 14:89.
11. Wang X, Park J, Susztak K, Zhang N R, Li M. Bulk tissue cell type deconvolution with multi-subject single-cell expression reference. Nat Commun. 2019; 10:380.
12. Newman A M, Steen C B, Liu C L, Gentles A J, Chaudhuri A A, Scherer F, et al. Determining cell type abundance and expression from bulk tissues with digital cytometry. Nat Biotechnol. 2019; 37:773-782.
13. Newman A M, Liu C L, Green M R, Gentles A J, Feng W, Xu Y, et al. Robust enumeration of cell subsets from tissue expression profiles. Nat Methods. 2015; 12:453-457.
14. Menden K, Marouf M, Oller S, Dalmia A, Magruder D S, Kloiber K, et al. Deep learning-based cell composition analysis from tissue expression profiles. Sci Adv. 2020; 6: eaba2619.
15. Gong T, Szustakowski J D. DeconRNASeq: a statistical framework for deconvolution of heterogeneous tissue samples based on mRNA-Seq data. Bioinformatics. 2013; 29:1083-1085.
16. Dong M, Thennavan A, Urrutia E, Li Y, Perou C M, Zou F, et al. SCDC: bulk gene expression deconvolution by multiple single-cell RNA sequencing references. Brief Bioinform. 2021; 22:416-427.
17. Kleshchevnikov V, Shmatko A, Dann E, Aivazidis A, King H W, Li T, et al. Cell2location maps fine-grained cell types in spatial transcriptomics. Nat Biotechnol. 2022. doi: 10.1038/s41587-021-01139-4.
18. Elosua-Bayes M, Nieto P, Mereu E, Gut I, Heyn H. SPOTlight: seeded NMF regression to deconvolute spatial transcriptomics spots with single-cell transcriptomes. Nucleic Acids Res. 2021. doi: 10.1093/nar/gkab043.
19. Andersson A, Bergenstrahle J, Asp M, Bergenstrahle L, Jurek A, Fernandez Navarro J, et al. Single-cell and spatial transcriptomics enables probabilistic inference of cell type topography. Commun Biol. 2020; 3:565.
20. Dong R, Yuan G-C. SpatialDWLS: accurate deconvolution of spatial transcriptomic data. Genome Biol. 2021; 22:145.
21. Song Q, Su J. DSTG: deconvoluting spatial transcriptomics data through graph-based artificial intelligence. Brief Bioinform. 2021; 22. doi: 10.1093/bib/bbaa414.
22. Miller B F, Huang F, Atta L, Sahoo A, Fan J. Reference-free cell-type deconvolution of multi-cellular pixel-resolution spatially resolved transcriptomics data. bioRxiv. 2021. p. 2021.06.15.448381. doi: 10.1101/2021.06.15.448381.
23. Cable D M, Murray E, Zou L S, Goeva A, Macosko E Z, Chen F, et al. Robust decomposition of cell type mixtures in spatial transcriptomics. Nat Biotechnol. 2021. doi: 10.1038/s41587-021-00830-w.
24. Avila Cobos F, Alquicira-Hernandez J, Powell J E, Mestdagh P, De Preter K. Benchmarking of cell type deconvolution pipelines for transcriptomics data. Nat Commun. 2020; 11:5650.
25. Vallania F, Tam A, Lofgren S, Schaffert S, Azad T D, Bongen E, et al. Leveraging heterogeneity across multiple datasets increases cell-mixture deconvolution accuracy and reduces biological and technical biases. Nat Commun. 2018; 9:4735.
26. De Veaux R D, Ungar L H. Multicollinearity: A tale of two nonparametric regressions. Selecting Models from Data. Springer New York; 1994. pp. 393-402.
27. Diehl A D, Meehan T F, Bradford Y M, Brush M H, Dahdul W M, Dougall D S, et al. The Cell Ontology 2016: enhanced content, modularization, and ontology interoperability. J Biomed Semantics. 2016; 7:44.
28. Numba: A high performance python compiler. [cited 9 Jan. 2022]. Available: https://numba.pydata.org/.
29. geo. Home-GEO-NCBI [cited 8 Jan. 2022]. Available:

https://www.ncbi.nlm.nih.gov/geo/.
30. EMBL-EBI. ArrayExpress. [cited 8 Jan. 2022]. Available: https://www.ebi.ac.uk/arrayexpress/.
31. UCSC Cell Browser. [cited 8 Jan. 2022]. Available: https://cells.ucsc.edu/?.
32. EBI Gene Expression Team-https://www.ebi.ac.uk/about/people/irene-papatheodorou. Single Cell Expression Atlas. [cited 8 Jan. 2022]. Available: https://www.ebi.ac.uk/gxa/sc/home.
33. Sun D, Wang J, Han Y, Dong X, Ge J, Zheng R, et al. TISCH: a comprehensive web resource enabling interactive single-cell transcriptome visualization of tumor microenvironment. Nucleic Acids Res. 2021; 49: D1420-D1430.
34. Home. [cited 8 Jan. 2022]. Available: https://www.humancellatlas.org/.
35. API docs. In: RAPIDS Docs [Internet]. [cited 9 Jan. 2022]. Available: https://docs.rapids.ai/api.
36. Tran H T N, Ang K S, Chevrier M, Zhang X, Lee N Y S, Goh M, et al. A benchmark of batch-effect correction methods for single-cell RNA sequencing data. Genome Biol. 2020; 21:12.
37. Korsunsky I, Millard N, Fan J, Slowikowski K, Zhang F, Wei K, et al. Fast, sensitive and accurate integration of single-cell data with Harmony. Nat Methods. 2019; 16:1289-1296.
38. Bolewski J, Papadopoulos S. Managing massive multi-dimensional array data with TileDB:—Invited demo paper. 2017 IEEE International Conference on Big Data (Big Data). IEEE; 2017. pp. 3175-3176.
39. Ding J, Regev A. Deep generative model embedding of single-cell RNA-Seq profiles on hyperspheres and hyperbolic spaces. Nat Commun. 2021; 12:2554.
40. De Cao N, Aziz W. The Power Spherical distribution. arXiv [stat. ML]. 2020. Available: http://arxiv.org/abs/2006.04437.
41. Franzén O, Gan L-M, Björkegren J L M. Panglao D B: a web server for exploration of mouse and human single-cell RNA sequencing data. Database. 2019; 2019. doi: 10.1093/database/baz046.
42. Zhang X, Lan Y, Xu J, Quan F, Zhao E, Deng C, et al. CellMarker: a manually curated resource of cell markers in human and mouse. Nucleic Acids Res. 2019; 47: D721-D728.
43. Lundberg S, Lee S-I. A Unified Approach to Interpreting Model Predictions. arXiv [cs.AI]. 2017. Available: http://arxiv.org/abs/1705.07874.
44. Ribeiro M T, Singh S, Guestrin C. “Why Should I Trust You?”: Explaining the Predictions of Any Classifier. arXiv [cs.LG]. 2016. Available: http://arxiv.org/abs/1602.04938.
45. Sundararajan M, Taly A, Yan Q. Axiomatic Attribution for Deep Networks. arXiv [cs.LG]. 2017. Available: http://arxiv.org/abs/1703.01365.
46. Dixon E E, Wu H, Muto Y, Wilson P C, Humphreys B D. Spatially Resolved Transcriptomic Analysis of Acute Kidney Injury in a Female Murine Model. J Am Soc Nephrol. 2022; 33:279-289.
47. Sivakumar P, Thompson J R, Ammar R, Porteous M, McCoubrey C, Cantu E 3rd, et al. RNA sequencing of transplant-stage idiopathic pulmonary fibrosis lung reveals unique pathway regulation. ERJ Open Res. 2019; 5. doi: 10.1183/23120541.00117-2019.
48. Elkjaer M L, Frisch T, Reynolds R, Kacprowski T, Burton M, Kruse T A, et al. Molecular signature of different lesion types in the brain white matter of patients with progressive multiple sclerosis. Acta Neuropathol Commun. 2019; 7:205.
49. Fadista J, Vikman P, Laakso E O, Mollet I G, Esguerra J L, Taneera J, et al. Global genomic and transcriptomic analysis of human pancreatic islets reveals novel genes influencing glucose metabolism. Proc Natl Acad Sci USA. 2014; 111:13924-13929.
50. Sherwani S I, Khan H A, Ekhzaimy A, Masood A, Sakharkar M K. Significance of HbA1c Test in Diagnosis and Prognosis of Diabetic Patients. Biomark Insights. 2016; 11:95-104.
51. Malek M, Nematbakhsh M. Renal ischemia/reperfusion injury; from pathophysiology to treatment. J Renal Inj Prev. 2015; 4:20-27.
52. Han S J, Lee H T. Mechanisms and therapeutic targets of ischemic acute kidney injury. Kidney Res Clin Pract. 2019; 38:427-440.
53. Saxena V, Gao H, Arregui S, Zollman A, Kamocka M M, Xuei X, et al. Kidney intercalated cells are phagocytic and acidify internalized uropathogenic Escherichia coli. Nat Commun. 2021; 12:2405.
54. Zhuo J L, Li X C. Proximal nephron. Compr Physiol. 2013; 3:1079-1123.
55. Kim K W, Kim B-M, Doh K C, Cho M-L, Yang C W, Chung B H. Clinical significance of CCR7+CD8+ T cells in kidney transplant recipients with allograft rejection. Sci Rep. 2018; 8:8827.
56. Huls M, van den Heuvel J J M W, Dijkman H B P M, Russel F G M, Masereeuw R. ABC transporter expression profiling after ischemic reperfusion injury in mouse kidney. Kidney Int. 2006; 69:2186-2193.
57. Bradner J E, Hnisz D, Young R A. Transcriptional Addiction in Cancer. Cell. 2017; 168:629-643.
58. Gurel B, Ali T Z, Montgomery E A, Begum S, Hicks J, Goggins M, et al. NKX3.1 as a marker of prostatic origin in metastatic tumors. Am J Surg Pathol. 2010; 34:1097-1105.
59. Du J, Miller A J, Widlund H R, Horstmann M A, Ramaswamy S, Fisher D E. MLANA/MARTI and SILV/PMEL17/GP100 are transcriptionally regulated by MITF in melanocytes and melanoma. Am J Pathol. 2003; 163:333-343.
60. Jiang X, Wu M, Xu X, Zhang L, Huang Y, Xu Z, et al. COL12A1, a novel potential prognostic factor and therapeutic target in gastric cancer. Mol Med Rep. 2019; 20:3103-3112.
61. Qiu S-Q, Waaijer S J H, Zwager M C, de Vries E G E, van der Vegt B, Schröder C P. Tumor-associated macrophages in breast cancer: Innocent bystander or important player? Cancer Treat Rev. 2018; 70:178-189.
62. Pidugu V K, Pidugu H B, Wu M-M, Liu C-J, Lee T-C. Emerging Functions of Human IFIT Proteins in Cancer. Front Mol Biosci. 2019; 6:148.
63. Zhang Y, Guan X-Y, Jiang P. Cytokine and Chemokine Signals of T-Cell Exclusion in Tumors. Front Immunol. 2020; 11:594609.
64. Ponten F, Jirstrom K, Uhlen M. The Human Protein Atlas—a tool for pathology. J Pathol. 2008; 216:387-393.
65. Galeano Niño J L, Pageon S V, Tay S S, Colakoglu F, Kempe D, Hywood J, et al. Cytotoxic T cells swarm by homotypic chemokine signalling. Elife. 2020; 9. doi: 10.7554/eLife.56554
66. Fallon M, Tadi P. Histology, Schwann Cells. 2019. Available: https://europepmc.org/article/nbk/nbk544316
67. Bou-Dargham M J, Sha L, Sang Q-XA, Zhang J. Immune landscape of human prostate cancer: immune evasion mechanisms and biomarkers for personalized immunotherapy. BMC Cancer. 2020; 20:572.
68. Li B, Severson E, Pignon J-C, Zhao H, Li T, Novak J, et al. Comprehensive analyses of tumor immunity: implications for cancer immunotherapy. Genome Biol. 2016; 17:174.
69. Hanley C J, Mellone M, Ford K, Thirdborough S M, Mellows T, Frampton S J, et al. Targeting the Myofibroblastic Cancer-Associated Fibroblast Phenotype Through Inhibition of NOX4. J Natl Cancer Inst. 2018; 110. doi: 10.1093/jnci/djx121
70. Kwon O-J, Zhang Y, Li Y, Wei X, Zhang L, Chen R, et al. Functional Heterogeneity of Mouse Prostate Stromal Cells Revealed by Single-Cell RNA-Seq. iScience. 2019; 13:328-338.
71. Klokk T I, Kilander A, Xi Z, Waehre H, Risberg B, Danielsen H E, et al. Kallikrein 4 is a proliferative factor that is overexpressed in prostate cancer. Cancer Res. 2007; 67:5221-5230.
72. Boyukozer F B, Tanoglu E G, Ozen M, Ittmann M, Aslan E S. Kallikrein gene family as biomarkers for recurrent prostate cancer. Croat Med J. 2020; 61:450-456.
73. Mao H, Pan F, Wu Z, Wang Z, Zhou Y, Zhang P, et al. Colorectal tumors are enriched with regulatory plasmablasts with capacity in suppressing T cell inflammation. Int Immunopharmacol. 2017; 49:95-101.
74. Sgalla G, Iovene B, Calvello M, Ori M, Varone F, Richeldi L. Idiopathic pulmonary fibrosis: pathogenesis and management. Respir Res. 2018; 19:32.
75. Marshall R, Bellingan G, Laurent G. The acute respiratory distress syndrome: fibrosis in the fast lane. Thorax. 1998. pp. 815-817.
76. Roberts M J, Broome R E, Kent T C, Charlton S J, Rosethorne E M. The inhibition of human lung fibroblast proliferation and differentiation by Gs-coupled receptors is not predicted by the magnitude of cAMP response. Respir Res. 2018; 19:56.
77. Ruffenach G, Hong J, Vaillancourt M, Medzikovic L, Eghbali M. Pulmonary hypertension secondary to pulmonary fibrosis: clinical data, histopathology and molecular insights. Respir Res. 2020; 21:303.
78. Kreuter M, Lee J S, Tzouvelekis A, Oldham J M, Molyneaux P L, Weycker D, et al. Monocyte Count as a Prognostic Biomarker in Patients with Idiopathic Pulmonary Fibrosis. Am J Respir Crit Care Med. 2021; 204:74-81.
79. Goyal R, Jialal I. Diabetes mellitus type 2. 2018. Available: https://europepmc.org/article/nbk/nbk513253
80. Cnop M, Welsh N, Jonas J-C, Jorns A, Lenzen S, Eizirik D L. Mechanisms of Pancreatic β-Cell Death in Type 1 and Type 2 Diabetes: Many Differences, Few Similarities. Diabetes. 2005; 54: S97-S107.
81. Helman A, Avrahami D, Klochendler A, Glaser B, Kaestner K H, Ben-Porath I, et al. Effects of ageing and senescence on pancreatic B-cell function. Diabetes Obes Metab. 2016; 18 Suppl 1:58-62.
82. Ghasemi N, Razavi S, Nikzad E. Multiple Sclerosis: Pathogenesis, Symptoms, Diagnoses and Cell-Based Therapy. Cell J. 2017; 19:1-10.
83. Correale J, Farez M F. The Role of Astrocytes in Multiple Sclerosis Progression. Front Neurol. 2015; 6:180.
84. Barnes M J, Griseri T, Johnson A M F, Young W, Powrie F, Izcue A. CTLA-4 promotes Foxp3 induction and regulatory T cell accumulation in the intestinal lamina propria. Mucosal Immunol. 2013; 6:324-334.
85. Patil V S, Madrigal A, Schmiedel B J, Clarke J, O'Rourke P, de Silva A D, et al. Precursors of human CD4+ cytotoxic T lymphocytes identified by single-cell transcriptome analysis. Sci Immunol. 2018; 3. doi: 10.1126/sciimmunol.aan8664
86. Phatarpekar P V, Overlee B L, Leehan A, Wilton K M, Ham H, Billadeau D D. The septin cytoskeleton regulates natural killer cell lytic granule release. J Cell Biol. 2020; 219. doi: 10.1083/jcb.202002145
87. Yang C, Siebert J R, Burns R, Gerbec Z J, Bonacci B, Rymaszewski A, et al. Heterogeneity of human bone marrow and blood natural killer cells defined by single-cell transcriptome. Nat Commun. 2019; 10:3931.
88. Belarif L, Mary C, Jacquemont L, Mai H L, Danger R, Hervouet J, et al. IL-7 receptor blockade blunts antigen-specific memory T cell responses and chronic inflammation in primates. Nat Commun. 2018; 9:4483.
89. Sainz de Aja J, Dost A F M, Kim C F. Alveolar progenitor cells and the origin of lung cancer. J Intern Med. 2021; 289:629-635.
90. Ru G-Q, Han Y, Wang W, Chen Y, Wang H-J, Xu W-J, et al. CEACAM6 is a prognostic biomarker and potential therapeutic target for gastric carcinoma. Oncotarget. 2017; 8:83673-83683.
91. Moisés J, Navarro A, Santasusagna S, Vinolas N, Molins L, Ramirez J, et al. NKX2-1 expression as a prognostic marker in early-stage non-small-cell lung cancer. BMC Pulm Med. 2017; 17:197.
92. Liu Y, Sun Y, Xue B, Zhang M, Yen G G, Tan K C. A Survey on Evolutionary Neural Architecture Search. IEEE Trans Neural Netw Learn Syst. 2021; PP. doi: 10.1109/TNNLS.2021.3100554

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

What is claimed:

1. A method of training a context-free model to determine a plurality of cell type fractions in a sample, the method comprising:
A) aggregating into a training set, for each respective data store in plurality of data stores,
for each respective cell in a respective population of cells represented in the respective data store,
a respective abundance dataset comprising a respective abundance value for each respective cellular constituent in a corresponding plurality of cellular constituents associated with the respective cell,
wherein
each data store in the plurality of data stores contributes a corresponding abundance dataset for each of a corresponding plurality of cells to the training set,
each corresponding plurality of cellular constituents in each respective cell is at least 50 cellular constituents,
the training set includes abundance data for twenty or more cell types, and
the training set includes abundance data for cells from ten or more tissue types;
B) forming a plurality of pseudobulk training mixtures from the training set, wherein each respective pseudobulk training mixture is formed by a first procedure comprising:
determining a number T on a random basis between a first lower threshold and a first upper threshold,
determining a number of unique cell types N between a second lower threshold and a second upper threshold,
determining a corresponding mixture fraction ratio Fᵢ on a random basis for each respective unique cell type i in the number of unique cells types N,
for each respective unique cell type i in the number of unique cell types N, selecting the abundance dataset of up to Fᵢ+T cells of the respective unique cell on a random basis from the training set thereby obtaining a respective plurality of cells represented by the respective pseudobulk training mixture, and
averaging the respective abundance value for each respective corresponding cellular constituent across each abundance dataset of the respective plurality of cells represented by the respective pseudobulk training mixture thereby forming an averaged abundance dataset for the respective pseudobulk training mixture comprising averaged abundance for a set of cellular constituents; and
C) training the context-free model, wherein the context-free model comprises a plurality of parameters, by performing for each respective pseudobulk training mixture in the plurality of pseudobulk training mixtures, a second procedure comprising:
inputting the averaged abundance dataset for the respective pseudobulk training mixture into the context-free model thereby obtaining a respective plurality of calculated cell type fractions, each calculated cell type fraction in the respective plurality of calculated cell type fractions for a different cell type in a plurality of cell types, and
adjusting a value of one or more parameters in the plurality of parameters based on a difference between (i) the respective plurality of calculated cell type fractions and (ii) the mixture fraction ratio Fᵢ for each respective unique cell type i in the number of unique cells types represented by the respective pseudobulk training mixture.

2. The method of claim 1, the method further comprising:
obtaining a test sample, in electronic form, wherein the test sample comprises a respective abundance value for each respective cellular constituent in a corresponding plurality of cellular constituents obtained from a bulk data assay; and
inputting the respective abundance value for each respective cellular constituent in the corresponding plurality of cellular constituents into the context-free model thereby obtaining a plurality of test calculated cell type fractions, each test calculated cell type fraction in the respective plurality of test calculated cell type fractions for a different cell type in the plurality of cell types.

3. The method of claim 1, the method further comprising:
obtaining a test sample, in electronic form, wherein the test sample comprises a respective abundance value for each respective cellular constituent in a corresponding plurality of cellular constituents for each respective test cell in a plurality of test cells obtained from a single cell or a single-nuclei assay; and
for each respective test cell in the plurality of test cells, inputting the respective abundance value for each respective cellular constituent in the corresponding plurality of cellular constituents into the context-free model thereby obtaining a respective plurality of test calculated cell type probabilities, each test calculated cell type probability in the respective plurality of test calculated cell type probabilities for a different cell type in the plurality of cell types.

4. The method of claim 3, the method further comprising:
averaging each respective plurality of test calculated cell type probabilities across the plurality of test cells to form a plurality of test calculated cell type fractions representative of the test sample, each respective test calculated cell type fraction in the plurality of test calculated cell type fractions corresponding to a different cell type in the plurality of cell types.

5. The method of any one of claims 1-4, wherein the plurality of cell types is between 50 cell types and 2000 cell types, or between 200 cell types and 1500 cell types, or between 500 cell types and 1000 cell types, greater than 100 cell types, greater than 200 cell types, or greater than 1000 cell types.

6. The method of any one of claims 1-5, wherein the respective plurality of cells represented by the respective pseudobulk training mixture includes cells from two or more data stores in the plurality of data stores.

7. The method of any one of claims 1-5, wherein the respective plurality of cells represented by the respective pseudobulk training mixture includes cells from three or more data stores in the plurality of data stores.

8. The method of any one of claims 1-5, wherein the respective plurality of cells represented by the respective pseudobulk training mixture includes cells from five or more data stores in the plurality of data stores.

9. The method of any one of claims 1-8, wherein the adjusting a value of one or more parameters in the plurality of parameters based on the difference is performed by backpropagation through all or a subset of the plurality of parameters of the context-free model.

10. The method of any one of claims 1-9, wherein the plurality of pseudobulk training mixtures comprises 100,000 pseudobulk training mixtures, 500,000 pseudobulk training mixtures, 1×10⁶ pseudobulk training mixtures, 5×10⁶ pseudobulk training mixtures, 1×10⁷ pseudobulk training mixtures, or 5×10⁷ pseudobulk training mixtures.

11. The method of any one of claims 1-10, the method further comprising repeating the training C) a plurality of times.

12. The method of any one of claims 1-10, the method further comprising repeating the training C) three or more times, four or more times, 10 or more times, between 15 and 100 times, or between 40 and 1000 times.

13. The method of any one of claims 1-12, wherein the context-free model is a multiple layer fully connected neural network.

14. The method of claim 13, wherein a final layer of the context-free model comprises a neuron for each cell type in the plurality of cell types with a softmax activation function yielding the respective plurality of calculated cell type fractions summing to one.

15. The method of any one of claims 1-14, wherein the plurality of data stores comprises 50 or more data stores, 100 or more data stores, or 1000 or more data stores.

16. The method of any one of claims 1-15, wherein the respective abundance dataset comprising a respective abundance value for each respective cellular constituent in the corresponding plurality of cellular constituents associated with the respective cell is single cell or single-nuclei RNA-seq data for a plurality of genes.

17. The method of any one of claims 1-15, wherein the respective abundance dataset comprising a respective abundance value for each respective cellular constituent in the corresponding plurality of cellular constituents associated with the respective cell is chromatin data.

18. The method of any one of claims 1-15, wherein the respective abundance dataset comprising a respective abundance value for each respective cellular constituent in the corresponding plurality of cellular constituents associated with the respective cell is protein expression data.

19. The method of any one of claims 1-18, wherein the plurality of trainable parameters comprises 1×10⁶ trainable parameters, 1×10⁷ trainable parameters, or 1×10⁸ trainable parameters.

20. The method of any one of claims 1-19, wherein the inputting the averaged abundance dataset for the respective pseudobulk training mixture into the context-free model during the training C) sets a first percentage of the set of cellular constituents to zero on a random basis.

21. The method of claim 20, wherein the first percentage is between 10 percent and 30 percent.

22. The method of any one of claims 1-21, wherein the set of cellular constituents consists of between 400 cellular constituents and 50,000 cellular constituents.

23. The method of any one of claims 1-22, wherein the context-free model comprises a plurality of fully connected layers, each respective fully connected layer in the plurality of fully connected layers comprising a corresponding plurality of neurons, and each respective neuron in the corresponding plurality of neurons analyzed by a corresponding activation function.

24. The method of claim 23, wherein the corresponding activation function is Tanh, sigmoid, softmax, Gaussian, Boltzmann-weighted averaging, absolute value, linear, rectified linear unit (ReLU), an exponential linear unit (ELU), a bounded rectified linear, soft rectified linear, parameterized rectified linear, average, max, min, sign, square, square root, multiquadric, inverse quadratic, inverse multiquadric, polyharmonic spline, swish, mish, Gaussian error linear unit (GeLU), and/or thin plate spline.

25. The method of claim 23 or 24, wherein the plurality of fully connected layers is between three and twenty fully connected layers.

26. The method of any one of claims 23-25, wherein there is at least one dropout layer between a first fully connected layer and a second fully connected layer in the plurality of fully connected layers that sets a second percentage of the neuron values of the first fully connected layer to zero on a random basis.

27. The method of claim 26, wherein the second percentage is between 5 percent and 15 percent of the of the neuron values of the first fully connected layer.

28. A computing system comprising one or more processors and a memory, the memory storing one or more programs for execution by the one or more processors, the one or more programs singularly or collectively comprising instructions for executing a method of training a context-free model to determine a plurality of cell type fractions in a sample, the method comprising:
A) aggregating, for each respective data store in plurality of data stores,
for each respective cell in a respective population of cells represented in the respective data store,
a respective abundance dataset comprising a respective abundance value for each respective cellular constituent in a corresponding plurality of cellular constituents associated with the respective cell, into a training set,
wherein
each data store in the plurality of data stores contributes an abundance dataset for each of a corresponding plurality of cells to the training set,
each corresponding plurality of cellular constituents in each respective cell is at least 50 cellular constituents,
the training set includes abundance data for twenty or more cell types, and
the training set includes abundance data for cells from ten or more tissue types;
B) forming a plurality of pseudobulk training mixtures from the training set, wherein each respective pseudobulk training mixture is formed by a first procedure comprising:
determining a number T on a random basis between a first lower threshold and a first upper threshold,
determining a number of unique cell types N between a second lower threshold and a second upper threshold,
determining a corresponding mixture fraction ratio Fᵢ on a random basis for each respective unique cell type i in the number of unique cells types N,
for each respective unique cell type i in the number of unique cell types N, selecting the abundance dataset of up to Fᵢ+T cells of the respective unique cell on a random basis from the training set thereby obtaining a respective plurality of cells represented by the respective pseudobulk training mixture, and
averaging the respective abundance value for each respective corresponding cellular constituent across each abundance dataset of the respective plurality of cells represented by the respective pseudobulk training mixture thereby forming an averaged abundance dataset for the respective pseudobulk training mixture comprising averaged abundance for a set of cellular constituents; and
C) training the context-free model, wherein the context-free model comprises a plurality of parameters, by performing for each respective pseudobulk training mixture in the plurality of pseudobulk training mixtures, a second procedure comprising:
inputting the averaged abundance dataset for the respective pseudobulk training mixture into the context-free model thereby obtaining a respective plurality of calculated cell type fractions, each calculated cell type fraction in the respective plurality of calculated cell type fractions for a different cell type in a plurality of cell types, and
adjusting a value of one or more parameters in the plurality of parameters based on a difference between (i) the respective plurality of calculated cell type fractions and (ii) the mixture fraction ratio Fᵢ for each respective unique cell type i in the number of unique cells types represented by the respective pseudobulk training mixture.

29. A non-transitory computer readable storage medium stored on a computing device, the computing device comprising one or more processors and a memory, the memory storing one or more programs for execution by the one or more processors, wherein the one or more programs singularly or collectively comprise instructions for executing a method of training a context-free model to determine a plurality of cell type fractions in a sample, the method comprising:
A) aggregating, for each respective data store in plurality of data stores,
for each respective cell in a respective population of cells represented in the respective data store,
a respective abundance dataset comprising a respective abundance value for each respective cellular constituent in a corresponding plurality of cellular constituents associated with the respective cell, into a training set,
wherein
each data store in the plurality of data stores contributes an abundance dataset for each of a corresponding plurality of cells to the training set,
each corresponding plurality of cellular constituents in each respective cell is at least 50 cellular constituents,
the training set includes abundance data for twenty or more cell types, and
the training set includes abundance data for cells from ten or more tissue types;
B) forming a plurality of pseudobulk training mixtures from the training set, wherein each respective pseudobulk training mixture is formed by a first procedure comprising:
determining a number T on a random basis between a first lower threshold and a first upper threshold,
determining a number of unique cell types N between a second lower threshold and a second upper threshold,
determining a corresponding mixture fraction ratio Fᵢ on a random basis for each respective unique cell type i in the number of unique cells types N,
for each respective unique cell type i in the number of unique cell types N, selecting the abundance dataset of up to Fᵢ+T cells of the respective unique cell on a random basis from the training set thereby obtaining a respective plurality of cells represented by the respective pseudobulk training mixture, and
averaging the respective abundance value for each respective corresponding cellular constituent across each abundance dataset of the respective plurality of cells represented by the respective pseudobulk training mixture thereby forming an averaged abundance dataset for the respective pseudobulk training mixture comprising averaged abundance for a set of cellular constituents; and
C) training the context-free model, wherein the context-free model comprises a plurality of parameters, by performing for each respective pseudobulk training mixture in the plurality of pseudobulk training mixtures, a second procedure comprising:
inputting the averaged abundance dataset for the respective pseudobulk training mixture into the context-free model thereby obtaining a respective plurality of calculated cell type fractions, each calculated cell type fraction in the respective plurality of calculated cell type fractions for a different cell type in a plurality of cell types, and
adjusting a value of one or more parameters in the plurality of parameters based on a difference between (i) the respective plurality of calculated cell type fractions and (ii) the mixture fraction ratio Fᵢ for each respective unique cell type i in the number of unique cells types represented by the respective pseudobulk training mixture.

30. A method of determining a plurality of cell type fractions for a plurality of cell types for a sample, the method comprising:
obtaining a test sample, in electronic form, wherein the test sample comprises a respective abundance value for each respective cellular constituent in a plurality of cellular constituents obtained from a bulk data assay; and
inputting the respective abundance value for each respective cellular constituent in the corresponding plurality of cellular constituents into a context-free model thereby obtaining a plurality of calculated cell type fractions, each calculated cell type fraction in the respective plurality of calculated cell type fractions for a different cell type in the plurality of cell types, wherein
the plurality of cell types comprises 300 or more different cell types,
the plurality of cellular constituents comprises 400 or more cellular constituents,
the context-free model comprises a plurality of fully connected layers, each respective fully connected layer in the plurality of fully connected layers comprising a corresponding plurality of neurons, and each respective neuron in the corresponding plurality of neurons analyzed by a corresponding activation function,
a final layer of the context-free model comprises a neuron for each cell type in the plurality of cell types with a softmax activation function yielding the respective plurality of calculated cell type fractions summing to one, and
the context-free model comprises 1×10⁶ trained parameters.

31. A computing system comprising one or more processors and a memory, the memory storing one or more programs for execution by the one or more processors, the one or more programs singularly or collectively comprising instructions for executing a method of determining a plurality of cell type fractions for a plurality of cell types for a sample, the method comprising:
obtaining a test sample, in electronic form, wherein the test sample comprises a respective abundance value for each respective cellular constituent in a plurality of cellular constituents obtained from a bulk data assay; and
inputting the respective abundance value for each respective cellular constituent in the corresponding plurality of cellular constituents into a context-free model thereby obtaining a plurality of calculated cell type fractions, each calculated cell type fraction in the respective plurality of calculated cell type fractions for a different cell type in the plurality of cell types, wherein
the plurality of cell types comprises 300 or more different cell types,
the plurality of cellular constituents comprises 400 or more cellular constituents,
the context-free model comprises a plurality of fully connected layers, each respective fully connected layer in the plurality of fully connected layers comprising a corresponding plurality of neurons, and each respective neuron in the corresponding plurality of neurons analyzed by a corresponding activation function,
a final layer of the context-free model comprises a neuron for each cell type in the plurality of cell types with a softmax activation function yielding the respective plurality of calculated cell type fractions summing to one, and
the context-free model comprises 1×10⁶ trained parameters.

32. A non-transitory computer readable storage medium stored on a computing device, the computing device comprising one or more processors and a memory, the memory storing one or more programs for execution by the one or more processors, wherein the one or more programs singularly or collectively comprise instructions for executing a method of determining a plurality of cell type fractions for a plurality of cell types for a sample, the method comprising:
obtaining a test sample, in electronic form, wherein the test sample comprises a respective abundance value for each respective cellular constituent in a plurality of cellular constituents obtained from a bulk data assay; and
inputting the respective abundance value for each respective cellular constituent in the corresponding plurality of cellular constituents into a context-free model thereby obtaining a plurality of calculated cell type fractions, each calculated cell type fraction in the respective plurality of calculated cell type fractions for a different cell type in the plurality of cell types, wherein
the plurality of cell types comprises 300 or more different cell types,
the plurality of cellular constituents comprises 400 or more cellular constituents,
the context-free model comprises a plurality of fully connected layers, each respective fully connected layer in the plurality of fully connected layers comprising a corresponding plurality of neurons, and each respective neuron in the corresponding plurality of neurons analyzed by a corresponding activation function,
a final layer of the context-free model comprises a neuron for each cell type in the plurality of cell types with a softmax activation function yielding the respective plurality of calculated cell type fractions summing to one, and
the context-free model comprises 1×10⁶ trained parameters.

33. A method of determining cell type proportions for a plurality of cell types for a sample, the method comprising:
obtaining a test sample, in electronic form, wherein the test sample comprises a respective abundance value for each respective cellular constituent in a corresponding plurality of cellular constituents for each respective test cell in a plurality of test cells obtained from a single cell assay; and
for each respective test cell in the plurality of test cells, inputting the respective abundance value for each respective cellular constituent in the corresponding plurality of cellular constituents into the context-free model thereby obtaining a respective plurality of test calculated cell type proportion, each test calculated cell type proportion in the respective plurality of test calculated cell type proportion for a different cell type in the plurality of cell types, wherein
the plurality of cell types comprises 300 or more different cell types,
the plurality of cellular constituents comprises 400 or more cellular constituents,
the context-free model comprises a plurality of fully connected layers, each respective fully connected layer in the plurality of fully connected layers comprising a corresponding plurality of neurons, and each respective neuron in the corresponding plurality of neurons analyzed by a corresponding activation function,
a final layer of the context-free model comprises a neuron for each cell type in the plurality of cell types with a softmax activation function yielding the respective plurality of calculated cell type proportions summing to one, and
the context-free model comprises 1×10⁶ trained parameters.

34. A computing system comprising one or more processors and a memory, the memory storing one or more programs for execution by the one or more processors, the one or more programs singularly or collectively comprising instructions for executing a method of determining cell type proportions for a plurality of cell types for a sample, the method comprising:
obtaining a test sample, in electronic form, wherein the test sample comprises a respective abundance value for each respective cellular constituent in a corresponding plurality of cellular constituents for each respective test cell in a plurality of test cells obtained from a single cell assay; and
for each respective test cell in the plurality of test cells, inputting the respective abundance value for each respective cellular constituent in the corresponding plurality of cellular constituents into the context-free model thereby obtaining a respective plurality of test calculated cell type proportion, each test calculated cell type proportion in the respective plurality of test calculated cell type proportion for a different cell type in the plurality of cell types, wherein
the plurality of cell types comprises 300 or more different cell types,
the plurality of cellular constituents comprises 400 or more cellular constituents,
the context-free model comprises a plurality of fully connected layers, each respective fully connected layer in the plurality of fully connected layers comprising a corresponding plurality of neurons, and each respective neuron in the corresponding plurality of neurons analyzed by a corresponding activation function,
a final layer of the context-free model comprises a neuron for each cell type in the plurality of cell types with a softmax activation function yielding the respective plurality of calculated cell type proportions summing to one, and
the context-free model comprises 1×10⁶ trained parameters.

35. A non-transitory computer readable storage medium stored on a computing device, the computing device comprising one or more processors and a memory, the memory storing one or more programs for execution by the one or more processors, wherein the one or more programs singularly or collectively comprise instructions for executing a method of determining cell type proportions for a plurality of cell types for a sample, the method comprising, the method comprising:
obtaining a test sample, in electronic form, wherein the test sample comprises a respective abundance value for each respective cellular constituent in a corresponding plurality of cellular constituents for each respective test cell in a plurality of test cells obtained from a single cell assay; and
for each respective test cell in the plurality of test cells, inputting the respective abundance value for each respective cellular constituent in the corresponding plurality of cellular constituents into the context-free model thereby obtaining a respective plurality of test calculated cell type proportion, each test calculated cell type proportion in the respective plurality of test calculated cell type proportion for a different cell type in the plurality of cell types, wherein
the plurality of cell types comprises 300 or more different cell types,
the plurality of cellular constituents comprises 400 or more cellular constituents,
the context-free model comprises a plurality of fully connected layers, each respective fully connected layer in the plurality of fully connected layers comprising a corresponding plurality of neurons, and each respective neuron in the corresponding plurality of neurons analyzed by a corresponding activation function,
a final layer of the context-free model comprises a neuron for each cell type in the plurality of cell types with a softmax activation function yielding the respective plurality of calculated cell type proportions summing to one, and
the context-free model comprises 1×10⁶ trained parameters.

36. The method of claim 30 or 33, wherein the context-free model is a multiple layer fully connected neural network.

37. The method of claim 36, wherein a final layer of the context-free model comprises a neuron for each cell type in the plurality of cell types with a softmax activation function yielding the respective plurality of calculated cell type fractions summing to one.

38. The computing system of claim 31 or 34, wherein the context-free model is a multiple layer fully connected neural network.

39. The computing system of claim 38, wherein a final layer of the context-free model comprises a neuron for each cell type in the plurality of cell types with a softmax activation function yielding the respective plurality of calculated cell type fractions summing to one.

40. The non-transitory computer readable storage medium of claim 32 or 35, wherein the context-free model is a multiple layer fully connected neural network.

41. The non-transitory computer readable storage medium of claim 40, wherein a final layer of the context-free model comprises a neuron for each cell type in the plurality of cell types with a softmax activation function yielding the respective plurality of calculated cell type fractions summing to one.

42. The method of claim 30 or 33, wherein the plurality of trainable parameters comprises 1×10⁷ trained parameters or 1×10⁸ trained parameters.

43. The computing system of claim 31 or 34, wherein the plurality of trainable parameters comprises 1×10⁷ trained parameters or 1×10⁸ trained parameters.

44. The non-transitory computer readable storage medium of claim 32 or 35, wherein the plurality of trainable parameters comprises 1×10⁷ trained parameters or 1×10⁸ trained parameters.

45. The method of claim 30 or 33, wherein the context-free model comprises a plurality of fully connected layers, each respective fully connected layer in the plurality of fully connected layers comprising a corresponding plurality of neurons, and each respective neuron in the corresponding plurality of neurons analyzed by a corresponding activation function.

46. The method of claim 45, wherein the corresponding activation function is Tanh, sigmoid, softmax, Gaussian, Boltzmann-weighted averaging, absolute value, linear, rectified linear unit (ReLU), an exponential linear unit (ELU), a bounded rectified linear, soft rectified linear, parameterized rectified linear, average, max, min, sign, square, square root, multiquadric, inverse quadratic, inverse multiquadric, polyharmonic spline, swish, mish, Gaussian error linear unit (GeLU), and/or thin plate spline.

47. The method of claim 45 or 46, wherein the plurality of fully connected layers is between three and twenty fully connected layers.

48. The computing system of claim 31 or 34, wherein the context-free model comprises a plurality of fully connected layers, each respective fully connected layer in the plurality of fully connected layers comprising a corresponding plurality of neurons, and each respective neuron in the corresponding plurality of neurons analyzed by a corresponding activation function.

49. The computing system of claim 48, wherein the corresponding activation function is Tanh, sigmoid, softmax, Gaussian, Boltzmann-weighted averaging, absolute value, linear, rectified linear unit (ReLU), an exponential linear unit (ELU), a bounded rectified linear, soft rectified linear, parameterized rectified linear, average, max, min, sign, square, square root, multiquadric, inverse quadratic, inverse multiquadric, polyharmonic spline, swish, mish, Gaussian error linear unit (GeLU), and/or thin plate spline.

50. The computing system of claim 48 or 49, wherein the plurality of fully connected layers is between three and twenty fully connected layers.

51. The non-transitory computer readable storage medium of claim 32 or 35, wherein the context-free model comprises a plurality of fully connected layers, each respective fully connected layer in the plurality of fully connected layers comprising a corresponding plurality of neurons, and each respective neuron in the corresponding plurality of neurons analyzed by a corresponding activation function.

52. The non-transitory computer readable storage medium of claim 51, wherein the corresponding activation function is Tanh, sigmoid, softmax, Gaussian, Boltzmann-weighted averaging, absolute value, linear, rectified linear unit (ReLU), an exponential linear unit (ELU), a bounded rectified linear, soft rectified linear, parameterized rectified linear, average, max, min, sign, square, square root, multiquadric, inverse quadratic, inverse multiquadric, polyharmonic spline, swish, mish, Gaussian error linear unit (GeLU), and/or thin plate spline.

53. The non-transitory computer readable storage medium of claim 51 or 52, wherein the plurality of fully connected layers is between three and twenty fully connected layers.
